(19) United States
(12) Patent Application Publication
(10) Pub. No.: US 2012/0214818 A1
(43) Pub. Date: Aug. 23, 2012

(54) METHODS OF TREATING DIASTOLIC DYSFUNCTION AND RELATED CONDITIONS

(75) Inventor: Samuel Dudley, Chicago, IL (US)

(73) Assignee: THE BOARD OF TRUSTEES OF THE UNIVERSITY OF ILLINOIS, Urbana, IL (US)

(21) Appl. No.: 13/397,622

(22) Filed: Feb. 15, 2012

Related U.S. Application Data
(63) Continuation-in-part of application No. PCT/US10/48650, filed on Sep. 13, 2010.
(60) Provisional application No. 61/241,585, filed on Sep. 11, 2009.
(60) Provisional application No. 61/263,920, filed on Nov. 24, 2009.
(60) Provisional application No. 61/348,105, filed on May 25, 2010.
(60) Provisional application No. 61/443,378, filed on Feb. 16, 2011.

(51) Int. Cl. A61K 31/495; A61P 9/00

(52) U.S. Cl. 514/252.12

(57) ABSTRACT

The invention provides a method of treating diastolic dysfunction, e.g., diastolic dysfunction with preserved ejection fraction, in a subject. The method comprises administering to the subject in an amount effective to treat the diastolic dysfunction a cardiac metabolic modifier, as described herein. In some embodiments, the diastolic dysfunction is characterized by (i) a lack of increased late I_{Na} in cardiomyocytes, (ii) an increase in myofilament calcium sensitivity, or (iii) a combination thereof. In some embodiments, the subject does not suffer from a cardiac injury or a structural heart disease, as described herein. Further provided are a method of treating heart failure with preserved ejection fraction in a subject, a method of treating acute decompensated heart failure, a method of modulating myofilament calcium sensitivity in a subject, and a method of treating a condition associated with or caused by increased myofilament calcium sensitivity.

### CROSS-REFERENCE TO RELATED APPLICATIONS

This application claims priority to U.S. Provisional Application No. 61/443,378, filed on Feb. 16, 2011, and to International Patent Application No. PCT/US2010/048650, filed on Sep. 13, 2010, which claims the benefit of U.S. Provisional Patent Application No. 61/241,585, filed on Sep. 11, 2009, U.S. Provisional Patent Application No. 61/263,920, filed on Nov. 24, 2009, and U.S. Provisional Patent Application No. 61/348,105, filed on May 25, 2010, each application of which is incorporated by reference in their entirety.

### GRANT FUNDING

This invention was made with government support under Grant Nos. R01 HL085558, R01 HL073753, P01 HL058000, T32 HL007692, R01 HL090851, ARRA supplement R01 HL090851-0251, RO1 HL022231, RO1HL062426, and RO1 HL064035, awarded by the National Institutes of Health, a Veterans Affairs MERIT grant. The government has certain rights in the invention.

### BACKGROUND

Diastolic dysfunction is characterized by prolonged relaxation of the myocardium and, if untreated, can lead to the clinical syndrome, heart failure with preserved ejection fraction (HFpEF). HFpEF is an increasingly prevalent health burden accounting for significant morbidity, mortality, and healthcare expenditures.¹⁻⁴ The underlying mechanisms in diastolic dysfunction are not clearly understood, limiting treatment options.⁵ Recent large clinical trials using the standard therapies for systolic heart failure have failed to demonstrate improvement, further emphasizing differences in the underlying pathophysiology of diastolic dysfunction.⁶⁻⁸

There are several potential mechanisms for diastolic dysfunction. One potential mechanism for diastolic dysfunction is increased diastolic Ca²⁺ resulting in a slowed ventricular relaxation and diastolic dysfunction. Ca²⁺ is removed from the cytosol during diastole by the sarcoplasmic reticular Ca²⁺-ATPase (SERCA) and the Na'/Ca²⁺ exchanger (NCX). The NCX couples Ca²⁺ extrusion to the transmembrane Na⁺ gradient.⁹ In the failing heart, a small number of the Na⁺ channels fail to inactivate creating a late Na⁺ current (I_{Na}).¹⁰⁻¹³ The late I_{Na} increases Na⁺ entry into the cell, reducing Ca²⁺ extrusion by NCX.¹⁴ Oxidative stress¹⁵ and myo filament Ca²⁺ sensitization represent other potential mechanisms for diastolic dysfunction, and, may represent other forms or sub-types of diastolic dysfunction.

Because there are several potential mechanisms for diastolic dysfunction, it is possible that sub-types of this medical condition exist, such that successful treatment of a patient exhibiting diastolic dysfunction may depend on the particular sub-type of diastolic dysfunction the patient presents.

While studies have demonstrated an improvement of a sub-type of diastolic dysfunction in which the late I_{Na} in failing hearts was increased, the relevance of this sub-type of diastolic dysfunction to the form of diastolic dysfunction presented by humans is questioned, since most of these studies were carried out in animal models in which the increased late I_{Na} or diastolic dysfunction was artificially induced, e.g., toxin-induced diastolic dysfuntion, ischemia-induced diastolic dysfunction.

To date, no studies have shown evidence of the existence of a form of diastolic dysfunction in which the late I_{Na} was not increased. Accordingly, no studies to date have demonstrated the treatment of diastolic dysfunction characterized by a lack of increased late I^{Na}.

Furthermore, no studies have demonstrated the existence of a form of diastolic dysfunction in which myo filament calcium sensitivity was increased. Accordingly, no studies have shown the treatment of diastolic dysfunction characterized by an increase in myofilament calcium sensitivity.

### SUMMARY OF THE INVENTION

Demonstrated herein for the first time are data which suggest that other sub-types of diastolic dysfunction do, in fact, exist. Also presented herein for the first time are data which demonstrate the treatment of these other diastolic dysfunction sub-types.

In this regard, the invention provides a method of treating diastolic dysfunction in a subject. The method comprises administering to the subject in an amount effective to treat the diastolic dysfunction a cardiac metabolic modifier.

In some embodiments, the diastolic dysfunction is diastolic dysfunction with preserved ejection fraction. In some embodiments, the diastolic dysfunction, e.g., diastolic dysfunction with preserved ejection fraction, is characterized by (i) a lack of increased late I_{Na} in cardiomyocytes, (ii) an increase in myofilament calcium sensitivity, or (iii) a combination thereof. Furthermore, in some embodiments, the subject does not suffer from a cardiac injury or structural heart disease, as further described herein.

Because diastolic dysfunction can lead to heart failure with preserved ejection fraction, the invention also provides a method of treating or preventing heart failure with preserved ejection fraction. The method comprises administering to the subject in an amount effective to treat or prevent the heart failure with preserved ejection fraction a cardiac metabolic modifier. In specific aspects, the heart failure with preserved ejection fraction is characterized by (i) a lack of increased late I_{Na} in cardiomyocytes, (ii) an increase in myofilament calcium sensitivity, or (iii) a combination thereof. In specific aspects, the subject does not suffer from a cardiac injury or structural heart disease, as further described herein.

The invention further provides a method of modulating (e.g., reducing) myofilament calcium sensitivity in a subject, comprising administering to the subject in an amount effective to modulate (e.g., reduce) myofilament calcium sensitivity a cardiac metabolic modifier. In some embodiments, the subject suffers from diastolic dysfunction, such as any of the sub-types of diastolic dysfunction described herein (e.g., diastolic dysfunction characterized by (i) a lack of increased late I_{Na} in cardiomyocytes, (ii) an increase in myofilament calcium sensitivity, or (iii) a combination thereof). Accordingly, the invention furthermore provides a method of treating a condition associated with or caused by increased myofilament calcium sensitivity in a subject. The method in exemplary aspects comprises administering to the subject in an amount effective to treat the condition a cardiac metabolic modifier.

The invention furthermore provides a method of treating acute decompensated heart failure in a subject in need thereof. In exemplary aspects, the method of treating acute decompensated heart failure comprises the step of administering to the subject a cardiac metabolic modifier in an amount effective to treat the acute decompensated heart failure.

### BRIEF DESCRIPTION OF THE DRAWINGS

FIG. 1. Representative echocardiographic assessments of LV diastolic function. Septal mitral annulus velocities interrogated with tissue Doppler imaging (TDI). The sham mouse has a larger E′ (early diastolic velocity), and smaller A′ (late diastolic velocity) than the hypertensive DOCA-salt mouse (upper panel). Treatment with ranolazine increased the ratio of E′ to A′ in the DOCA-salt mouse. Treatment of the sham mouse with ranolazine had little effect on mitral annulus velocities in the sham mouse. Sm (systolic septal mitral annulus velocity) was similar among all four groups.

FIG. 2. Invasive hemodynamic assessment of LV diastolic dysfunction. A and B: The end-diastolic pressure-volume relation (EDPVR) slope is steeper in DOCA-salt mice as compared to sham and ranolazine treated DOCA-salt mice. In panel A, the difference in the EDPVR in three representative mice from sham, DOCA-salt, and DOCA-salt treated mice are depicted. In panel B, the mean EDPVR is significantly greater in the DOCA-salt mice compared to sham and ranolazine treated sham mice. Additionally, treatment with ranolazine reduces the EDPVR in DOCA-salt mice to that of controls (n=8, *p<0.05 vs. all groups). C: Ranolazine shows a linear concentration dependent effect on the EDPVR in DOCA-salt mice with a Pearson correlation coefficient of 0.70 (n=6, p<0.05).

FIG. 3. Functional analysis of isolated cardiomyocytes. A: The normalized contraction of individual cardiomyocytes illustrating the difference in relaxation between a DOCA-salt mouse and a ranolazine treated DOCA-salt mouse. B: Fractional shortening of isolated cardiomyocytes paced at 0.5 Hz represented as the peak shortening divided by the baseline sarcomere length (n=10, p=NS). C: Time to 90% peak contraction in isolated cardiomyocytes (n=10, p=NS). D: Isolated cardiomyocytes from DOCA-salt mice have a prolonged relaxation constant (τ) compared to control animals. The addition of ranolazine to isolated DOCA-salt cardiomyocytes normalizes relaxation kinetics (n=10, *p<0.0001 vs. all groups).

FIG. 4. DOCA-salt mice have no difference in intracellular calcium cycling when compared to sham mice. B: The peak Ca2+ fluorescence in isolated cardiomyocytes loaded with the ratiometric fluorescent dye, Fura-2AM, and paced at 0.5 Hz (n=10, p=NS). C: The time to 90% peak Ca2+ fluorescence representing the rate of calcium entry into the cytosol (n=10, p=NS). D: The rate of relaxation measured as the time constant τ did not differ among groups (n=10, p=NS).

FIG. 5. DOCA-salt mice show no difference in late INa when compared to sham mice. A: Voltage-clamp studies show no increase in late INa in DOCA-salt myocytes with respect to sham (n=7, p=NS). Extracellular addition of ranolazine had little effect on late accumulated charge in both the DOCA-salt and sham groups (n=4, p=NS). Peak INa was similar among the four groups. B: Graph of the ratio of the mean accumulated late Na+ charge to the mean accumulated total Na+ charge during an activating voltage step.

FIG. 6. Resting sarcomere length of cardiomyocytes and pCa-tension relations in skinned fiber bundles. A: The mean diastolic sarcomere length was significantly shorter in the DOCA-salt cardiomyocytes compared to the sham. The addition of ranolazine to the DOCA-salt cardiomyocytes significantly lengthened resting sarcomeres, but had no effect on sham cardiomyocytes (n=12, *p<0.0001 vs. all groups). B: The mean steady-state isometric tension of skinned fiber bundles plotted as a function of pCa. C: pCa-tension relations normalized to maximum tension. DOCA-salt fibers (pCa50=6.1+/−0.02; Hill n=3.42+/−0.30) demonstrate a significant increase in mean Ca2+ sensitivity (n=6, p<0.05) as compared to shams without DOCA treatment (pCa50=6.0+/−0.01; Hill n=3.80+/−0.61) shams with DOCA treatment (pCa50=6.0+/−0.01; Hill n=3.91+/−0.50) and fibers from DOCA mice and treated with ranalozine. (pCa50=6.0+/−0.03; Hill n=3.71+/−1.0) D: Plot of tension normalized to maximum steady-state isometric tension as a function of pCa.

FIG. 7. Invasive hemodynamic assessment of LV diastolic dysfunction. A: Representative pressure-volume loops with vena cava occlusion for DOCA-salt and DOCA-salt+ranolazine groups. B: The EDPVR slope is greater in DOCA-salt vs. sham mice (P<0.05) and is improved with ranolazine treatment (P<0.05, vs. DOCA-salt). The mean EDPVR is significantly greater in the DOCA-salt mice compared to sham and ranolazine-treated mice. Additionally, treatment with ranolazine reduces the EDPVR in DOCA-salt mice to that of controls (n=8, *p<0.05 vs. all groups). C: LV contractility assessed by the end-systolic pressure-volume relation (ESPVR) slope (P=NS) and the volume axis intercept (Vo, P=NS) are similar in sham, DOCA-salt and DOCA-salt+ranolazine groups. D: Comparisions of LV end-diastolic pressure (LVEDP) for sham (3.2±0.3 mmHg, P<0.05 vs. DOCA-salt), DOCA-salt (5.1±0.9 mmHg), and DOCA-salt+ranolazine (3.5±0.4 mmHg, P<0.05 vs. DOCA-salt) groups. E: Ranolazine shows an inverse concentration dependent effect on the EDPVR in DOCA-salt mice with a Pearson correlation coefficient of 0.70 (n=6, p<0.05).

FIG. 8. Functional analysis of isolated cardiomyocytes. A: Fractional shortening of isolated cardiomyocytes paced at 0.5 Hz at 37° C. represented as the peak shortening divided by the baseline sarcomere length (n=12, p=NS). B: Time to 90% peak contraction in isolated cardiomyocytes (n=12, p=NS). C: Isolated cardiomyocytes from DOCA-salt mice have a prolonged relaxation constant (τ) compared to control animals. The addition of ranolazine to isolated DOCA-salt cardiomyocytes normalizes relaxation kinetics (n=12, *p<0.0001 vs. all groups). D: The mean diastolic sarcomere length was significantly shorter in the DOCA-salt cardiomyocytes compared to the sham. The addition of ranolazine to the DOCA-salt cardiomyocytes significantly lengthened resting sarcomeres, but had no effect on sham cardiomyocytes (n=12, *p<0.0001 vs. all groups).

FIG. 9. DOCA-salt mice have no difference in intracellular calcium cycling when compared to sham mice. B: The peak Ca²⁺ fluorescence in isolated cardiomyocytes loaded with the ratiometric fluorescent dye, Fura-2AM, and paced at 0.5 Hz (n=12, p=NS). C: The time to 90% peak Ca²⁺ fluorescence representing the rate of calcium entry into the cytosol (n=12, p=NS). D: The rate of relaxation measured as the time constant τ did not differ among groups (n=12, p=NS).

FIG. 10. DOCA-salt mice show no difference in late I_{Na} when compared to sham mice. A: Voltage-clamp studies show no increase in late I_{Na} in DOCA-salt myocytes with respect to sham (n=7, p=NS). Extracellular addition of ranolazine had little effect on late accumulated charge in both the DOCA-salt and sham groups (n=4, p=NS). B: Graph of the ratio of the mean accumulated late Na⁺ charge to the mean accumulated total Na⁺ charge during an activating voltage step.

FIG. 11. Myofilament Ca²⁺ responsiveness in skinned fiber bundles isolated from DOCA-salt and sham hearts with and without ranolazine. A-D: The mean steady-state isometric tension of skinned fiber bundles is plotted as a function of pCa. DOCA-salt fibers demonstrate a significant (p<0.05) increase in maximum tension (44.51±0.55 mN/mm²; n=6) compared to shams (39.7±0.79 mN/mm²; n=6) and DOCA-salt fibers treated with ranolazine (36.03±1.50 mN/mm²). DOCA-salt fibers demonstrate a significant (p<0.05) increase in mean Ca²⁺ sensitivity (pCa₅₀=6.09±0.01; n=6) compared to shams (pCa₅₀=6.02±0.01; n=6). Treatment with ranolazine normalized these changes. E: pCa-tension relations normalized to maximum tension. DOCA-salt fibers (pCa₅₀=6.1±0.02; Hill n=3.42±0.30) demonstrate a significant increase in mean Ca²⁺ sensitivity (n=6, *p<0.05) as compared to shams without DOCA treatment (pCa₅₀=6.0±0.01; Hill n=3.80±0.61) shams with DOCA treatment (pCa₅₀=6.0±0.01; Hill n=3.91±0.50) and fibers from DOCA mice and treated with ranalozine (pCa₅₀=6.0±0.03; Hill n=3.71±1.0).

FIG. 12. A comparison in the post-translational modifications of the myofilaments from sham and DOCA-salt hearts. A: Western blot analysis demonstrating detection of glutathionylated proteins when comparing myofilament samples from sham and DOCA-salt treated hearts. The blots were also analyzed for myosin binding protein C, indicating that this protein was the major modification. Note that one pair of comparisons was removed from the gel. B: Quantification of the difference between glutathionylated proteins (GSH) normalized to myosin binding protein C (MyBP-C) in sham and DOCA-salt myofilaments. (*p<0.05, n=3).

FIG. 13. Representative echocardiographic assessments of LV diastolic function. Septal mitral annulus velocities interrogated with tissue Doppler imaging (TDI). The sham mouse has a higher E′ (early diastolic velocity), and lower A′ (late diastolic velocity) than the hypertensive DOCA-salt mouse (upper panel). Treatment with ranolazine increased the ratio of E′ to A′ in the DOCA-salt mouse. Treatment of the sham mouse with ranolazine had little effect on mitral annulus velocities in the sham mouse. Sm (systolic septal mitral annulus velocity) was similar among all four groups.

FIG. 14. Effects of rate on relaxation and resting sarcomere length in isolated cardiomyocytes. A: DOCA-salt cardiomyocytes paced at 1 Hz show a significantly prolonger relaxation time (τ) compared with the other groups. B: At 2 Hz, the effect on relaxation was similar to slower pacing with a significantly slowed relaxation time. Ranolazine improved relaxations times at both frequencies. C and D: DOCA-salt cardiomyocytes demonstrated a significantly shorter sarcomere length compared to the other groups and both 1 and 2 Hz. Ranolazine lengthened sarcomere length at both frequencies. *p<0.05 DOCA-salt vs. all other groups.

FIG. 15. Effect of rate on resting Ca²⁺ levels and Ca²⁺ extrusion from isolated cardiomyocytes. A and B: There is no significant difference in resting Ca²⁺ levels at rest among the four groups at 1 and 2 Hz. C and D: Additionally, there were no significant differences in the rate of Ca²⁺ extrusion from the isolated cardiomyocytes at rates of 1 and 2 Hz.

FIG. 16. Chemo-mechanical transduction in fiber bundles treated with ranolazine from sham and DOCA-salt treated hearts. The relationship between ATPase rate and tension development. Tension cost was determined as the slope of the relationship between tension and ATPase activity. *P<0.05 for DOCA-salt+ranolazine compared to DOCA-salt+DMSO. ^{†}P<0.05 for DOCA-salt+DMSO compared to Sham+DMSO. See text for details.

FIG. 17. Representative ProQ (A) and Coomassie R-250 gels (B) showing phosphorylation states of various myofilament proteins of fibers treated acutely with either ranolazine or DMSO.

FIG. 18. Representative comparisons of the post-translational modifications of the myofilaments from sham and DOCA-salt hearts. A: Comparison of myosin binding protein C (MyBP-C), B: Troponin I (TnI), and C: myosin light chain 2 (MLC₂) phosphorylations in fiber bundles from sham and DOCA-salt hearts. There were no significant differences. Values are given as means±SEM for 6-9 determinations.

FIG. 19. Representative ProQ (A) and Coomassie R-250 gels (B) showing phosphorylation states of various myofilament proteins of hearts from DOCA-salt or sham mice after or without chronic administration of ranolazine.

FIG. 20. A: Representative gels showing changes in S-glutathionylation of myosin binding protein-C. B: Comparison of myosin binding protein C (MyBP-C)S-glutathionylation in hearts from DOCA-salt or sham mice after or without chronic administration of ranolazine. Values are given as means±SEM for 5 determinations.

FIG. 21. Phase-plane diagram between the fura-2 ratio (340/380) and sarcomere length during the entire contraction-relaxation cycle between sham and DOCA-salt cardiomyocytes in the presence or absence of ranolazine.

FIG. 22. The normalized contraction of individual cardiomyocytes illustrating the difference in relaxation between a DOCA-salt mouse and a ranolazine treated DOCA-salt mouse.

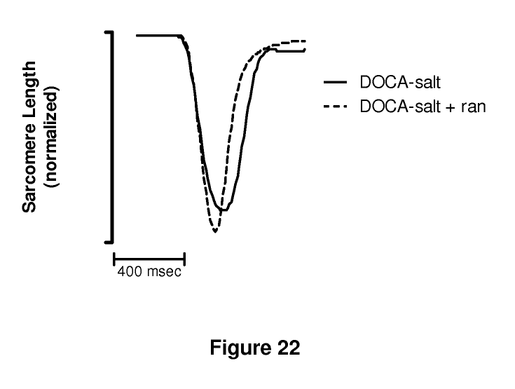

### DETAILED DESCRIPTION OF THE INVENTION

Diastolic Dysfunction

The invention provides a method of treating diastolic dysfunction in a subject, comprising administering a cardiac metabolic modifier to the subject in an amount effective to treat the diastolic dysfunction. In exemplary aspects, the cardiac metabolic modifier lowers myofilament calcium sensitivity, e.g., lowers calcium sensitivity of myofilaments of cardiac muscle. In exemplary aspects, the cardiac metabolic modifier binds to myofilaments of cardiac muscle. In exemplary aspects, the cardiac metabolic modifier comprises a structure of Formula I, as described herein. See, e.g., the section entitled herein as “Cardiac Metabolic Modifiers.”

As used herein, the term “diastolic dysfunction” refers to a condition in which abnormalities in mechanical function are present during diastole and which can occur in the presence or absence of heart failure and can co-exist with or without abnormalities in systolic function (Zile et al., JACC 41: 1519-1522 (2003)). Accordingly, with regard to the invention disclosed herein, the diastolic dysfunction in some embodiments is diastolic dysfunction with preserved ejection fraction, which is also known as, diastolic dysfunction with preserved systolic function, diastolic dysfunction without systolic dysfunction, and diastolic dysfunction with preserved left ventricular function. As used herein, the term “preserved ejection fraction” refers to a left ventricular ejection fraction which is greater than or about 45%, e.g., greater than or about 50%. In some aspects, the preserved ejection fraction is one which is greater than or about 50%.

In some embodiments, the diastolic dysfunction is characterized by measurement of left ventricle (LV) pressure, volume, wall thickness, calculations that reflect the process of active relaxation (the rate of isovolumic LV pressure and LV filling) and calculations that reflect passive stiffness (chamber compliance and myocardial viscoelastic stiffness) (Zile et al., (2010), supra).

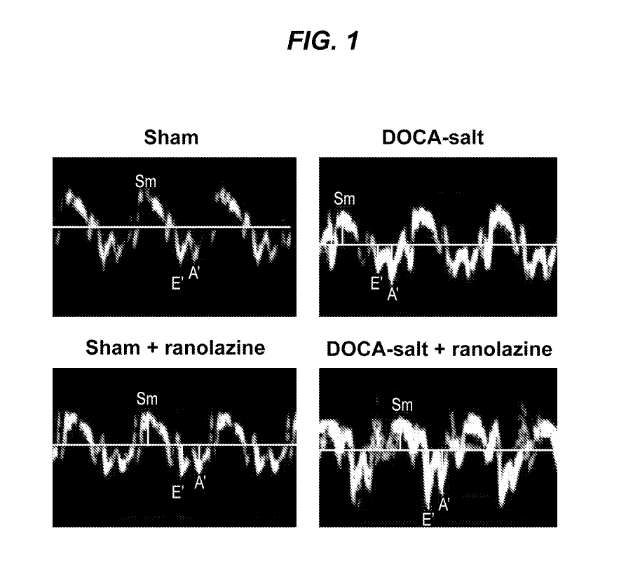

In some embodiments, the diastolic dysfunction is evidenced via echocardiography, as described in Silberman et al., Circulation 121: 519-528 (2010) and as described in the EXAMPLES section set forth below. In some aspects, LV tissue Doppler and mitral valve in-flow velocity are measured by echocardiography. In some aspects, the diastolic dysfunction is characterized by a late diastolic velocity (A′) which is higher than the early diastolic velocity (E′), as shown by tissue Doppler imaging (TDI). See, for example, FIG. 1 and the brief description thereof.

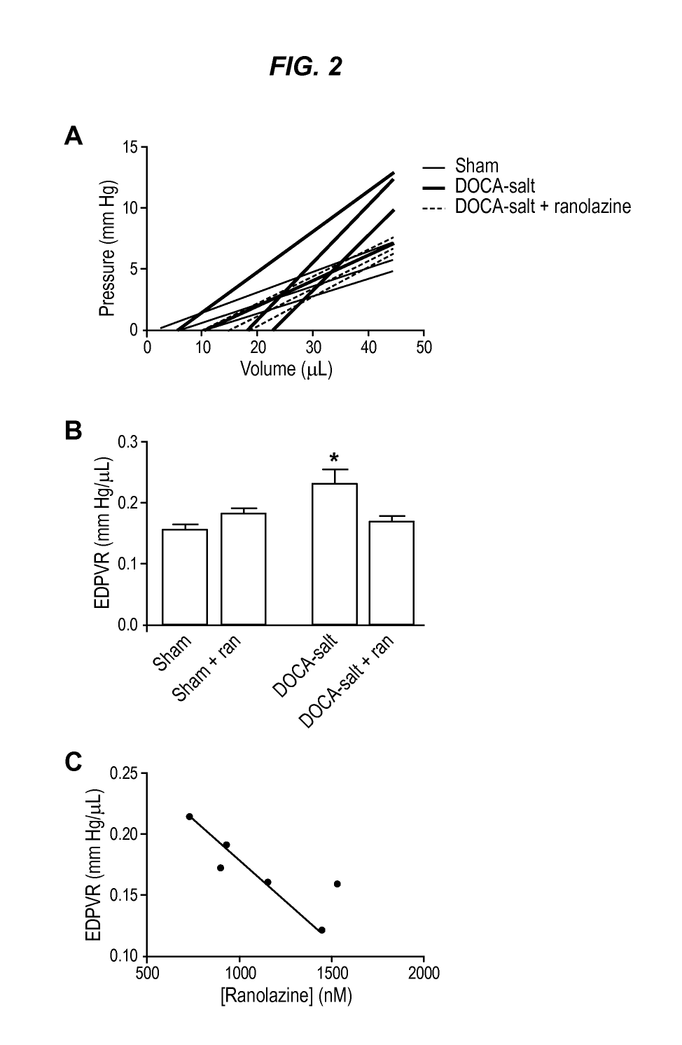

In alternative or additional embodiments, the diastolic dysfunction is characterized through magnetic resonance imaging (MRI) or by cardiac catheterization and measurement of LV end diastolic pressure and systolic function. In some aspects, the diastolic dysfunction is characterized by an end-diastolic pressure-volume relation (EDPVR) slope which is steeper than the EDPVR slope of a corresponding control (e.g., which does not suffer from diastolic dysfunction). In some aspects, the diastolic dysfunction is characterized by a slope of the line which correlates end-diastolic pressure (mm Hg) to end-diastolic volume (μL) which is steeper than the slope of the line which correlates end-diastolic pressure (mm Hg) to end-diastolic volume (μL) of a corresponding control (e.g., which does not suffer from diastolic dysfunction). See, for example FIGS. 2A-2C, the brief description thereof, and the EXAMPLES section set forth below. In some embodiments, the diastolic dysfunction is characterized by an EDPVR slope which is increased by about 25% or more (e.g., about 30% or more, about 35% or more about 40% or more, about 45% or more, about 50% or more), as compared to the EDPVR slope of a corresponding control (e.g., which does not suffer from diastolic dysfunction). In some embodiments, the diastolic dysfunction is characterized by an EDPVR slope which is increased by about 25% to about 60%, by about 30% to about 55%, about 35% to about 50%, or about 40% to about 45% (e.g., about 40%, about 41%, about 42%, about 43%, about 44%, about 45%), as compared to the EDPVR slope of a corresponding control (e.g., which does not suffer from diastolic dysfunction). In exemplary embodiments, the diastolic dysfunction is characterized by an EDPVR slope which is about 0.15 or higher, e.g., about 0.16 or higher, about 0.17 or higher, about 0.19 or higher, about 0.20 or higher, about 0.21 or higher, about 0.22 or higher, about 0.23 or higher, about 0.24 or higher, about 0.25 or higher, about 0.26 or higher, about 0.27 or higher, about 0.28 or higher, about 0.29 or higher, about 0.30 or higher.

In some aspects, the diastolic dysfunction is characterized through one or more of the invasive and non-invasive procedures described in the EXAMPLES section set forth below.

In some embodiments, the diastolic dysfunction is not diastolic dysfunction which is induced by administration of a toxin or by ischemia, ischemia-reperfusion, or coronary artery-occlusion-reperfusion. In some aspects, the diastolic dysfunction is not diastolic dysfunction which is induced by administration of sea anemone toxin ATX-II described in Sossalla et al., J Molec Cell Cardiology 45: 32-43 (2008) or in Fraser et al., J Molec Cell Cardiology 41: 1031-1038 (2006). In some aspects, the diastolic dysfunction is not induced by an ischemic metabolite, e.g., palmitoyl-L-carnitine, as described in Wu et al., J Pharmacology Expmtl Therapeutics 330: 550-557 (2009). In some aspects, the diastolic dysfunction is not diastolic dysfunction which is induced by administration of hydrogen peroxide. In some aspects, the diastolic dysfunction is not induced by stimulating muscle at a basal stimulation frequency followed by stretching until maximum steady-state twitch force is achieved, as described in Sossalla et al. (2008), supra. In some aspects, the diastolic dysfunction is not induced by E-4031, 4-aminopyridine, and BaCl₂.

In some embodiments, the diastolic dysfunction co-exists with another medical condition. For example, as further discussed herein, in some aspects, the diastolic dysfunction co-exists with hypertension or a metabolic disease (e.g., diabetes, obesity).

In some embodiments, the diastolic dysfunction does not co-exist with a cardiac injury or structural heart disease. Cardiac injuries and structural heart diseases that do not co-exist with the diastolic dysfunction are further described herein under Subjects. In some aspects, the diastolic dysfunction does not co-exist with ischemic heart disease, chronic stable angina, chronic angina. In some aspects, the diastolic dysfunction does not co-exist with ischemia, ischemia-reperfusion or coronary artery occlusion-reperfusion, ischemic heart disease, myocardial injury, myocardial toxicity, myocardial infarction, congenital heart lesion, valvular stenosis or valvular regurgitation, coronary artery disease, chronic angina, chronic stable angina, arrhythmias.

In further aspects of the invention disclosed herein, the diastolic dysfunction is characterized by (i) a lack of increased late I_{Na} in cardiomyocytes, (ii) an increase in myofilament calcium sensitivity, or (iii) a combination thereof.

Late I_{Na} in Cardiomyocytes

Late I_{Na} and methods of measuring late I_{Na} are described herein, as well, as in Fraser et al., J Molecular and Cell Cardiology 41: 1031-1038 (2006), which describes late I_{Na} as a sustained/persistent influx of Na+, due to slowed or incomplete inactivation of voltage-gated sodium channels in the myocardium. In some sub-types of diastolic dysfunction, the diastolic dysfunction is hallmarked by an increase in the late I_{Na}. In some embodiments of the present disclosures, this sub-type of diastolic dysfunction is different from the diastolic dysfunction treatable by the methods described herein, inasmuch as, in some embodiments, the diastolic dysfunction treatable by the methods described herein lack a substantial increase in late I_{Na}. In some aspects, the diastolic dysfunction is characterized by a late I_{Na} which is substantially similar to the late I_{Na}. of subjects not suffering from diastolic dysfunction. In some aspects, the diastolic dysfunction is characterized by a ratio of the mean accumulated late Na+ charge to the mean accumulated total Na+ charge during an activating voltage step which is substantially similar to the ratio of the mean accumulated late Na+ charge to the mean accumulated total Na+ charge during an activating voltage step in a corresponding control (e.g., in a subject that does not suffer from diastolic dysfunction). In some aspects, the diastolic dysfunction is characterized by a ratio of the mean accumulated late Na+ charge to the mean accumulated total Na+ charge during an activating voltage step which differs from the ratio of the mean accumulated late Na+ charge to the mean accumulated total Na+ charge during an activating voltage step of a corresponding control (e.g., in a subject that does not suffer from diastolic dysfunction) by no more than 5% (e.g., no more than 4%, no more than 3%, no more than 2%, no more than 1%, no more than 0.75%, no more than 0.5%, no more than 0.4%, nor more than 0.3%, no more than 0.25%, no more than 0.2%, no more than 0.15%, no more than 0.1%, no more than 0.01%, no more than 0.001%). In some aspects, the late I_{Na} is measured as essentially described in the EXAMPLES section set forth below. Accordingly, in some aspects, integrated late I_{Na} is measured starting at 5% of peak current and ending 40 ms after depolarization.

In some embodiments, the late I_{Na} in normal conditions (e.g., in subjects not suffering from diastolic dysfunction) constitutes only about 1% of peak, or total I_{Na}. See, for example, Dobesh et al., Pharmacotherapy, 27:1659-1675 (2007). Accordingly, in some aspects herein, a “lack of increased late I_{Na}” is a late I_{Na} which is about 1% of peak, or total I_{Na}. In this regard, the diastolic dysfunction in some aspects is characterized by a late I_{Na} which is about 1% of peak, or total I_{Na}. In some aspects, the diastolic dysfunction is characterized by a late I_{Na} which is about 1.5% to about 0.5% of peak or total I_{Na}. In exemplary aspects, the diastolic dysfunction is characterized by a late I_{Na} which is about 1.25% to about 0.75% of peak or total I_{Na}. In some aspects, diastolic dysfunction is characterized by a late I_{Na} which is about 0.9% to about 1.1% or about 0.8% to about 1.2% of peak or total I_{Na}.

Myofilament Calcium Sensitivity

For contraction to occur, cardiac muscle, which is composed of alternating segments of thin and thick myofilaments, requires an inward flux of extracellular calcium ions through L-type calcium channels. In some embodiments, the diastolic dysfunction is characterized by an increased myofilament calcium sensitivity. In some aspects, the myofilament calcium sensitivity is indexed by pCa₅₀, which is the logarithm of the calcium concentration at which myofilaments generate 50% of the total maximum tension. In some aspects, the diastolic dysfunction is characterized by a pCa₅₀ which is increased by about 0.1% to about 5%, about 0.5% to about 2%, about 1% to about 1.5%, as compared to the pCa₅₀ of myofilaments of subjects not suffering from diastolic dysfunction. In some aspects, the diastolic dysfunction is characterized by a pCa₅₀ which is increased by about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about, 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5%, as compared to the pCa₅₀ of myofilaments of subjects not suffering from diastolic dysfunction.

In some aspects, the myofilament calcium sensitivity is represented by the pCa-tension relations normalized to maximum tension, and the diastolic dysfunction is characterized by a pCa-tension relations normalized to maximum tension which is increased by about 0.1% to about 5%, about 0.5% to about 2%, about 1% to about 1.5%, as compared to the pCa-tension relations normalized to maximum tension of myofilaments of subjects not suffering from diastolic dysfunction. In some aspects, the diastolic dysfunction is characterized by a pCa-tension relations normalized to maximum tension which is increased by about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about, 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5%, as compared to the pCa₅₀ of myofilaments of subjects not suffering from diastolic dysfunction.

Methods of measuring myofilament calcium sensitivity are known in the art (e.g., Varian et al., Am J Physiol Heart Circ Physiol 290:H2092-H2097 (2006)) and are described in the EXAMPLES section set forth herein.

Additional Characteristics

In yet further aspects, the diastolic dysfunction is characterized by (i) a lack of change in calcium cycling or calcium handling in cardiomyocytes; (ii) a lack of change in calcium concentration in resting myocytes; (iii) a decrease in sarcomere length in resting myocytes; (iv) an increase in diastolic tension; or (v) a combination thereof.

Intracellular Calcium Cycling or Calcium Handling and Calcium Concentration in Resting Myocytes

When there is an increase in late I_{Na}, the influx of Ca²⁺ through the reverse mode of the Na⁺—Ca²⁺ exchanger increases, resulting in an overload or increase in intracellular Ca²⁺. In some embodiments herein, the diastolic dysfunction treated by the invention is characterized by substantially unchanged calcium cycling and/or by substantially unchanged intracellular calcium concentrations, as compared to the calcium cycling and/or intracellular calcium concentrations of myocytes of subjects not afflicted with diastolic dysfunction.

In some aspects, the calcium cycling or calcium flux in the myocardiocytes are similar to those found in the myocardiocytes of subjects not suffering from diastolic dysfunction. In some aspects, the baseline Ca²⁺, the peak Ca²⁺, the rate of Ca²⁺ release, and/or the rate of intracellular Ca²⁺ egress are considered normal or substantially unchanged. In some aspects, the calcium concentration of resting myocytes are substantially unchanged, as compared to the calcium concentration of resting myocytes of subjects not suffering from diastolic dysfunction. By “substantially unchanged” as used herein means that the parameter (e.g., intracellular calcium (Ca²⁺) cycling, baseline Ca²⁺, the peak Ca²⁺, the rate of Ca²⁺ release, the rate of intracellular Ca²⁺ egress, calcium concentration of resting myocytes) differs by no more than about 10% (e.g., about 10% or less, about 9% or less, about 8% or less, about 7% or less, about 6% or less, about 5% or less, about 4% or less, about 3% or less, about 2% or less, about 1% or less, about 0.9% or less, about 0.8% or less, about 0.7% or less, about 0.6% or less, about 0.5% or less, about 0.4% or less, about 0.3% or less, about 0.2% or less, about 0.1% or less), the parameter exhibited by myocytes of a subject not suffering from diastolic dysfunction.

Methods of measuring calcium cycling are known in the art and include, for example, the calcium experiments described in the EXAMPLES section set forth below.

Unloaded Sarcomere Length in Resting Myocytes

In some embodiments, the diastolic dysfunction is characterized by a decreased unloaded sarcomere length in isolated resting myocytes. In some aspects, the diastolic dysfunction is characterized by a decreased baseline sarcomere length in isolated resting myocytes or by a decreased mean diastolic sarcomere length in isolated resting myocytes. In some aspects, the decrease in unloaded sarcomere length, baseline sarcomere length, or mean diastolic sarcomere length is a decrease of about 5% (e.g., about 4.5%, about 4%, about 3.5%, about 3.0%, about 2.5%, about 2%, about 1.5%, about 1%, about 0.5%) of the unloaded sarcomere length, baseline sarcomere length, or mean diastolic sarcomere length of isolated resting myocytes of subjects not suffering from diastolic dysfunction. In some aspects, the decrease in unloaded sarcomere length, baseline sarcomere length, or mean diastolic sarcomere length in isolated resting myocytes is measured by the procedures described in the EXAMPLES section set forth below.

Diastolic Tension

In some embodiments, the diastolic tension is increased, as compared to the diastolic tension of subjects not suffering from diastolic dysfunction. In some aspects, the diastolic tension is increased by about 50% or less (e.g., about 25% or less, about 15% or less, about 10% or less, about 5% or less, about 4% or less, about 2.5% or less, about 1% or less, about 0.5% or less). Methods of measuring diastolic tension are known in the art (e.g., Sossalla et al., (2008), supra and in the EXAMPLES section set forth herein.

Heart Failure with Preserved Ejection Fraction or with Preserved Left Ventricular Function

Heart failure with preserved ejection fraction, which is also known as, heart failure with preserved systolic function, heart failure without systolic dysfunction, and heart failure with preserved left ventricular function, is a clinical condition in which the subject exhibits a preserved ejection fraction (e.g., an ejection fraction which is greater than or about 45%, or greater than or about 50%) along with signs and/or symptoms of heart failure. The signs and symptoms of heart failure in some embodiments include dyspnea, fatigue, exercise intolerance, jugular venous distension, pulmonary rales, peripheral edema, pulmonary vascular redistribution, interstitial edema, pleural effusions.

Because diastolic dysfunction can lead to heart failure with preserved ejection fraction, the invention also provides a method of treating or preventing heart failure with preserved ejection fraction. The method comprises administering a cardiac metabolic modifier to the subject in an amount effective to treat or prevent the heart failure with preserved ejection fraction. In exemplary aspects, the cardiac metabolic modifier lowers myofilament calcium sensitivity, e.g., lowers calcium sensitivity of myofilaments of cardiac muscle. In exemplary aspects, the cardiac metabolic modifier binds to myofilaments of cardiac muscle. In exemplary aspects, the cardiac metabolic modifier comprises a structure of Formula I, as described herein. See, e.g., the section entitled herein as “Cardiac Metabolic Modifiers.”

In specific aspects, the heart failure with preserved ejection fraction is characterized by (i) a lack of increased late I_{Na} in cardiomyocytes, (ii) an increase in myofilament calcium sensitivity, or (iii) a combination thereof. In further embodiments, the heart failure with preserved ejection fraction is characterized by (i) a lack of change in calcium cycling or calcium handling in cardiomyocytes; (ii) a lack of change in calcium concentration in resting myocytes; (iii) a decrease in sarcomere length in resting myocytes; (iv) an increase in diastolic tension; or (v) a combination thereof. Such characteristics are described herein and are applicable to the methods of treating or preventing heart failure with preserved ejection fraction.

In some embodiments, the heart failure is a Class III or Class IV heart failure, as defined by the New York Heart Association (NYHA). In some aspects, the heart failure is not a Class I or Class II heart failure, as defined by the NYHA. See, for example, The Criteria Committee of the New York Heart Association. Nomenclature and Criteria for Diagnosis of Diseases of the Heart and Great Vessels. 9th ed. Boston, Mass.: Little, Brown & Co; 1994:253-256. Accordingly, in some embodiments, the heart failure is heart failure in which the subject presents the symptoms of NYHA Class III or Class IV as indicated in the following table. In some embodiments, the heart failure is heart failure in which the subject does not present any of the symptoms of NYHA Class I or II as indicated in the following table.

| NYHA | |
|---|---|
| Class | Symptoms |
| I | No symptoms and no limitation in ordinary physical activity, |
| | e.g. shortness of breath when walking, climbing stairs etc. |
| II | Mild symptoms (mild shortness of breath and/or angina) and |
| | slight limitation during ordinary activity. |
| III | Marked limitation in activity due to symptoms, even during less- |
| | than-ordinary activity, e.g. walking short distances (20-100 m). |
| | Comfortable only at rest. |
| IV | Severe limitations. Experiences symptoms even while at rest. |
| | Mostly bedbound patients. |

Acute Decompensated Heart Failure

The invention furthermore provides a method of treating acute decompensated heart failure in a subject. In exemplary aspects, the method of treating acute decompensated heart failure comprises the step of administering to the subject a cardiac metabolic modifier in an amount effective to treat the acute decompensated heart failure in the subject. In exemplary aspects, the cardiac metabolic modifier lowers myofilament calcium sensitivity, e.g., lowers calcium sensitivity of myofilaments of cardiac muscle. In exemplary aspects, the cardiac metabolic modifier binds to myofilaments of cardiac muscle. In exemplary aspects, the cardiac metabolic modifier comprises a structure of Formula I, as described herein. See, e.g., the section entitled herein as “Cardiac Metabolic Modifiers.”

In exemplary aspects, the method of treating acute decompensated heart failure provided herein comprises administration of one or more immediate release dosages of the cardiac metabolic modifier. In exemplary aspects, the method of treating acute decompensated heart failure provided herein comprises administration of one or more sustained or extended release dosages of the cardiac metabolic modifier.

With regard to the methods of treating acute decompensated heart failure provided herein, the cardiac metabolic modifier in some aspects, is intravenously administered to the subject, orally administered to the subject, or both. The intravenous administration may occur at the same time of oral administration. Alternatively, the intravenous administration may occur before and/or after the oral administration. In exemplary aspects, the method comprises multiple intravenous administrations an/or oral administrations of the cardiac metabolic modifier, and the administrations occur in a particular order and at specific dosages.

In exemplary aspects, the method comprises the steps of intravenously administering to the subject a first dose of the cardiac metabolic modifier, intravenously administering to the subject a second dose of the cardiac metabolic modifier, and orally administering to the subject a third dose of the cardiac metabolic modifier. In exemplary aspects, (i) the first dose is administered to the subject over a time period of about 45 minutes to about 90 minutes, (e.g., about 45 minutes, about 50 minutes, about 55 minutes, about 60 minutes, about 65 minutes, about 70 minutes, about 75 minutes, about 80 minutes, about 85 minutes, about 90 minutes, about 50 to about 85 minutes, about 55 to about 80 minutes), (ii) the second dose is administered to the subject over a time period of about 36 hours to about 76 hours (e.g., about 36 hours, about 42 hours, about 48 hours, about 54 hours, abhout 60 hours, about 66 hours, about 72 hours, about 42 to about 72 hours, about 48 to about 66 hours, about 54 hours to about 60 hours), (iii) the third dose is administered to the subject twice daily, or (iv) a combination thereof.

In exemplary aspects, the methods provided herein comprise administration of the cardiac metabolic modifier at more than one dose, and the doses depend on whether or not the subject is taking another therapeutic agent. For example, if the subject is taking a CYP3A inhibitor, then the dose(s) of the cardiac metabolic modifier may be decreased, as compared to the dose(s) of the cardiac metabolic modifier administered to a subject not taking a CYP3A inhibitor. For example, (i) the first dose is within a range of about 180 mg/h to about 220 mg/h (e.g., about 185 mg/h to about 215 mg/h, about 180 mg/h to about 210 mg/h, about 185 to about 205 mg/h, about 190 mg/h to about 200 mg/h), (ii) the second dose is within a range of about 30 mg/h to about 50 mg/h (e.g., about 30 mg/h, about 35 mg/h, about 40 mg/h, about 45 mg/h, about 50 mg/h), if the subject is taking a CYP3A inhibitor, or is within a range of about 60 mg to about 100 mg (e.g., about 60 mg, about 70 mg, about 80 mg, about 90 mg, about 100 mg), if the subject is not taking a CYP3A inhibitor, (iii) each administration of the third dose is within a range of about 350 mg to about 650 mg (e.g., about 400 mg to about 600 mg, about 450 mg to about 550 mg), if the subject is taking a CYP3A inhibitor, or is within a range of about 900 mg to about 1100 mg (e.g., about 950 to about 1050 mg, about 1000 to about 1100 mg), if the subject is not taking a CYP3A inhibitor, or (iv) a combination thereof. In exemplary aspects, the CYP3A inhibitor is verapamil, diltiazem, or erythromycin

Modulation of Myofilament Calcium Sensitivity

In addition to the treatment and prevention methods provided herein, the invention additionally provides a method of modulating myofilament calcium sensitivity in a subject. The method comprises administering a cardiac metabolic modifier to the subject in an amount effective to modulate myofilament calcium sensitivity. In some aspects, the method reduces the myofilament calcium sensitivity. In exemplary aspects, the method modulates (e.g., lowers) the myofilament calcium sensitivity in the heart of the subject. For example, the method may lower the calcium sensitivity of myofilaments of cardiac muscle.

In exemplary aspects, the cardiac metabolic modifier lowers myofilament calcium sensitivity, e.g., lowers calcium sensitivity of myofilaments of cardiac muscle. In exemplary aspects, the cardiac metabolic modifier binds to myofilaments of cardiac muscle.

In exemplary aspects, the cardiac metabolic modifier comprises a structure of Formula I, as described herein.

In some aspects, the myofilament calcium sensitivity is indexed by pCa₅₀, which is the logarithm of the calcium concentration at which myofilaments generate 50% of the total maximum tension. In some aspects, the pCa₅₀ is reduced by about 0.1% to about 5%, about 0.5% to about 2%, about 1% to about 1.5% upon administration of the cardiac metabolic modifier or as compared to the pCa₅₀ before administration of the cardiac metabolic modifier. In some aspects, the pCa₅₀ is reduced by about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about, 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5% upon administration of the cardiac metabolic modifier or as compared to the pCa₅₀ before administration of the cardiac metabolic modifier.

In some aspects, the myofilament calcium sensitivity is represented by the pCa-tension relations normalized to maximum tension, and the method reduces the pCa-tension relations normalized to maximum tension by about 0.1% to about 5%, about 0.5% to about 2%, about 1% to about 1.5% upon administration of the cardiac metabolic modifier or as compared to the pCa-tension relations normalized to maximum tension before administration of the cardiac metabolic modifier. In some aspects, the pCa-tension relations normalized to maximum tension is reduced by about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about, 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5% upon administration of the cardiac metabolic modifier or as compared to the pCa-tension relations normalized to maximum tension before administration of the cardiac metabolic modifier.

In some embodiments, the subject suffers from diastolic dysfunction, such as any of the sub-types of diastolic dysfunction described herein. See, for example, the section entitled Diastolic Dysfunction.

Because increased myofilament calcium sensitivity is associated with or causes a number of medical conditions, including, for example, cardiac diseases (e.g., hypertrophic cardiomyopathy), it is postulated that reducing myofilament calcium sensitivity may effectively treat a condition associated with or caused by increased myofilament calcium sensitivity in a subject. Accordingly, the invention furthermore provides a method of treating a condition associated with or caused by increased myofilament calcium sensitivity in a subject. The method comprises administering a cardiac metabolic modifier to the subject in an amount effective to treat the condition. In some embodiments, the subject suffers from diastolic dysfunction, such as any of the sub-types of diastolic dysfunction described herein. See, for example, the section entitled Diastolic Dysfunction. In exemplary aspects, the cardiac metabolic modifier which is administered to the subject lowers myofilament calcium sensitivity, e.g., lowers calcium sensitivity of myofilaments of cardiac muscle. In exemplary aspects, the cardiac metabolic modifier binds to myofilaments of cardiac muscle. In exemplary aspects, the cardiac metabolic modifier comprises a structure of Formula I, as described herein. See, e.g., the section entitled herein as “Cardiac Metabolic Modifiers.”

The terms “treat,” “prevent,” “reduce” and “increase” as well as words stemming therefrom, as used herein, do not necessarily imply 100% or complete treatment, prevention, reduction, or increase. Rather, there are varying degrees of treatment, prevention, reduction, or increase of which one of ordinary skill in the art recognizes as having a potential benefit or therapeutic effect. In this respect, the methods of the present disclosures can provide any amount of any level of treatment of diastolic dysfunction, e.g., diastolic dysfunction with preserved ejection fraction, treatment or prevention of heart failure with a preserved ejection fraction in a subject. Furthermore, the treatment or prevention provided by the method of the present disclosures can include treatment or prevention of one or more conditions or symptoms of the disease, e.g., diastolic dysfunction with preserved ejection fraction, heart failure with preserved ejection fraction, being treated or prevented. Also, for purposes herein, “prevention” can encompass delaying the onset of the disease, or a symptom or condition thereof.

Cardiac Metabolic Modifiers

As used herein, the term “cardiac metabolic modifier” refers to a compound which modulates a myocardial metabolic pathway to influence (e.g., improve, maintain) cardiac efficiency. In some embodiments, the cardiac metabolic modifier directly modulates a myocardial metabolic pathway to influence (e.g., improve, maintain) cardiac efficiency. In alternative embodiments, the cardiac metabolic modifier indirectly modulates a myocardial metabolic pathway to influence (e.g., improve, maintain) cardiac efficiency. As used herein, the term “cardiac efficiency” refers to the ratio between the work produced (blood pumped) and input of energy in order to make the heart work. More specifically, cardiac efficiency is the ratio of stroke work to oxygen consumption. Methods of measuring cardiac efficiency are known in the art, and include invasive procedures, e.g., cardiac catheterization, and non-invasive procedures, e.g., positron emission topograph (PET). See, for example, Steendijk et al., Heart Metab 39: 33-36 (2008), Knaapen et al., Heart Metab. 39:14-19 (2008). In some aspects, the cardiac metabolic modifier influences cardiac efficiency, such that a cardiac efficiency of about 20% to about 25% (e.g., 20%, 21%, 22%, 23%, 24%, 25%) is attained or maintained.

In some embodiments, the cardiac metabolic modifier modulates one or more of: fatty acid oxidation in the myocardium, carbohydrate oxidation in the myocardium, glycolysis in the myocardium, myofilament calcium sensitivity, ion channel activity in the myocardium, or sensitivity to insulin in the myocardium.

In some embodiments, the cardiac metabolic modifier inhibits fatty acid oxidation. Methods of assaying fatty acid oxidation are known in the art. See, for example, Wang et al., J Pharmacology and Experimental Therapeutics 321: 213-220 (2007).

In some embodiments, the cardiac metabolic modifier alters carbohydrate oxidation. Methods of assaying carbohydrate oxidation are known in the art. See, for example, Wang et al., J Pharmacology and Experimental Therapeutics 321: 213-220 (2007).

Alternatively or additionally, in some embodiments, the cardiac metabolic modifier lowers myofilament calcium sensitivity. In exemplary aspects, the cardiac metabolic modifier lowers calcium sensitivity of myofilaments of cardiac muscle. Methods of assaying myofilament calcium sensitivity are known in the art and in the EXAMPLES section set forth below. In exemplary aspects, the cardiac metabolic modifier binds to myofilaments of cardiac muscle.

Alternatively or additionally, in some embodiments, the cardiac metabolic modifier inhibits an ion channel, such as a sodium ion channel, a calcium ion channel, a potassium channel. Methods of assaying inhibition of ion channels are known in the art and include, for example, Borgland et al., J Physiology 536: 35-47 (2001).

In some embodiments, the cardiac metabolic modifier comprises a structure of Formula I:

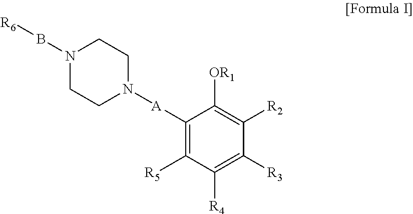

wherein A comprises a main chain of 1-8 atoms, each atom of which is independently C, O, N, or S, and each atom or which is optionally bound to an additional group selected from C1-C8 alkyl, C1-C8 alkoxy, OH, NH₂, NH(C1-C4 alkyl) and SH;

wherein R₁ is H or a C1-C8 alkyl;

wherein each of R₂, R₃, R₄, and R₅ independently is H, a C1-C8 alkyl, or a C1-C8 alkoxy;

wherein B is H or comprises a main chain of 1-8 atoms, each atom of which is independently C, O, N, or S, and each atom of which is optionally bound to an additional group; and,

wherein R₆ is absent or phenyl, which phenyl is optionally substituted with 1 to 5 groups, each group of which is independently C1-C8 alkyl, C1-C8 alkoxy, or OH.

As used herein, “alkyl” refers to straight chained and branched saturated hydrocarbon groups, nonlimiting examples of which include methyl, ethyl, and straight and branched propyl, butyl, pentyl, hexyl, heptyl, and octyl groups containing the indicated number of carbon atoms. The term Cn means the alkyl group has “n” carbon atoms. For example, C1-C7 alkyl refers to alkyl groups having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms). Accordingly, the C1-C8 alkyl can be a methyl, ethyl, propyl, butyl, C5 alkyl, C6 alkyl, C7 alkyl, or C8 alkyl, of which the propyl, butyl, C5 alkyl, C6 alkyl, C7 alkyl, or C8 alkyl is a straight chain alkyl or branched alkyl.

As used herein “alkoxy” refers to —OR, wherein R is alkyl (e.g., a straight or branched chain alkyl group). Examples of alkoxy groups include, but are not limited to, methoxy, ethoxy, propoxy, isopropoxy, butoxy, n-butoxy, sec-butoxy, t-butoxy and the like. Accordingly, the C1-C8 alkoxy can be methoxy, ethoxy, C3 alkoxy, C4 alkoxy, C5 alkoxy, C6 alkoxy, C7 alkoxy, or C8 alkoxy, or which the C3 alkoxy, C4 alkoxy, C5 alkoxy, C6 alkoxy, C7 alkoxy, or C8 alkoxy is a straight chain alkoxy or branched alkoxy.

As used herein “NH(C1-C4 alkyl)” refers to nitrogen bound to both H and a C1-C4 alkyl.

With regard to Formula I, A comprises a main chain of 1-8 atoms, each atom of which is independently C, O, N, or S. In some aspects, A comprises a main chain of a single atom selected from C, O, N, and S. In some aspects, A comprises a main chain of 2-8 atoms (e.g., 2, 3, 4, 5, 6, 7, or 8 atoms), each atom of which is independently C, O, N, or S.

Each atom of the main chain of A is optionally bound to an additional group. In some aspects, the additional group is selected from C1-C8 alkyl, C1-C8 alkoxy, OH, NH2, NH(C1-C4 alkyl) and SH. In some aspects, every atom of the main chain is bound to an additional group. In other aspects, one or more, but not all, atoms of the main chain are bound to an additional group. In some instances, one atom of the main chain is bound to an additional group. In some instances, 2, 3, 4, 5, 6, or 7 atoms of the main chain is bound to an additional group.

In some aspects, A is (CH₂)₁₋₈. In some aspects, A is (CH₂)₁₋₆. In some aspects, A is (CH₂)₁₋₄. In some aspects, A is (CH₂)₁ or (CH₂)₂. In some aspects, A is (CH₂)₁.

In other aspects, A comprises a structure of Formula IV:

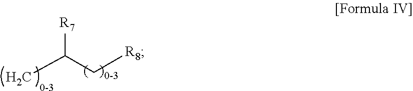

wherein R₇ is OH, C1-C4 alkyl, C1-C4 alkoxy, NH2, or NH(C1-C4 alkyl); and wherein R₈ is O or NH. In some aspects, when A comprises a structure of Formula IV, R₇ is OH and R₈ is O or NH. In some aspects, when A comprises a structure of Formula IV, R₇ is OH, C1-C4 alkyl, C1-C4 alkoxy, NH2, or NH(C1-C4 alkyl) and R₈ is O. In particular aspects, when A comprises a structure of Formula IV, R₇ is OH and R₈ is O. In some aspects, A comprises

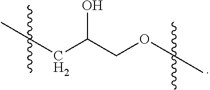

With regard to Formula I, R₁ is H or a C1-C8 alkyl. In particular aspects, R₁ is CH₃.

With regard to Formula I, each of R₂, R₃, R₄, and R₅ independently is H, a C1-C8 alkyl, or a C1-C8 alkoxy. In some aspects, each of R₂, R₃, R₄, and R₅ independently is H or methoxy. In alternative embodiments, each of R₂ and R₃ is a methoxy, and each of R₄ and R₅ is H.

With regard to Formula I, B is H or comprises a main chain of 1-8 atoms, each atom of which is independently C, O, N, or S. In some aspects, B comprises a main chain of a single atom selected from C, O, N, and S, while in other aspects, B comprises a main chain of 2-8 atoms (e.g., 2, 3, 4, 5, 6, 7, or 8 atoms), each atom of which is independently selected from C, O, N, and S.

Each atom of the main chain of B is optionally bound to an additional group. In some aspects, the additional group is selected from C1-C8 alkyl, C1-C8 alkoxy, OH, NH2, NH(C1-C4 alkyl) and SH. In some aspects, every atom of the main chain is bound to an additional group. In other aspects, one or more, but not all, atoms of the main chain are bound to an additional group. In some instances, one atom of the main chain is bound to an additional group. In some instances, 2, 3, 4, 5, 6, or 7 atoms of the main chain is bound to an additional group.

In some embodiments, B comprises H and R₆ is absent. In alternative embodiments, B comprises a structure of Formula V:

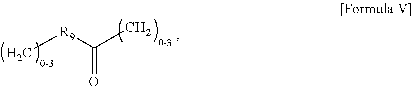

wherein R₉ is NH or O.

In some embodiments, when B comprises a structure of Formula V, R₉ is NH. In further aspects, B comprises a structure of

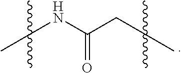

With regard to Formula I, R₆ is absent or phenyl, which phenyl is optionally substituted with 1 to 5 (e.g., 1, 2, 3, 4, 5) groups, each group of which is independently selected from C1-C8 alkyl, C1-C8 alkoxy, and OH. In some aspects, R₆ is absent. In alternative aspects, when R₆ is present and comprises phenyl substituted with 1 to 5 (e.g., 1, 2, 3, 4, 5) methyl groups. In specific aspects, R₆ comprises phenyl substituted with two methyl groups, one at each of the ortho positions.

In some aspects, the cardiac metabolic modifier comprises a compound of Formula II, or a pharmaceutically acceptable salt thereof or a conjugate thereof:

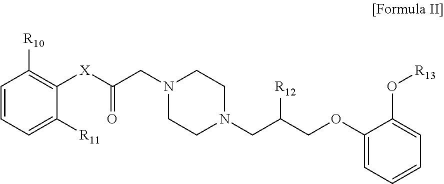

wherein each of R₁₀, R₁₁, and R₁₃ independently is C1-C3 alkyl,

wherein X is NH or O; and

wherein R₁₂ is OH or C1-C3 alkyl.

In some embodiments, the compound of Formula II comprises the following structure:

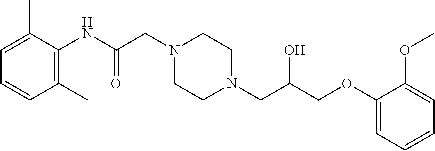

In some aspects, the compound of Formula II is ranolazine, or a pharmaceutically acceptable salt or a conjugate of ranolazine.

In some aspects, the cardiac metabolic modifier is a compound of Formula III, or a pharmaceutically acceptable salt thereof or a conjugate thereof:

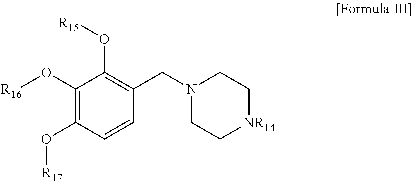

wherein each of R₁₄, R₁₅, R₁₆, and R₁₇ independently is H or C1-C3 alkyl.

In some embodiments, the compound of Formula II comprises the following structure:

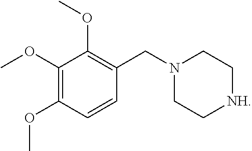

In some aspects, the compound of Formula III is trimetazidine, or a pharmaceutically acceptable salt or a conjugate of trimetazidine.

Pharmaceutically Acceptable Salts

In some embodiments, the cardiac metabolic modifiers are in the form of a salt, e.g., a pharmaceutically acceptable salt. Such salts can be prepared in situ during the final isolation and purification of the cardiac metabolic modifier or separately prepared by reacting a free base function with a suitable acid. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate.

Basic addition salts also can be prepared in situ during the final isolation and purification of the cardiac metabolic modifier, or by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like.

Further, basic nitrogen-containing groups can be quaternized with such cardiac metabolic modifiers as lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

Conjugates

In some embodiments, the cardiac metabolic modifier is in the form of a conjugate, e.g., is conjugated to a heterologous moiety. As used herein, the term “heterologous moiety” is synonymous with the term “conjugate moiety” and refers to any molecule (chemical or biochemical, naturally-occurring or non-coded) which is different from the cardiac metabolic modifiers described herein. Exemplary conjugate moieties that can be linked to any of the cardiac metabolic modifiers described herein include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments a conjugate is provided comprising a cardiac metabolic modifier and a plasma protein, wherein the plasma protein is selected from the group consisting of albumin, transferin, fibrinogen and globulins. In some embodiments the plasma protein moiety of the conjugate is albumin or transferin. The conjugate in some embodiments comprises one or more of the cardiac metabolic modifiers described herein and one or more of: a peptide, a polypeptide, a nucleic acid molecule, an antibody or fragment thereof, a polymer, a quantum dot, a small molecule (which is distinct from the cardiac metabolic modifiers described herein), a toxin, a diagnostic agent, a carbohydrate, an amino acid.

In some embodiments, the heterologous moiety is a polymer. In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

In some aspects, the polymer is a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamines and hydrophobic proteins)), as well as any copolymer or mixture thereof. In general, these materials degrade either by enzymatic hydrolysis or exposure to water in vivo, by surface or bulk erosion.

In some aspects, the polymer is a bioadhesive polymer, such as a bioerodible hydrogel described by H. S. Sawhney, C. P. Pathak and J. A. Hubbell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In some embodiments, the polymer is a water-soluble polymer or a hydrophilic polymer. Hydrophilic polymers are further described herein under “Hydrophilic Moieties.” Suitable water-soluble polymers are known in the art and include, for example, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof.

In specific embodiments, the polymer is a polyalkylene glycol, including, for example, polyethylene glycol (PEG).

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is attached via non-covalent or covalent bonding to the cardiac metabolic modifier of the present disclosure. In certain aspects, the heterologous moiety is attached to the cardiac metabolic modifier of the present disclosure via a linker. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

The cardiac metabolic modifier in some embodiments is linked to conjugate moieties via direct covalent linkage. In some embodiments, reactive groups on the cardiac metabolic modifier or conjugate moiety include, e.g., an aldehyde, amino, ester, thiol, α-haloacetyl, maleimido or hydrazino group. Derivatizing agents include, for example, maleimidobenzoyl sulfosuccinimide ester, N-hydroxysuccinimide (through lysine residues), glutaraldehyde, succinic anhydride or other agents known in the art. Alternatively, the conjugate moieties can be linked to the cardiac metabolic modifier indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Carboxyl groups are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Another type of covalent modification involves chemically or enzymatically coupling glycosides to the cardiac metabolic modifier. Sugar(s) may be attached to free carboxyl groups, free sulfhydryl groups, free hydroxyl groups or an amide group. These methods are described in WO87/05330 published 11 Sep. 1987, and in Aplin and Wriston, CRC Crit. Rev. Biochem., pp. 259-306 (1981).

In some embodiments, the conjugate comprises a linker that joins the cardiac metabolic modifier to the heterologous moiety. In some aspects, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. In some embodiments, the linker is an amino acid or a peptidyl linker. Such peptidyl linkers may be any length. Exemplary linkers are from about 1 to 50 amino acids in length, 5 to 50, 3 to 5, 5 to 10, 5 to 15, or 10 to 30 amino acids in length.

Conjugates: Hydrophilic Moieties

The cardiac metabolic modifiers described herein can be further modified to improve its solubility and stability in aqueous solutions at physiological pH, while retaining its biological activity. Hydrophilic moieties such as PEG groups can be attached to the cardiac metabolic modifiers under any suitable conditions known in the art, including, for example, via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the cardiac metabolic modifier by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1,3-dioxolane, poly-1,3,6-trioxane, ethylene/maleic anhydride copolymer, poly β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by □1-6 linkages. Dextran is available in many molecular weight ranges, e.g., about 1 kD to about 100 kD, or from about 5, 10, 15 or 20 kD to about 20, 30, 40, 50, 60, 70, 80 or 90 kD. Linear or branched polymers are contemplated. Resulting preparations of conjugates may be essentially monodisperse or polydisperse, and may have about 0.5, 0.7, 1, 1.2, 1.5 or 2 polymer moieties per compound.

Conjugates: Multimers

In some embodiments, the conjugate comprising the cardiac metabolic modifier is in the form of a multimer or dimer, including homo- or hetero-multimers or homo- or hetero-dimers. Two or more of the cardiac metabolic modifiers can be linked together using standard linking agents and procedures known to those skilled in the art. In certain embodiments, the linker connecting the two (or more) analogs is PEG, e.g., a 5 kDa PEG, 20 kDa PEG. In some embodiments, the linker is a disulfide bond. For example, each monomer of the dimer may comprise a sulfhydryl and the sulfur atom of each participates in the formation of the disulfide bond.

Conjugates: Targeted Forms

One of ordinary skill in the art will readily appreciate that the cardiac metabolic modifiers of the disclosure can be modified in any number of ways, such that the therapeutic or prophylactic efficacy of the cardiac metabolic modifier of the present disclosures is increased through the modification. For instance, the cardiac metabolic modifier of the present disclosure can be conjugated either directly or indirectly through a linker to a targeting moiety. The practice of conjugating compounds to targeting moieties is known in the art. See, for instance, Wadhwa et al., J Drug Targeting, 3, 111-127 (1995) and U.S. Pat. No. 5,087,616. The term “targeting moiety” as used herein, refers to any molecule or agent that specifically recognizes and binds to a cell-surface receptor, such that the targeting moiety directs the delivery of the cardiac metabolic modifier of the present disclosures to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups. The term “linker” in some embodiments refers to any agent or molecule that bridges the cardiac metabolic modifier of the present disclosures to the targeting moiety. One of ordinary skill in the art recognizes that sites on the cardiac metabolic modifier of the present disclosures, which are not necessary for the function of the cardiac metabolic modifier, are ideal sites for attaching a linker and/or a targeting moiety, provided that the linker and/or targeting moiety, once attached to the cardiac metabolic modifier, do(es) not interfere with the function of the cardiac metabolic modifier, i.e., the ability to treat diastolic dysfunction, as described herien.

Pharmaceutical Compositions and Formulations

In some embodiments, the cardiac metabolic modifier of the present disclosures, the pharmaceutically acceptable salt thereof, or the conjugate comprising the cardiac metabolic modifier, is formulated into a pharmaceutical composition comprising the cardiac metabolic modifier, the pharmaceutically acceptable salt thereof, or the conjugate comprising the cardiac metabolic modifier, along with a pharmaceutically acceptable carrier, diluent, or excipient.

In some embodiments, the cardiac metabolic modifier is present in the pharmaceutical composition at a purity level suitable for administration to a patient. In some embodiments, the cardiac metabolic modifier has a purity level of at least about 90%, about 91%, about 92%, about 93%, about 94%, about 95%, about 96%, about 97%, about 98% or about 99%, and a pharmaceutically acceptable diluent, carrier or excipient. The pharmaceutical composition in some aspects comprises the cardiac metabolic modifier of the present disclosure at a concentration of at least A, wherein A is about 0.001 mg/ml, about 0.01 mg/ml, 0 about 1 mg/ml, about 0.5 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml or higher. In some embodiments, the pharmaceutical composition comprises the cardiac metabolic modifier at a concentration of at most B, wherein B is about 30 mg/ml, about 25 mg/ml, about 24 mg/ml, about 23, mg/ml, about 22 mg/ml, about 21 mg/ml, about 20 mg/ml, about 19 mg/ml, about 18 mg/ml, about 17 mg/ml, about 16 mg/ml, about 15 mg/ml, about 14 mg/ml, about 13 mg/ml, about 12 mg/ml, about 11 mg/ml, about 10 mg/ml, about 9 mg/ml, about 8 mg/ml, about 7 mg/ml, about 6 mg/ml, about 5 mg/ml, about 4 mg/ml, about 3 mg/ml, about 2 mg/ml, about 1 mg/ml, or about 0.1 mg/ml. In some embodiments, the compositions may contain an cardiac metabolic modifier at a concentration range of A to B mg/ml, for example, about 0.001 to about 30.0 mg/ml.

Depending on the route of administration, the particular cardiac metabolic modifier intended for use, as well as other factors, the pharmaceutical composition may comprise additional pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

Accordingly, in some embodiments, the pharmaceutical composition comprises any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, α-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, f3-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC)chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil, type II, hydroxyethyl cellulose, 2-hydroxyethyl-β-cyclodextrin, hydroxypropyl cellulose, low-substituted hydroxypropyl cellulose, 2-hydroxypropyl-β-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, postassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-β-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety, discloses various components used in formulating pharmaceutically acceptable compositions and known techniques for the preparation thereof. Except insofar as any conventional agent is incompatible with the pharmaceutical compositions, its use in pharmaceutical compositions is contemplated. Supplementary active ingredients also can be incorporated into the compositions.

In some embodiments, the foregoing component(s) may be present in the pharmaceutical composition at any concentration, such as, for example, at least A, wherein A is 0.0001% w/v, 0.001% w/v, 0.01% w/v, 0.1% w/v, 1% w/v, 2% w/v, 5% w/v, 10% w/v, 20% w/v, 30% w/v, 40% w/v, 50% w/v, 60% w/v, 70% w/v, 80% w/v, or 90% w/v. In some embodiments, the foregoing component(s) may be present in the pharmaceutical composition at any concentration, such as, for example, at most B, wherein B is 90% w/v, 80% w/v, 70% w/v, 60% w/v, 50% w/v, 40% w/v, 30% w/v, 20% w/v, 10% w/v, 5% w/v, 2% w/v, 1% w/v, 0.1% w/v, 0.001% w/v, or 0.0001%. In other embodiments, the foregoing component(s) may be present in the pharmaceutical composition at any concentration range, such as, for example from about A to about B. In some embodiments, A is 0.0001% and B is 90%.

The pharmaceutical compositions may be formulated to achieve a physiologically compatible pH. In some embodiments, the pH of the pharmaceutical composition may be at least 5, at least 5.5, at least 6, at least 6.5, at least 7, at least 7.5, at least 8, at least 8.5, at least 9, at least 9.5, at least 10, or at least 10.5 up to and including pH 11, depending on the formulation and route of administration. In certain embodiments, the pharmaceutical compositions may comprise buffering agents to achieve a physiological compatible pH. The buffering agents may include any compounds capabale of buffering at the desired pH such as, for example, phosphate buffers (e.g., PBS), triethanolamine, Tris, bicine, TAPS, tricine, HEPES, TES, MOPS, PIPES, cacodylate, MES, and others. In certain embodiments, the strength of the buffer is at least 0.5 mM, at least 1 mM, at least 5 mM, at least 10 mM, at least 20 mM, at least 30 mM, at least 40 mM, at least 50 mM, at least 60 mM, at least 70 mM, at least 80 mM, at least 90 mM, at least 100 mM, at least 120 mM, at least 150 mM, or at least 200 mM. In some embodiments, the strength of the buffer is no more than 300 mM (e.g., at most 200 mM, at most 100 mM, at most 90 mM, at most 80 mM, at most 70 mM, at most 60 mM, at most 50 mM, at most 40 mM, at most 30 mM, at most 20 mM, at most 10 mM, at most 5 mM, at most 1 mM).

Routes of Administration

With regard to the invention, the cardiac metabolic modifier, pharmaceutical composition comprising the same, conjugate comprising the same, or pharmaceutically acceptable salt thereof, may be administered to the subject by any suitable route of administration. The following discussion on routes of administration is merely provided to illustrate exemplary embodiments and should not be construed as limiting the scope in any way.

Formulations suitable for oral administration can consist of (a) liquid solutions, such as an effective amount of the cardiac metabolic modifier of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the cardiac metabolic modifier of the present disclosure in a flavor, usually sucrose and acacia or tragacanth, as well as pastilles comprising the cardiac metabolic modifier of the present disclosure in an inert base, such as gelatin and glycerin, or sucrose and acacia, emulsions, gels, and the like containing, in addition to, such excipients as are known in the art.

The cardiac metabolic modifiers of the present disclosure, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the cardiac metabolic modifier is formulated into a powder blend or into microparticles or nanoparticles. Suitable pulmonary formulations are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The cardiac metabolic modifier of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the cardiac metabolic modifier of the present disclosure in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the cardiac metabolic modifier of the present disclosures can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the cardiac metabolic modifier of the disclosure can be formulated as inclusion complexes, such as cyclodextrin inclusion complexes, or liposomes.

In exemplary aspects, the cardiac metabolic modifier is administered to the subject by more than one route of administration. In exemplary aspects, in the methods provided herein, the cardiac metabolic modifier is intravenously administered to the subject and orally administered to the subject. The intravenous administration may occur at the same time of oral administration. Alternatively, the intravenous administration may occur before and/or after the oral administration. In exemplary aspects, the method comprises multiple intravenous administrations an/or oral administrations of the cardiac metabolic modifier, and the administrations occur in a particular order and at specific dosages.

Dosages

The cardiac metabolic modifiers of the disclosure are believed to be useful in methods of treating a diastolic dysfunction, as well as related conditions, as described herein. For purposes of the disclosure, the amount or dose of the cardiac metabolic modifier administered should be sufficient to effect, e.g., a therapeutic or prophylactic response, in the subject or animal over a reasonable time frame. For example, the dose of the cardiac metabolic modifier of the present disclosure should be sufficient to treat diastolic dysfunction as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular cardiac metabolic modifier and the condition of the animal (e.g., human), as well as the body weight of the animal (e.g., human) to be treated.

Many assays for determining an administered dose are known in the art. For purposes herein, an assay, which comprises comparing the extent to which diastolic dysfunction is treated upon administration of a given dose of the cardiac metabolic modifier of the present disclosure to a mammal among a set of mammals, each set of which is given a different dose of the cardiac metabolic modifier, could be used to determine a starting dose to be administered to a mammal. The extent to which diastolic dysfunction is treated upon administration of a certain dose can be assayed by methods known in the art, including, for instance, the methods described in the Examples set forth below.

The dose of the cardiac metabolic modifier of the present disclosure also will be determined by the existence, nature and extent of any adverse side effects that might accompany the administration of a particular cardiac metabolic modifier of the present disclosure. Typically, the attending physician will decide the dosage of the cardiac metabolic modifier of the present disclosure with which to treat each individual patient, taking into consideration a variety of factors, such as age, body weight, general health, diet, sex, cardiac metabolic modifier of the present disclosure to be administered, route of administration, and the severity of the condition being treated. By way of example and not intending to limit the invention, the dose of the cardiac metabolic modifier of the present disclosure can be about 0.0001 to about 1 g/kg body weight of the subject being treated/day, from about 0.0001 to about 0.001 g/kg body weight/day, or about 0.01 mg to about 1 g/kg body weight/day.

In some embodiments, the cardiac metabolic modifier is formulated for injection, is a compound of Formula II, e.g., ranolazine, and is administered to the subject at a dose between about 1 and about 20 mg/kg body weight of the subject for an injection, (e.g., between about 5 and about 15 mg/kg, between about 10 to about 12 mg/kg, about 9 mg/kg, about 10 mg/kg, about 11 mg/kg, about 12 mg/kg). In some embodiments, the cardiac metabolic modifier is formulated for infusion (e.g., intravenous infusion), is a compound of Formula II, e.g., ranolazine, and is administered at a dose between about 1 mg/kg/h to about 20 mg/kg/h (e.g., about 1 mg/kg/h, about 2 mg/kg/h, about 3 mg/kg/h, about 4 mg/kg/h, about 5 mg/kg/h, about 6 mg/kg/h, about 7 mg/kg/h, about 8 mg/kg/h, about 9 mg/kg/h, about 10 mg/kg/h, about 11 mg/kg/h, about 12 mg/kg/h, about 13 mg/kg/h, about 14 mg/kg/h, about 15 mg/kg/h, about 16 mg/kg/h, about 17 mg/kg/h, about 18 mg/kg/h, about 19 mg/kg/h, about 20 mg/kg/h)

In some embodiments, wherein the cardiac metabolic modifier is formulated for oral administration and is a compound of Formula II, e.g., ranolazine, the dose administered to the subject is between about 100 and about 2000 mg (e.g., about 100 mg, about 200 mg, about 250 mg, about 300 mg, about 400 mg, about 500 mg, about 600 mg, about 700 mg, about 750 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg). In some aspects, the oral dosage is administered once daily, twice daily, three times daily, or four times daily.

In some embodiments, the dosage of the cardiac metabolic modifier is any of the above dosages, but the cardiac metabolic modifier is other than a compound of Formula II (e.g., ranolazine).

In some embodiments, the administered dose of the cardiac metabolic modifier (e.g., any of the doses described above), provides the subject with a plasma concentration of the cardiac metabolic modifier of at least or about 500 nM. In some aspects, the administered dose of the cardiac metabolic modifier provides the subject with a plasma concentration of the cardiac metabolic modifier within a range of about 500 nM to about 2500 nM (e.g., about 750 nM to about 2000 nM, about 1000 nM to about 1500 nM). In some aspects, the dose of the cardiac metabolic modifier provides the subject with a plasma concentration of the cardiac metabolic modifier which is below 100 μmol/L, e.g., below 50 μmol/L, below 25 μmol/L, below 10 μmol/L.

In exemplary aspects, the methods provided herein comprise administration of the cardiac metabolic modifier at more than one dose. In some aspects, (i) the first dose is within a range of about 150 mg/h to about 250 mg/h (e.g., about 175 mg/h to about 225 mg/h, about 200 mg/h to about 215 mg/h) (ii) the second dose is within a range of about 30 mg/h to about 100 mg/h (e.g., about 35 mg/h to about 95 mg/h, about 40 mg/h to about 90 mg/h, about 45 mg/h to about 85 mg/h, about 50 mg/h to about 80 mg/h, about 55 mg/h to about 75 mg/h, about 60 to about 70 mg/h) (iii) each administration of the third dose is within a range of about 350 mg to about 1500 mg (e.g., about 400 mg to about 1200, about 500 mg to about 1000 mg, about 600 mg to about 800 mg), or (iv) a combination thereof.

In exemplary aspects, the methods provided herein comprise administration of the cardiac metabolic modifier at more than one dose, and the doses depend on whether or not the subject is taking another therapeutic agent. For example, if the subject is taking a CYP3A inhibitor, then the dose(s) of the cardiac metabolic modifier may be decreased, as compared to the dose(s) of the cardiac metabolic modifier administered to a subject not taking a CYP3A inhibitor. For example, (i) the first dose is within a range of about 180 mg/h to about 220 mg/h (e.g., about 185 mg/h to about 215 mg/h, about 180 mg/h to about 210 mg/h, about 185 to about 205 mg/h, about 190 mg/h to about 200 mg/h), (ii) the second dose is within a range of about 30 mg/h to about 50 mg/h (e.g., about 30 mg/h, about 35 mg/h, about 40 mg/h, about 45 mg/h, about 50 mg/h), if the subject is taking a CYP3A inhibitor, or is within a range of about 60 mg to about 100 mg (e.g., about 60 mg, about 70 mg, about 80 mg, about 90 mg, about 100 mg), if the subject is not taking a CYP3A inhibitor, (iii) each administration of the third dose is within a range of about 350 mg to about 650 mg (e.g., about 400 mg to about 600 mg, about 450 mg to about 550 mg), if the subject is taking a CYP3A inhibitor, or is within a range of about 900 mg to about 1100 mg (e.g., about 950 to about 1050 mg, about 1000 to about 1100 mg), if the subject is not taking a CYP3A inhibitor, or (iv) a combination thereof. In exemplary aspects, the CYP3A inhibitor is verapamil, diltiazem, or erythromycin

Controlled Release Formulations

In some embodiments, the cardiac metabolic modifiers described herein can be modified into a depot form, such that the manner in which the cardiac metabolic modifier of the present disclosures is released into the body to which it is administered is controlled with respect to time and location within the body (see, for example, U.S. Pat. No. 4,450,150). Depot forms of cardiac metabolic modifiers of the present disclosures can be, for example, an implantable composition comprising the cardiac metabolic modifiers and a porous or non-porous material, such as a polymer, wherein the cardiac metabolic modifiers is encapsulated by or diffused throughout the material and/or degradation of the non-porous material. The depot is then implanted into the desired location within the body of the subject and the cardiac metabolic modifiers is released from the implant at a predetermined rate.

The pharmaceutical composition comprising the cardiac metabolic modifier in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374: 46-52 (2009) and International Patent Application Publication Nos. WO 2008/130158, WO2004/033036; WO2000/032218; and WO 1999/040942. In exemplary aspects, the method provided herein comprises administration of one or more immediate release dosages of the cardiac metabolic modifier and one or more sustained or extended release dosages of the cardiac metabolic modifier.

The instant compositions may further comprise, for example, micelles or liposomes, or some other encapsulated form, or may be administered in an extended release form to provide a prolonged storage and/or delivery effect. The disclosed pharmaceutical formulations may be administered according to any regime including, for example, daily (1 time per day, 2 times per day, 3 times per day, 4 times per day, 5 times per day, 6 times per day), every two days, every three days, every four days, every five days, every six days, weekly, bi-weekly, every three weeks, monthly, or bi-monthly.

Combinations

In some embodiments, the cardiac metabolic modifiers described herein are administered alone, and in alternative embodiments, the cardiac metabolic modifiers described herein are administered in combination with another therapeutic agent which aims to treat or prevent any of the diseases or medical conditions described herein, e.g., diastolic dysfunction. In exemplary embodiments, a cardiac metabolic modifier of a first structure is co-administered with (simultaneously or sequentially) another cardiac metabolic modifier of different structure. In alternative or additional embodiments, the cardiac metabolic modifiers described herein may be co-administered with (simultaneously or sequentially) a therapeutic agent for the treatment of hypertension, including, for example, a thiazide diuretic (e.g., chlorothiazine, hydrochlorothiazide, metolazone), a beta blocker (a.k.a, beta-adrenergic blocking agent (e.g., acebutolol, atenolol, bisoprolol, carvedilol, metoprolol, nadolol, nebivolol, penbutolol, propranolol)), an angiotensin-convertine enzyme (ACE) inhibitor (e.g., benazepril, captopril, enalapril, fosinopril, lisinopril, moexipril, perindopril, quinapril, ramipril, trandolarpil), an angiotensin II receptor blocker (a.k.a., ARBs (e.g., candesartan, eprosartan, irbesartan, losartan, olmesartan, telmisartan, valsartan)), a calcium channel blocker (a.k.a., calcium antagonist, (e.g., amlodipine, diltiazem, felodipine, isradipine, nicardipine, nifedipine, nisoldipine, verapramil)), a rennin inhibitor (e.g., Aliskiren), an alpha blocker (a.k.a, an alpha-adrenergic antagonist, alpha-adrenergic blocking agent, adrenergic blocking agent, alpha-blocking agent, (e.g., doxazosin, prazosin, terazosin, tamsulosin, alfuzosin)), an alpha-beta blocker (a.k.a, alpha-beta adrenergic blocker (e.g., carvedilol, labetalol), a central-acting agent (a.k.a., central adrenergic inhibitor, central alpha agonist, central agonist, (e.g., clonidine, guanfacine, methyldopa)), a vasodilator (e.g., hydralazine, minoxidil).

In alternative or additional embodiments, the cardiac metabolic modifier is co-administered with (simultaneously or sequentially) a therapeutic agent for the treatment of diabetes or obesity. Anti-diabetic agents known in the art or under investigation include insulin, leptin, Peptide YY (PYY), Pancreatic Peptide (PP), fibroblast growth factor 21 (FGF21), Y2Y4 receptor agonists, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARy inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; glucokinase activators (GKA); glucagon receptor antagonists (GRA); or FBPase (fructose 1,6-bisphosphatase) inhibitors, GLP-1 agonists.

Anti-obesity agents known in the art or under investigation include appetite suppressants, including phenethylamine type stimulants, phentermine (optionally with fenfluramine or dexfenfluramine), diethylpropion (Tenuate®), phendimetrazine (Prelu-2®, Bontril®), benzphetamine (Didrex®), sibutramine (Meridia®, Reductil®); rimonabant (Acomplia®), other cannabinoid receptor antagonists; oxyntomodulin; fluoxetine hydrochloride (Prozac); Qnexa (topiramate and phentermine), Excalia (bupropion and zonisamide) or Contrave (bupropion and naltrexone); or lipase inhibitors, similar to XENICAL (Orlistat) or Cetilistat (also known as ATL-962), or GT 389-255.

In some embodiments, the cardiac metabolic modifier is administered in combination with aspirin, or other therapeutic agent which promotes cardiac efficiency. In some aspects, the cardiac metabolic modifier is administered in combination with tetahydrobiopterin (BH₄) or an analog thereof.

In view of the foregoing, the invention further provides pharmaceutical compositions and kits additionally comprising one of these other therapeutic agents in combination with the cardiac metabolic modifier. The additional therapeutic agent may be administered simultaneously or sequentially with the cardiac metabolic modifier of the present disclosure. In some aspects, the cardiac metabolic modifier is administered before the additional therapeutic agent, while in other aspects, the cardiac metabolic modifier is administered after the additional therapeutic agent.

Subjects

In some embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human.

In some embodiments, the subject is in need of treatment for diastolic dysfunction, e.g., any of the diastolic dysfunctions described herein (e.g., diastolic dysfunction with preserved ejection fraction, diastolic dysfunction with preserved left ventricular function, diastolic dysfunction with preserved systolic function or diastolic dysfunction without systolic dysfunction, diastolic dysfunction characterized by (i) a lack of increased late I_{Na} in cardiomyocytes, (ii) an increase in myofilament calcium sensitivity, or (iii) a combination thereof). In exemplary embodiments, the subject exhibits an ejection fraction which is greater than or about 45%, e.g., greater than or about 50%.

In some embodiments, the subject suffers from a form of diastolic dysfunction epidemiologically associated with hypertension, also known as, high blood pressure. In this regard, the subject in some embodiments, suffers from hypertension as well as diastolic dysfunction with preserved ejection fraction. Hypertension is a chronic medical condition in which the systemic arterial blood pressure is elevated. The hypertension in some embodiments is classified as a primary hypertension for which no medical cause is found. In some embodiments, the hypertension is a secondary hypertension caused by another condition that affects the kidneys, arteries, heart, or endocrine system.

A systolic or the diastolic blood pressure measurement higher than the accepted normal values for the age of the individual is classified as prehypertension or hypertension.

| | Systolic pressure | | Diastolic pressure | |
|---|---|---|---|---|
| Classification | mmHg | kPa | mmHg | kPa |
| Normal | 90-119 | 12-15.9 | 60-79 | 8.0-10.5 |
| Prehypertension | 120-139 | 16.0-18.5 | 80-89 | 10.7-11.9 |
| Stage 1 | 140-159 | 18.7-21.2 | 90-99 | 12.0-13.2 |
| Stage 2 | ≧160 | ≧21.3 | ≧100 | ≧13.3 |
| Isolated systolic | ≧140 | ≧18.7 | <90 | <12.0 |
| hypertension | | | | |

Source: Chobanian et al. (2003)

Hypertension has several sub-classifications including, hypertension stage I, hypertension stage II, and isolated systolic hypertension. Isolated systolic hypertension refers to elevated systolic pressure with normal diastolic pressure and is common in the elderly. These classifications are made after averaging a patient's resting blood pressure readings taken on two or more office visits. Individuals older than 50 years are classified as having hypertension if their blood pressure is consistently at least 140 mmHg systolic or 90 mmHg diastolic. Patients with blood pressures higher than 130/80 mmHg with concomitant presence of diabetes mellitus or kidney disease require further treatment (Chobanian et al. (December 2003). “Seventh report of the Joint National Committee on Prevention, Detection, Evaluation, and Treatment of High Blood Pressure”. Hypertension 42(6): 1206-52.)

In some aspects, the subject suffers from diastolic dysfunction which is epidemiologically associated with a metabolic disease or metabolic syndrome. In this regard, the subject in some embodiments suffers from diastolic dysfunction and a metabolic disease or metabolic syndrome. Metabolic Syndrome, also known as metabolic syndrome X, insulin resistance syndrome or Reaven's syndrome, is a disorder that affects over 50 million Americans. Metabolic Syndrome is typically characterized by a clustering of at least three or more of the following risk factors: (1) abdominal obesity (excessive fat tissue in and around the abdomen), (2) atherogenic dyslipidemia (blood fat disorders including high triglycerides, low HDL cholesterol and high LDL cholesterol that enhance the accumulation of plaque in the artery walls), (3) elevated blood pressure, (4) insulin resistance or glucose intolerance, (5) prothrombotic state (e.g., high fibrinogen or plasminogen activator inhibitor-1 in blood), and (6) pro-inflammatory state (e.g., elevated C-reactive protein in blood). Other risk factors may include aging, hormonal imbalance and genetic predisposition.

Metabolic Syndrome is associated with an increased the risk of coronary heart disease and other disorders related to the accumulation of vascular plaque, such as stroke and peripheral vascular disease, referred to as atherosclerotic cardiovascular disease (ASCVD). Patients with Metabolic Syndrome may progress from an insulin resistant state in its early stages to full blown type II diabetes with further increasing risk of ASCVD. Without intending to be bound by any particular theory, the relationship between insulin resistance, Metabolic Syndrome and vascular disease may involve one or more concurrent pathogenic mechanisms including impaired insulin-stimulated vasodilation, insulin resistance-associated reduction in NO availability due to enhanced oxidative stress, and abnormalities in adipocyte-derived hormones such as adiponectin (Lteif and Mather, Can. J. Cardiol. 20 (suppl. B):66B-76B (2004)).

According to the 2001 National Cholesterol Education Program Adult Treatment Panel (ATP III), any three of the following traits in the same individual meet the criteria for Metabolic Syndrome: (a) abdominal obesity (a waist circumference over 102 cm in men and over 88 cm in women); (b) serum triglycerides (150 mg/dl or above); (c) HDL cholesterol (40 mg/dl or lower in men and 50 mg/dl or lower in women); (d) blood pressure (130/85 or more); and (e) fasting blood glucose (110 mg/dl or above). According to the World Health Organization (WHO), an individual having high insulin levels (an elevated fasting blood glucose or an elevated post meal glucose alone) with at least two of the following criteria meets the criteria for Metabolic Syndrome: (a) abdominal obesity (waist to hip ratio of greater than 0.9, a body mass index of at least 30 kg/m2, or a waist measurement over 37 inches); (b) cholesterol panel showing a triglyceride level of at least 150 mg/dl or an HDL cholesterol lower than 35 mg/dl; (c) blood pressure of 140/90 or more, or on treatment for high blood pressure). (Mathur, Ruchi, “Metabolic Syndrome,” ed. Shiel, Jr., William C., MedicineNet.com, May 11, 2009).

For purposes herein, if an individual meets the criteria of either or both of the criteria set forth by the 2001 National Cholesterol Education Program Adult Treatment Panel or the WHO, that individual is considered as afflicted with Metabolic Syndrome.

With regard to the methods of the invention, in some embodiments, the subject suffers from diabetes or obesity or suffers from both diabetes and obesity, in addition to diastolic dysfunction.

In some embodiments, the subject does not suffer from a cardiac injury or a structural heart disease other than the diastolic dysfunction or heart failure being treated or prevented by the inventive method. By “cardiac injury” is meant a disruption of normal cardiac myocyte membrane integrity resulting in the loss into the extracellular space or intracellular constituents including detectable levels of biologically active cytosolic and structure proteins (e.g., troponin, creatine kinase, myoglobin, heart-type fatty acid binding protein, lactate dehydrogenase). By “structural heart disease” is meant any disease that affects the heart muscle or changes the architecture of the heart. In some aspects, the subject does not suffer from ischemic heart disease, chronic stable angina, chronic angina. In some aspects, the subject does not suffer from ischemia, ischemia-reperfusion or coronary artery occlusion-reperfusion, ischemic heart disease, myocardial injury, myocardial toxicity, myocardial infarction, congenital heart lesion, valvular stenosis or valvular regurgitation, coronary artery disease, chronic angina, chronic stable angina, arrhythmias. In some aspects, the subject does not suffer from a myocardial trauma, a myocardial toxicity, a viral infection, a deficiency in nutrients. In some aspects, the subject does not suffer from myocarditis.

In some aspects, the subject does not suffer from the cardiac injury or structural heart disease at the time of administration of the cardiac metabolic modifier. In some aspects, the subject has never experienced the cardiac injury or structural heart disease, or experienced the cardiac injury or structural heart disease at least one year prior to administration of the cardiac metabolic modifier. In some aspects, the subject experienced the cardiac injury or structural heart disease at least two, three, four or five years prior to administration of the cardiac metabolic modifier. In some aspects, the subject experienced the cardiac injury or structural heart disease more than five years (e.g., more than 10 years) prior to administration of the cardiac metabolic modifier.

In some embodiments, the subject suffers from diastolic dysfunction but demonstrates signs of having a structurally normal heart. In some aspects, the heart appears to be structurally normal. In exemplary embodiments, the subject exhibits neither cardiac wall thinning nor a regional wall motion abnormality.

In some aspects, the subject is female, e.g., a female human. In some aspects, the subject is greater than 40 years old, e.g., greater than 45 years old, greater than 50 years old, greater than 55 years old, greater than 60 years old, greater than 65 years old, greater than 70 years old, greater than 75 years old, greater than 80 years old, greater than 85 years old, greater than 90 years old, greater then 95 years old.

The following examples are given merely to illustrate the present invention and not in any way to limit its scope.

### EXAMPLES

### Example 1

The following methods were executed during the studies described below:

Generation of DOCA-salt mouse model: Previously, it was shown that this model leads to mild hypertension, myocardial oxidative stress, and diastolic dysfunction.²⁰ A gradual and mild elevation in blood pressure was induced by unilateral nephrectomy, subcutaneous implantation of a controlled release deoxycorticosterone acetate (DOCA) pellet (0.7 mg/d; Innovative Research of America, Sarasota, Fla.), and substituting drinking water with 1.05% saline. Control animals underwent a sham operation, had placebo pellet implantation, and received water without salt.

Invasive hemodynamic studies, noninvasive echocardiography, and myocyte isolation were done on postoperative day 14-18 for DOCA-salt and control mice. All experiments were approved by the University of Illinois at Chicago Animal Care and Use Committee.

Noninvasive assessment of diastolic dysfunction: Mice were anesthetized, maintained at 37° C., and studied by echocardiography (Vevo 770, VisualSonics Inc, Toronto, Canada). M-mode images in the parasternal long axis and the left ventricle (LV) short-axis views at the mid-papillary level. Measurements were averaged from three consecutive beats during expiration. LV inflow velocities (E and A waves) were interrogated by conventional pulsed-wave Doppler from the apical four-chamber view. The mitral annulus longitudinal velocities (Sm, E′, and A′) were determined by pulsed-wave tissue Doppler from the apical four-chamber view. Interpretation was done by two investigators blinded to the treatment groups. First, baseline images were acquired. Subsequently, the mice were injected with 30 mg/kg ranolazine by intraperitoneal route, followed by a second echocardiogram 30 min later.

Invasive assessment of diastolic dysfunction: Mice were anesthetized with 1-1.5% isoflurane and maintained at 37° C. The pressure-volume (PV) catheter was inserted into the right common carotid artery and advanced into the LV. Inferior vena cava occlusion was performed via a midline abdominal incision. Volume and parallel conductance calibration were performed as previously described.²⁰ Baseline hemodynamic measurements were obtained, and subsequently, the mice received an intravenous injection of ranolazine (5 mg/kg) followed by an infusion at 4.8 mg/kg/h, while additional hemodynamic measurements were recorded. Blood samples were obtained during the last five minutes of the procedure to determine the plasma ranolazine concentration.

Myocyte isolation: Cardiac ventricular myocytes were isolated from the hearts of DOCA-salt or age matched controls mice 14-18 d post-operatively using a modified enzymatic digestion protocol from the Alliance for Cellular Signaling as previously described.²⁰

Cell shortening and calcium transient measurements: The mechanical properties of the cardiomyocytes were assessed using an IonOptix Myocam System (Ionoptix Inc., Milton, Mass.). Unloaded cardiomyocytes were placed on a glass slide and allowed to adhere for 10 min. Cardiomyocytes were then imaged with an inverted microscope and perfused with a Tyrode's buffer containing 1.2 mmol/L calcium at room temperature. Cardiomyocytes were paced at 0.5 Hz for 10 ms duration, and sarcomere shortening and relengthening were assessed using the following indices: peak fractional shortening (FS), time to 90% peak shortening, and τ, the relaxation time constant (a₀+a₁e^{t/τ}, t=time). Cardiomyocytes were treated with 10 μmol/L ranolazine for 10 min prior to evaluation. For calcium transient measurements, cardiomyocytes were loaded with 1 μmol/L Fura 2-AM for 10 min at room temperature, and fluorescence measurements were recorded with a dual-excitation fluorescence photomultiplier system (IonOptix). After loading, the cells were washed and resuspended in normal Tyrode solution. The cardiomyocytes were placed then in the cell chamber, stimulated at 0.5 Hz for 10 ms duration, and imaged through a Fluor×40 objective lens. Cells were exposed to light emitted by a 75-W Xenon lamp and passed though either a 340- or 380-nm wavelength filter. The emitted fluorescence was detected at 510 nm. To take into account any inference from background fluorescence, the background fluorescence for each cardiomyocyte was determined by moving the cardiomyocyte out of the view and recording the fluorescence from the bath solution alone.

Electrophysiological determination of sodium current: Voltage-clamp experiments were performed on isolated murine ventricular myocytes with an Axopatch 200B amplifier (Molecular Devices, Sunnyvale, Calif.) in whole cell configuration. Data acquisition was performed at a sampling rate of 20 kHz and filtered at 10 kHz. Data recording and analysis were done with the pClamp8 software suite (Molecular Devices) and OriginPro 8 (Originlab, Northampton, Mass.). All experiments were carried out at room temperature. Myocytes were plated on glass cover slips and were perfused with a low-sodium Tyrode solution containing the following (in mM): N-methyl-D-glucamine 100 (titrated to pH 7.4 with HCl), NaCl 15, tetramethylammonium chloride 20, CsC15, MgCl₂ 1, glucose 10, 4-aminopyridine 3, MnCl₂ 2, HEPES10, and CaCl₂ 1 (final pH 7.4, CsOH). Patch electrodes were pulled from capillaries purchased from Harvard Apparatus (Holliston, Mass.) using a model P-97 puller from Sutter Instruments (Novato, Calif.). Electrodes were filled with an electrode solution containing (in mM): CsCl 20, tetraethylammonium chloride 20, glutamic acid 80, NaCl 10, MgCl₂ 1, MgATP 5, Li₂GTP 0.3, HEPES10, EGTA 10, CaCl₂ 0.13 (corresponding to [Ca²⁺]_{free} of <10 nM).²³ Electrode solution pH was adjusted to 7.2 with CsOH. Electrodes used for these experiments had access resistances between 1.0 and 1.5 MΩ.

Ranolazine was provided as a crystalline solid by Gilead Sciences. Prior to experiments, a DMSO stock solution was prepared and diluted (minimum 100:1) directly into the Tyrode solution used for perfusion. Cells that were treated with ranolazine were exposed to the drug for 15 min prior to beginning voltage-clamp experiments.

Ca²⁺-sensitivity of tension in skinned fibers: Myofilament response to Ca²⁺ was measured as essentially described in Reference 24. Left ventricular papillary muscles were dissected from mouse hearts and placed in 4° C. high relaxing solution (HR), which contained 10.00 mM EGTA, 41.89 mM K-Prop, 6.57 mM MgCl₂, 100.00 mM 2-[bis(2-hydroxyethyl)amino]ethanesulfonic acid (BES), 6.22 mM Na-ATP, 10.00 mM CrP, 5.00 mM Na-azide, 2.5 μg/mL Pepstatin, 1 μg/mL Leupeptin, and 50 μM PMSF, pH 7.0. The muscles were then dissected further under the microscope into uniform thin bundles of about 3-5 mm in length and 150-250 μm in diameter. The fiber bundles were placed then into a skinning solution of HR that contained 1% triton for 1-2 h at 4° C. The skinned fiber bundles were mounted with cellulose acetate glue in a force measuring apparatus and sarcomere length was adjusted to 2.2 μm using a laser diffraction pattern and width and thickness were determined for calculation of cross-sectional area. The fiber bundles were incubated in ranolazine or vehicle for 15 minutes, and then were activated at 22° C. over a series of pCa (−log of the M Ca²⁺ concentration) values between pCa 8.00 and pCa 4.5. Activating solutions were prepared by mixing HR with a pCa 4.50 solution of 10.00 mM EGTA, 9.99 mM CaCl₂, 22.16 mM K-Prop (K-Prop stock had 1.00 M propionic acid and 1.00 M KOH), 6.20 mM MgCl₂, 100.00 mM BES, 6.29 mM Na-ATP, 10.00 mM creatine phosphate (CrP), 5.00 mM Na-azide, 2.5 μg/mL pepstatin, 1 μg/mL leupeptin, and 50 μM PMSF, pH 7.0. HR and pCa 8.00-pCa 4.50 solutions had one unit of creatine phosphokinase per 200 μL of solution. Tension generated was computed from the force/cross-sectional area and data were analyzed in the Graphpad Prism software and fit into a sigmoidal modified Hill equation in order to generate tension-pCa curves, Hill coefficients, and pCa₅₀ (pCa value at half-maximum tension).

Statistical analysis: Each value is expressed as mean±SE. A two-way ANOVA was used to test for mean differences in invasive and noninvasive parameters. A one-way ANOVA was used to test for mean differences in all other experiments. Where appropriate, post hoc ANOVA testing (Tukey's) was used to assess mean differences between groups at a given time point. A p value <0.05 was considered significant.

### Example 2

Ranolazine attenuated diastolic dysfunction in vivo. As previously described, DOCA-salt mice had evidence of diastolic dysfunction with preserved systolic function by transthoracic echocardiography at postoperative days 14-18 (Table 1).²⁰ Intraperitoneal injection of ranolazine improved diastolic dysfunction without affecting systolic function. DOCA-salt mice had significant reductions in tissue mitral annulus early longitudinal (E′) velocities and the ratio of early annulus to late annulus (E′/A′) velocities, which improved to sham levels with ranolazine treatment (FIG. 1). The ratio of early diastolic filling velocity to the early diastolic mitral annulus velocity (E/E′) has been reported to have the highest correlation with invasive hemodynamic measures of diastolic dysfunction. 20, 25 Hypertensive mice had a higher E/E′ compared to controls, and ranolazine returned this ratio toward normal in hypertensive mice. The mitral inflow velocities, E and A, were similar among the groups, a pseudonormal pattern, as reported before.²⁰ The changes in relaxation parameters occurred in the absence of valvular regurgitation, LV wall motion abnormalities, or hypertrophy. Systolic function including LVEF (%), fractional shortening (FS, %), and septal annulus systolic velocity (Sm) were statistically indistinguishable among the groups (Table 1).

TABLE 1
Effect of ranolazine on echocardiographic parameters

| | | Sham + | | DOCA-salt + |
|---|---|---|---|---|
| | Sham | ranolazine | DOCA-salt | ranolazine |
| FS (%) | 52.6 ± 1.5 | 52.8 ± 0.8 | 49.6 ± 1.3 | 46.9 ± 1.6 |
| Sm (cm/s) | 2.45 ± 0.08 | 2.31 ± 0.09 | 2.18 ± 0.15 | 2.15 ± 0.14 |
| E/A | 1.47 ± 0.05 | 1.63 ± 0.07* | 1.21 ± 0.10 | 1.62 ± 0.11* |
| E′ (cm/s) | 2.77 ± 0.15* | 2.67 ± 0.11* | 2.15 ± 0.11 | 2.47 ± 0.18 |
| E′/A′ | 1.22 ± 0.06* | 1.16 ± 0.02* | 0.82 ± 0.06 | 1.31 ± 0.11* |
| E/E′ | 31.9 ± 2.8* | 30.2 ± 1.9* | 41.8 ± 2.6 | 31.9 ± 2.6* |

Data are means ± SEM.
FS, fractional shortening;
Sm, systolic septal mitral annulus velocity measured by tissue doppler imaging (TDI);
E, early diastolic filling velocity and
A, late diastolic filling velocity measured by conventional doppler;
E′, early septal mitral annulus velocity (TDI);
A′, late diastolic septal mitral annulus velocity (TDI).
n = 8-10,
*p < 0.05 vs. DOCA.

Invasive hemodynamic evaluation confirmed the echocardiographic findings (Table 2). As expected, systolic blood pressure (SBP), diastolic blood pressure (DBP), and left ventricle (LV) end-systolic pressure were mildly elevated in DOCA-salt mice compared with sham and sham treated mice, although DOCA-salt mice treated with ranolazine did not differ significantly from DOCA-salt mice in any of these parameters. As described before, the best fit for the end-diastolic pressure-volume relation (EDPVR) was by the following linear function: pressure_{end diastole}=EDPVR×volume_{end diastole}+intercept (Table 2, FIG. 2A).²⁰ Hypertensive DOCA-salt mice had a steeper EDPVR compared with DOCA-salt treated and control groups (p<0.005; FIGS. 2A and 2B). The slopes were 0.23±0.026, 0.17±0.01, 0.16±0.01, and 0.18±0.01 mm Hg/μL; for DOCA-salt, DOCA-salt+ranolazine, sham, and sham+ranolazine, respectively. Additionally, the EDPVR in DOCA-salt mice demonstrated a linear response to serum ranolazine levels (correlation coefficient=0.70, p<0.05; FIG. 2C).

TABLE 2
Effect of ranolazine on hemodynamics

| | | Sham + | | DOCA-salt + |
|---|---|---|---|---|
| | Sham | ranolazine | DOCA-salt | ranolazine |
| SBP (mmHg) | 88 ± 3* | 93 ± 3* | 109 ± 6 | 102 ± 3 |
| DBP (mmHg) | 52 ± 5* | 58 ± 2* | 78 ± 4 | 73 ± 2 |
| HR (bpm) | 618 ± 24 | 620 ± 5 | 588 ± 16 | 588 ± 16 |
| LVESP (mmHg) | 83 ± 2* | 84 ± 3* | 107 ± 7 | 98 ± 3 |
| LVEDP (mmHg) | 3.2 ± 0.4 | 4.2 ± 0.5 | 5.1 ± 0.9 | 3.6 ± 0.4 |
| EF (%) | 71 ± 5 | 73 ± 5 | 67 ± 6 | 67 ± 4 |
| EDPVR (mmHg/μL) | 0.16 ± 0.01* | 0.18 ± 0.01* | 0.23 ± 0.026 | 0.17 ± 0.01* |

Data are means ± SEM.
SBP, systolic blood pressure;
DBP, diastolic blood pressure;
HR, heart rate;
LVESP, left ventricular end systolic pressure;
LVEDP, left ventricular end diastolic pressure;
EF, ejection fraction;
EDPVR, end diastolic pressure volume relationship.
n = 8,
*p < 0.05 vs. DOCA-salt.

### Example 3

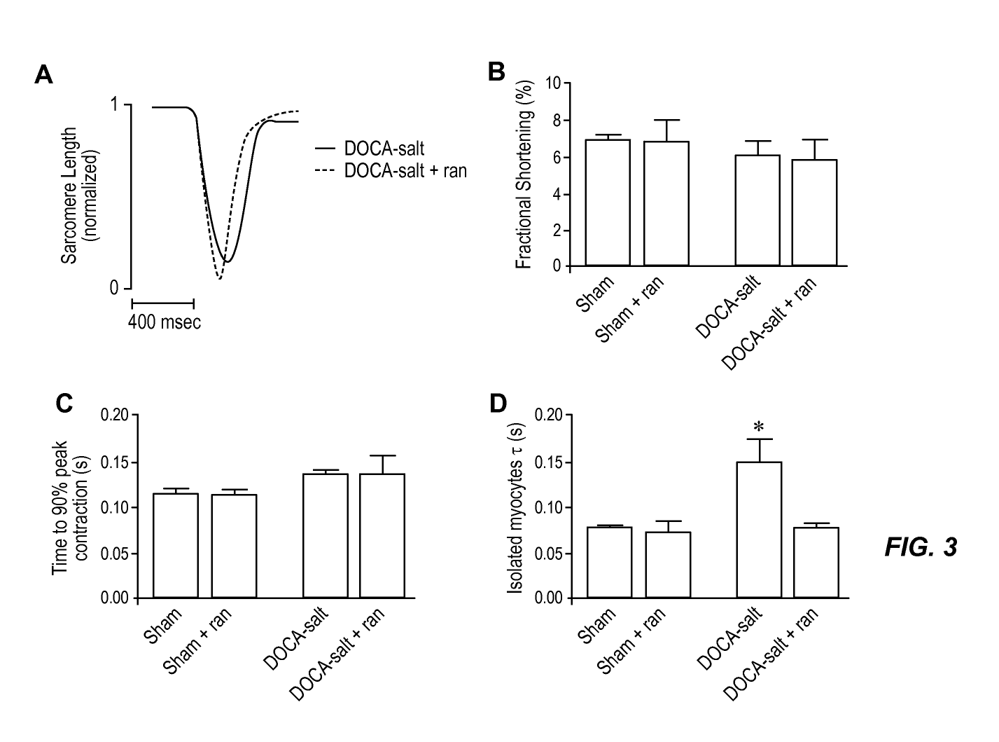

Ranolazine improved relaxation in DOCA-salt cardiomyocytes. Silberman et al. showed that impaired relaxation of cardiomyocytes was the main contributor to diastolic dysfunction in the DOCA-salt hypertensive model, finding no increase in cardiac fibrosis or inflammation.²⁰ To confirm that ranolazine was working directly on DOCA-salt cardiomyocytes to improve relaxation, freshly isolated ventricular cardiomyocytes were studied. DOCA-salt cardiomyocytes had preserved contractile function, as previously reported (FIGS. 3A, 3B, and 3C).²⁰ Additionally, treatment with ranolazine did not affect contraction in sham and DOCA-salt mice (FIGS. 3A, 3B, and 3C). In contrast, relaxation τ was significantly impaired in DOCA-salt mice and improved to normal levels with ranolazine treatment (DOCA-salt 0.15±0.03, DOCA-salt+ranolazine 0.08±0.01, sham 0.08±0.01, sham+ranolazine 0.07±0.01 seconds, p<0.0001; FIGS. 3A and 3D).

### Example 4

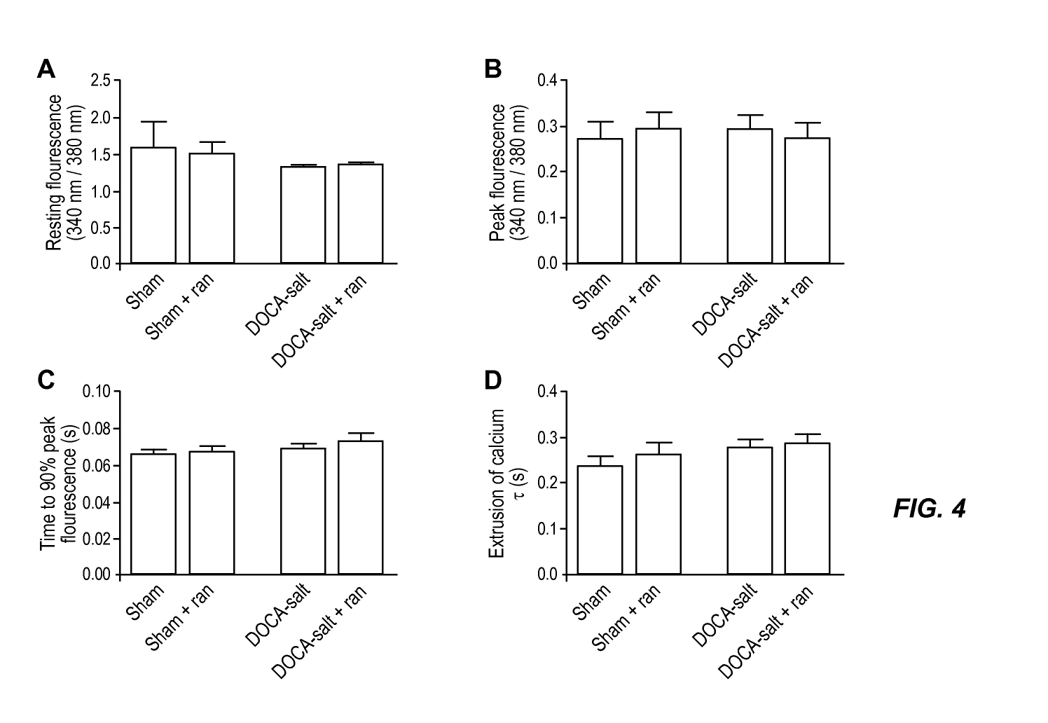

Diastolic dysfunction was independent of intracellular calcium cycling. To elucidate the mechanism underlying the impaired diastolic function in hypertensive mice, Ca²⁺ transients were measured in freshly isolated ventricular myocytes. Ca²⁺ transients did not differ significantly between sham and DOCA-salt mice, and the addition of 10 μM ranolazine did not affect either group (FIG. 4). Baseline intracellular Ca²⁺ was similar in all groups (FIG. 4A). Additionally, peak intracellular Ca²⁺ and the rate of Ca²⁺ release were similar between all four groups (FIGS. 4B and 4C). Surprisingly, there was no difference among the rates of intracellular Ca²⁺ egress among the four groups measured as the time constant τ (FIG. 4D).

### Example 5

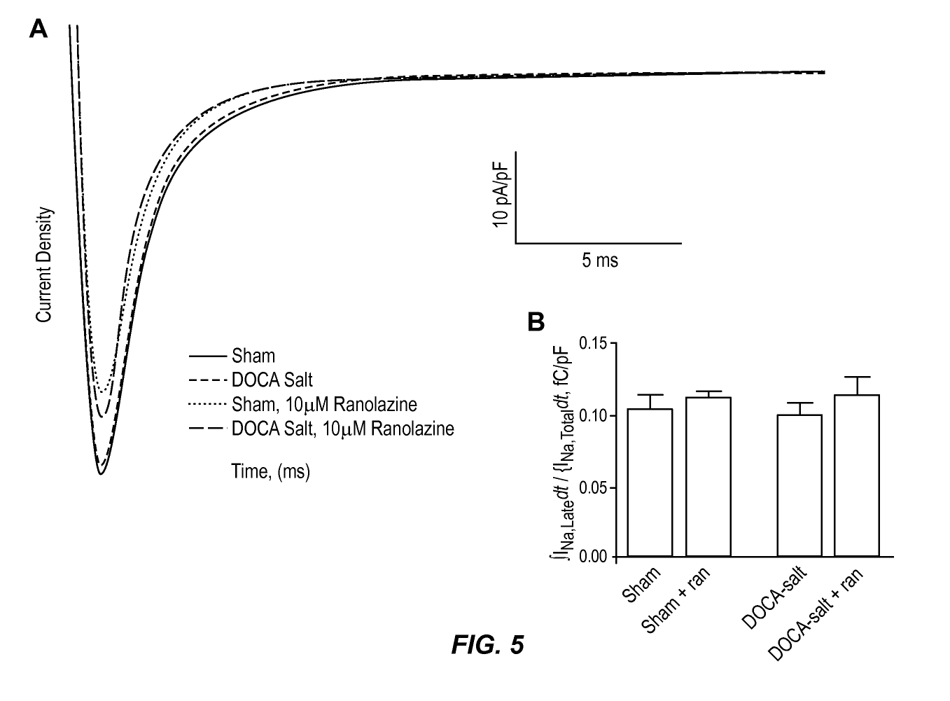

Late I_{Na} was not elevated in DOCA-salt mice. Oxidative stress is known to induce late I_{Na} that can be blocked by the anti-anginal drug, ranolazine, and the DOCA-salt model is associated with increased cardiac oxidative stress.^{20,26} Nevertheless, voltage-clamp studies show no increase in late I_{Na} in DOCA-salt cardiomyocytes compared to sham (sham 0.10532±0.00999, sham+ranolazine 0.11257±0.00406, DOCA-salt 0.10088±0.00792, DOCA-salt+ranolazine 0.11422±0.01272, p=NS) (FIG. 5A). Integrated late I_{Na} was measured starting at 5% of peak current and ending 40 ms after depolarization. DOCA-salt myocytes accumulated a similar amount of charge as sham. Extracellular addition of 10 μmol/L ranolazine did not affect the late accumulated charge in DOCA-salt cardiomyocytes, which was similar to that seen for sham and sham treated myocytes (FIG. 5B). Peak I_{Na} was similar among all four groups.

### Example 6

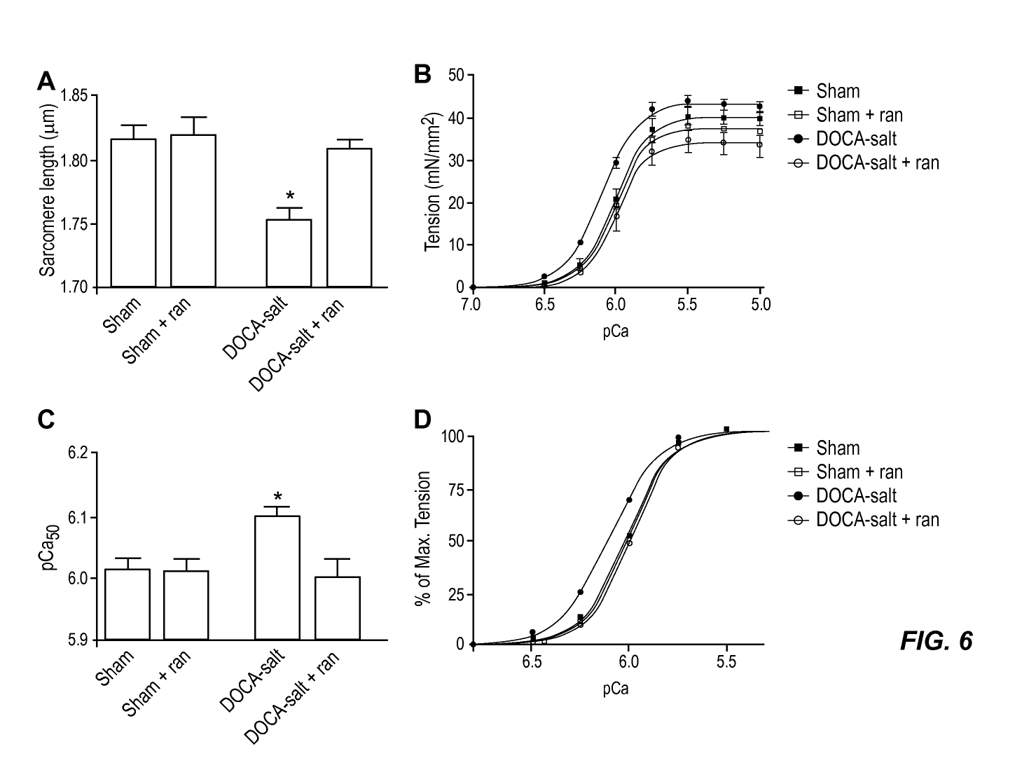

DOCA-salt cardiomyocytes demonstrated increased myofilament Ca²⁺ sensitivity The baseline sarcomere length was significantly reduced in DOCA-salt mice compared to sham mice (FIG. 6A). Treatment with ranolazine improved resting sarcomere length in the DOCA-salt mice to levels similar to sham, and ranolazine did not affect sham sarcomere length (DOCA-salt 1.75±0.01, DOCA-salt+ranolazine 1.81±0.01, sham 1.82±0.01, sham+ranolazine 1.82±0.01 μm, p<0.0001). In the absence of changes in resting Ca²⁺, this suggested that myofilament Ca²⁺ sensitivity may be altered in diastolic dysfunction. To examine directly myofilament function, the calcium sensitivity of steady-state isometric force development was measured in skinned sham and DOCA-salt cardiomyocytes. The mean steady-state isometric tension and normalized tension plotted as a function of calcium in skinned ventricular myofibers showed the DOCA-salt curve shifted to the left representing an increase in Ca²⁺ sensitivity (FIGS. 6B and 6D). Myofilament Ca²⁺ sensitivity, indexed by pCa₅₀, was significantly (p<0.02) greater in DOCA-salt than in sham cardiomyocytes (FIG. 6C). Ranolazine treatment returned DOCA-salt myofilament sensitivity to levels similar to sham myocytes with little effect on sham myocytes.

### DISCUSSION OF THE FOREGOING EXAMPLES

The effects of ranolazine, a late I_{Na} inhibitor, on the mechanical derangements induced in the DOCA-salt hypertensive model of diastolic dysfunction^{26,27} were investigated. Mild hypertension in this model resulted in impaired relaxation that improved acutely after ranolazine treatment. Without changes in heart rate or blood pressure, ranolazine rapidly improved relaxation when measured both noninvasively and invasively. EDPVR, the most widely accepted measurement of relaxation, significantly improved in the DOCA-salt mice confirming our noninvasive echocardiographic results. Additionally, ranolazine did not significantly affect hemodynamics in the sham mouse. At the cellular level, isolated DOCA-salt cardiomyocytes demonstrated impaired relaxation that improved with ranolazine.

Despite the efficacy of ranolazine in the relief of diastolic dysfunction, no increase in late I_{Na} in the DOCA-salt cardiomyocytes was noted nor were there changes in calcium cycling to indicate significant alterations in Ca²⁺ handling in this form of diastolic dysfunction. On the other hand, at rest, the DOCA-salt myocytes had a significant decrease in sarcomere length in the absence of changes in Ca²⁺ concentration, suggesting increased diastolic tension compared to sham mice. Myofilaments from DOCA-salt mice had increased sensitivity to Ca²⁺ compared to sham mice that normalized with ranolazine treatment. Taken together, these results suggest ranolazine improved diastolic function at the cardiomyocyte level through the modulation of myofilament sensitivity to Ca²⁺.

Silberman et al. reported the DOCA-salt model of diastolic dysfunction was associated with cardiac oxidative stress and targeting of reactive oxygen species production improved diastolic function.²⁰ The downstream mediators of the increased oxidant load were investigated to better elucidate the mechanisms regulating diastolic function. Oxidative stress is known to modulate a number of proteins important in cardiac function including: the SR Ca²⁺ release channel, SR Ca²⁺ pump, the sarcolemmal L-type Ca²⁺ channel, the sodium-calcium exchanger, phospholamban, myofilaments, and the late I_{Na}.^{15,28-32} Largely based on analogy to systolic dysfunction, it was expected to find increased diastolic Ca²⁺ resulting in slowed myocyte relaxation and diastolic dysfunction, but surprisingly and unexpectedly, no changes in Ca²⁺ cycling between DOCA-salt and sham mice were found. Instead, changes in myofilament sensitivity in the absence of changes in Ca²⁺ handling were noted. This is consistent with other models of diastolic dysfunction including a model of diabetic cardiomyopathy in which similar changes in sarcomere length were reported with no correlation with changes in Ca²⁺ cycling.¹⁶⁻¹⁸ Previously, it was reported that myocyte relaxation can be dissociated from the decline of intracellular Ca^{2+,33} and myofilament Ca²⁺ sensitivity is a consistent functional abnormality seen in dilated cardiomyopathy.³⁴ These results suggest that although oxidative stress is associated with both systolic and diastolic dysfunction, mediators of this dysfunction appear to differ. This helps explain the unimpressive results treating diastolic dysfunction when using drugs proven to be beneficial in systolic heart failure.⁶⁻⁸

In vitro studies demonstrating the effectiveness of ranolazine to treat impaired relaxation have used isolated muscle strips, isolated cardiomyocytes, and working heart preparations.^{35,36} Previous studies in both rabbit and rat models have shown that ranolazine attenuates diastolic dysfunction in ischemia/reperfusion.^{37,38} Additionally, in a dog model of chronic heart failure, ranolazine reduces LVEDP.³⁹⁻⁴¹ Finally, trials in humans with ischemic heart disease and type-3 long-QT syndrome have supported a role for ranolazine in the treatment of diastolic dysfunction.^{42,43} The mechanism for this effect was consistent with a reduction in late I_{Na} with a subsequent reduction in diastolic Ca²⁺. Nevertheless, we did not find any changes in Ca²⁺ handling with acute ranolazine treatment, despite the drug's efficacy to improve diastolic dysfunction parameters. Instead, it appears that ranolazine may work by altering myofilament Ca²⁺ sensitivity. At this point, it is unclear if this is a direct or indirect effect, but the changes in myofilament Ca²⁺ sensitivity are consistent with a potential antiarrhythmic effect of this drug.⁴⁴

In addition to being a potent inhibitor of late I_{Na}, ranolazine has also been shown to inhibit fatty acid oxidation and the potassium channel, I_{Kr.}^{45,46} Interestingly, in a diabetic cardiomyopathy model in which transgenic mice overexpressing the fatty acid transport protein (FATP) have increased fatty acid uptake, the animals develop diastolic dysfunction with similar changes in sarcomere length and myofilament sensitivity.¹⁶ It is possible that ranolazine can inhibit fatty acid oxidation, limiting toxic metabolites, and preserving diastolic function. Clinical studies have shown ranolazine to have a positive effect of glycemic control providing evidence of a metabolic target in vivo.^{44,47}

In conclusion, the present study demonstrates that ranolazine treatment improves diastolic function through modulation of myofilament sensitivity to calcium. These results suggest that ranolazine may be of value in the treatment of diastolic dysfunction in the absence of systolic dysfunction.

### Example 7

This example provides a revised presentation of some of the experiments and data of Examples 1-6, as well as a description of new experiments.

In this example, the following methods were carried out as follows:

Generation of DOCA-Salt Mouse Model:

Previously, we have shown that this model leads to mild hypertension, myocardial oxidative stress, and diastolic dysfunction.²⁰ A gradual and mild elevation in blood pressure was induced by unilateral nephrectomy, subcutaneous implantation of a controlled release deoxycorticosterone acetate (DOCA) pellet (0.7 mg/d; Innovative Research of America, Sarasota, Fla.), and substituting drinking water with 1.05% saline. Control animals underwent a sham operation, had placebo pellet implantation, and received water without salt.

Invasive hemodynamic studies, noninvasive echocardiography, and myocyte isolation were done on postoperative day 14-18 for DOCA-salt and control mice. All experiments were approved by the University of Illinois at Chicago Animal Care and Use Committee.

Chronic Administration of Ranolazine:

A special diet contained 5 mg ranolazine, 0.3 mg P450 inhibitor, and 0.25 mg red food color was pressed into a 1-g nutritionally complete grain-based tablet (Harlan, Madison, Wis.). The control diet contained 0.3 mg P450 inhibitor and 0.25 mg yellow food color pressed into the same type of 1-g tablets. DOCA-salt and sham mice consumed daily ˜1 g of the special diet from postoperative day 11 to 18.

Noninvasive Assessment of Diastolic Dysfunction:

Mice were anesthetized, maintained at 37° C., and studied by echocardiography (Vevo 770, VisualSonics Inc, Toronto, Canada). M-mode images in the parasternal long axis and the left ventricle (LV) short-axis views at the mid-papillary level were taken. Measurements were averaged from three consecutive beats during expiration. LV inflow velocities (E and A waves) were interrogated by conventional pulsed-wave Doppler from the apical four-chamber view. The mitral annulus longitudinal velocities (Sm, E′, and A′) were determined by pulsed-wave tissue Doppler from the apical four-chamber view. Interpretation was done by a two investigators blinded to the treatment groups. First, baseline images were acquired. Subsequently to determine the acute effect, the mice were injected with 30 mg/kg ranolazine by intraperitoneal route, followed by a second echocardiogram 30 min later.

Invasive Assessment of Diastolic Dysfunction:

Mice were anesthetized with 1-1.5% isoflurane and maintained at 37° C. The pressure-volume (PV) catheter was inserted into the right common carotid artery and advanced into the LV. Inferior vena cava occlusion was performed via a diaphragm incision. Volume and parallel conductance calibration were performed as previously described.²⁰ Baseline hemodynamic measurements were obtained, and subsequently to determine the acute effect, the mice received an intravenous injection of ranolazine (5 mg/kg) followed by an infusion at 4.8 mg/kg/h, while additional hemodynamic measurements were recorded. Blood samples were obtained during the last five minutes of the procedure to determine the plasma ranolazine concentration.

Cell Shortening and Calcium Transient Measurements:

The mechanical properties of the cardiomyocytes were assessed using an IonOptix Myocam System (Ionoptix Inc., Milton, Mass.). Unloaded cardiomyocytes were placed on a glass slide and allowed to adhere for 10 min at 37° C. Cardiomyocytes were then imaged with an inverted microscope and perfused with a Tyrode's buffer containing 1.2 mmol/L calcium at room temperature. Cardiomyocytes were paced at 0.5, 1, or 2 Hz for 10 ms duration, and sarcomere shortening and relengthening were assessed using the following indices: peak fractional shortening (FS), time to 90% peak shortening, and τ, the relaxation time constant (a₀+a₁e^{t/τ}, t=time). Cardiomyocytes were treated with 10 μmol/L ranolazine for 10 min prior to evaluation. Initial experiments were done at room temperature and subsequent studies at 37° C. showed no change in the effects of DOCA-salt or ranolazine. For calcium transient measurements, cardiomyocytes were loaded with 1 μmol/L fura 2-AM for 10 min at 37° C., and fluorescence measurements were recorded with a dual-excitation fluorescence photomultiplier system (IonOptix). After loading, the cells were washed and resuspended in normal Tyrode solution. The cardiomyocytes were placed then in the cell chamber, stimulated at 0.5, 1, or 2 Hz for 10 ms duration, and imaged through a Fluor×40 objective lens. Cells were exposed to light emitted by a 75-W Xenon lamp and passed though either a 340- or 380-nm wavelength filter. The emitted fluorescence was detected at 510 nm. To take into account any interference, the background fluorescence for each cardiomyocyte was determined by moving the cardiomyocyte out of the view and recording the fluorescence from the bath solution alone.

Studies with Detergent Extracted (Skinned) Fiber Bundles:

We measured myofilament response to Ca²⁺ under two experimental conditions as described in detail below. In a first series of experiments the skinned fiber bundles were mounted in a force measuring apparatus and sarcomere length (SL) was adjusted to 2.2 μm using a laser diffraction pattern and width and thickness were determined for calculation of cross-sectional area. Force was measured over a range of pCa values Skinned fiber bundles were treated with 10 μmol/L ranolazine for 15 min prior to evaluation. In a second series of experiments under different experimental conditions, we determined steady-state Mg-ATPase activity, while simultaneously measuring isometric tension in skinned fiber bundles as a function of pCa. In all experiments, skinned fibers were treated with ranolazine or DMSO vehicle for 15 min prior to beginning either tension or tension/ATPase rate measurements. Tension generated was computed from the force/cross-sectional area and data were analyzed in the GraphPad Prism software and fit with a sigmoidal modified Hill equation in order to generate tension-pCa curves, Hill coefficients, and pCa₅₀ (pCa value at half-maximum tension). We also determined cross-bridge kinetics employing a quick release/restretch protocol. The rate constant for force redevelopment, Ktr, provides a measure of the rate of cross-bridge entry into the force generating state. In all experiments, only fiber bundles retaining more than 80% of their initial maximum tension were included in the analysis. We treated the fibers with ranolazine from a DMSO stock solution or DMSO alone added to relaxing solution for 15 min prior to the experimental run. Methods for gel electrophoresis analysis of myofilament protein phosphorylation and oxidative modifications are provided below.

Myocyte isolation: Cardiac ventricular myocytes were isolated from the hearts of DOCA-salt or age matched controls mice 14-18 d post-operatively using a modified enzymatic digestion protocol from the Alliance for Cellular Signaling as previously described (Silberman et al., Circulation 121: 519-528 (2010)).

Voltage clamping studies: Voltage-clamp experiments were performed on isolated murine ventricular myocytes with an Axopatch 200B amplifier (Molecular Devices, Sunnyvale, Calif.) in whole cell configuration. Data acquisition was performed at a sampling rate of 20 kHz and filtered at 10 kHz. Data recording and analysis were done with the pClamp8 software suite (Molecular Devices) and OriginPro 8 (Originlab, Northampton, Mass.). All experiments were carried out at room temperature. Myocytes were plated on glass cover slips and were perfused with a low-sodium Tyrode solution containing the following (in mM): N-methyl-D-glucamine 100 (titrated to pH 7.4 with HCl), NaCl 15, tetramethylammonium chloride 20, CsC15, MgCl₂ 1, glucose 10, 4-aminopyridine 3, MnCl₂2, HEPES10, and CaCl₂ 1 (final pH 7.4, CsOH). Patch electrodes were pulled from capillaries purchased from Harvard Apparatus (Holliston, Mass.) using a model P-97 puller from Sutter Instruments (Novato, Calif.). Electrodes were filled with an electrode solution containing (in mM): CsCl 20, tetraethylammonium chloride 20, glutamic acid 80, NaCl 10, MgCl₂ 1, MgATP 5, Li₂GTP 0.3, HEPES 10, EGTA 10, CaCl₂ 0.13 (corresponding to [Ca²⁺]_{free} of <10 nM) (Patton et al., Cell Calcium 35: 427-431 (2004)). Electrode solution pH was adjusted to 7.2 with CsOH. Electrodes used for these experiments had access resistances between 1.0 and 1.5 MΩ.

Ranolazine was provided as a crystalline solid by Gilead Sciences. Prior to acute experiments, a DMSO stock solution was prepared and diluted (minimum 100:1) directly into the Tyrode solution used for perfusion. Cells that were treated with ranolazine were exposed to the drug for 15 min prior to beginning voltage-clamp experiments.

Studies with detergent extracted (skinned) fiber bundles: Mice were anesthetized with pentobarbital (50 mg/kg IP), and the hearts were rapidly excised and rinsed in a pH 7.0 ice-cold relaxing solution (HR) composed of (in mM) 10 EGTA, 41.89K-Prop, 6.57 MgCl₂, 100 BES, 6.22 ATP, 5 Na azide, and 10 creatine phosphate. The solution also contained 1 μg/ml leupeptin, 2.5 μg/ml pepstatin A, and 50 μM phenylmethylsulfonyl fluoride. Left ventricular papillary muscles were dissected and fiber bundles were prepared as previously described (Wolska et al., J. Physiol. 536: 863-870 (2001)). The fiber bundles were extracted overnight in relaxing solution plus 1% Triton X-100 at 4° C.

In a first series of experiments the skinned fiber bundles were mounted with cellulose acetate glue in a force measuring apparatus and sarcomere length was adjusted to 2.2 μm using a laser diffraction pattern and width and thickness were determined for calculation of cross-sectional area (de Tombe et al., Circ Res 66: 1239-1254 (1990)). The skinned fibers were activated at 22° C. over a series of pCa (−log of the M Ca²⁺ concentration) values between pCa 8.00 and pCa 4.5. Activating solutions were prepared by mixing HR with a pCa 4.50 solution of 10.00 mM EGTA, 9.99 mM CaCl₂, 22.16 mM K-Prop (K-Prop stock had 1.00 M propionic acid and 1.00 M KOH), 6.20 mM MgCl₂, 100.00 mM BES, 6.29 mM Na-ATP, 10.00 mM creatine phosphate (CrP), 5.00 mM Na-azide, 2.5 μg/mL pepstatin, 1 μg/mL leupeptin, and 50 μM PMSF, pH 7.0. HR and pCa8.00-pCa 4.50 solutions had one unit of creatine phosphokinase per 200 μL of solution.

In a second series of experiments force and ATPase rate were measured simultaneously using methods and an experimental apparatus previously described (de Tombe et al., Circ Res 76: 734-741 (1995); Wolska et al., Circ Res 84: 745-751 (1999)). The fiber bundles were mounted between a force transducer and displacement motor using aluminum T-clips, and the sarcomere length was set to 2.2 μm using He—Ne laser diffraction (Vahebi et al., Circ Res 96: 740-747 (2005)). The width and diameter were each measured at three points along the fiber bundle. Force per cross-sectional area was used to determine tension. The fiber was contracted initially at a saturating calcium concentration (pCa 4.5) and sarcomere length was again adjusted to 2.2 μm. Sarcomere length remained constant throughout the rest of the experiment.

ATPase activity was measured at 20° C. as previously described and calibrated with rapid injections of ADP (0.5 nmol) with a motor-controlled syringe (de Tombe et al., 1990, supra; Wolska et al., 1999, supra). The fiber was placed in relaxing solution for 2 min, then in the preactivation solution for 2-3 min each time before being placed in the activating solution for 1-2 min (until stabilization of force) and then quickly returned to the relaxing solution. Various contraction-relaxation cycles were carried out using different ratios of total calcium concentration to total EGTA concentration. The final contraction was again at a saturating calcium concentration. The relation between Ca²⁺ and tension or ATPase activity was fitted using a modified Hill equation as described previously (Canton et al., Am J Physiol Heart Circ Physiol 286: H870-877 (2004)).

Analysis of Post-Translational Modifications:

Skinned fibers with 1% (v/v) Triton X-100 were solubilized in 15 μL sample buffer (8 M urea, 2 M thiourea, 0.05 M tris pH 6.8, 75 mM DTT, 3% SDS, and 0.05% bromophenol blue) by incubation on a shaker for 30 min followed by two cycles of 10 min incubations in a sonicating bath with 30 seconds vortexing between the incubations (Layland et al., FASEB J 19:1137-1139 (2005)). Samples were heated at 100° C. for three min and after 10 min spin clarification, all of the supernatant was loaded on to a 12% resolving 1D SDS-PAGE gel (Wolska et al., 1999, supra; Hill et al., Methods Enzymol 473: 179-197 (2010)) The gels were stained and destained with Pro-Q Diamond (Invitrogen) according to the manufacturer's recommendations preceding imaging with a Typhoon 9410 scanner (GE Healthcare). Optical density of the proteins was determined using ImageQuant TL (GE Healthcare) software and Commassie R-250 stained gel was used to normalize protein load to both actin the whole lane. Results were exported to Excel and analyzed with JMP statistical software (Cary, N.C.).

Western blot analysis was used to detect for glutathionylated proteins. Myofibrils were prepared from DOCA and sham mouse hearts, and pellets were solubilized in a non-reducing 2×Laemmli buffer (4% SDS, 20% glycerol, 0.004% bromophenol blue, and 0.125 M Tris HCl pH 6.8). 25 mM N-ethylmaleimide (NEM) was added to the standard rigor buffer with Triton X-100, the standard rigor wash buffer and the 2× Laemmli buffer (Layland et al., 2005, supra). Using the protein concentration determined from an RC-DC (Bio-Rad) assay, 40 μg of total protein was applied to 1D 12% resolving SDS-PAGE gel and transferred onto a 0.2 μM Polyvinylidene fluoride (PVDF) membrane. The blot was blocked in 5% nonfat dry milk with 2.5 mM NEM for 1 hour. Anti-glutathione mouse monoclonal primary antibody (Virogen, Watertown, Mass.) was used at 1:1000 dilution along with anti-mouse HRP-conjugated secondary antibody (Sigma) at 1:100,000 dilution to detect for S-glutathionylation (Hill et al., 2010, supra). Optical density of the bands was measured with ImageQuant TL (GE Healthcare) and exported to Excel for further analysis. An antibody to myosin binding protein C was used for identification of the band indentified as being modified.

Statistical Analysis:

Each value is expressed as mean±SE. A one-way ANOVA was used to test for mean differences in invasive and noninvasive parameters. Where appropriate, post hoc ANOVA testing (Tukey's) was used to assess mean differences between groups at a given time point. A p value <0.05 was considered significant.

The following results were obtained:

Ranolazine Attenuated Diastolic Dysfunction In Vivo.

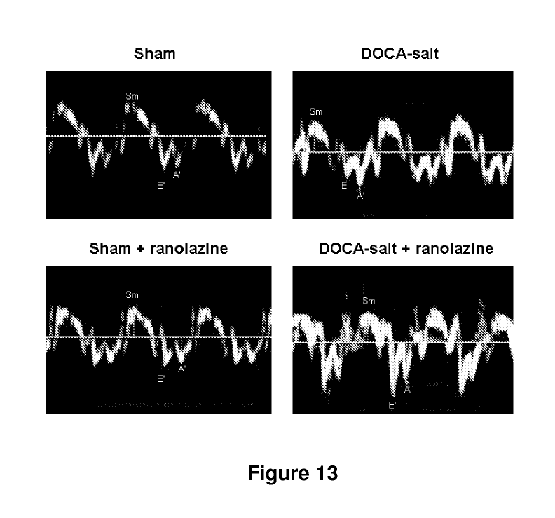

As previously described, DOCA-salt mice had evidence of diastolic dysfunction with preserved systolic function by transthoracic echocardiography at postoperative days 14-18 (table 1).²⁰ Intraperitoneal injection of ranolazine improved diastolic dysfunction without affecting systolic function. DOCA-salt mice had significant reductions in tissue mitral annulus early longitudinal (E′) velocities and the ratio of early annulus to late annulus (E′/A′) velocities, which improved to sham levels with ranolazine treatment (FIG. 13). The ratio of early diastolic filling velocity to the early diastolic mitral annulus velocity (E/E′) has been reported to have the highest correlation with invasive hemodynamic measures of diastolic dysfunction.^{20,23} Hypertensive mice had a higher E/E′ compared to controls, and ranolazine returned this ratio toward normal in hypertensive mice. The mitral inflow velocities, E and A, were similar among the groups, a pseudonormal pattern, as reported before.²⁰ The changes in relaxation parameters occurred in the absence of valvular regurgitation, LV wall motion abnormalities, or hypertrophy. Despite a slight reduction in fractional shortening (FS, %) when comparing sham to DOCA-salt+ranolazine groups, LVEF (%) and septal annulus systolic velocity (Sm) were statistically indistinguishable among the groups, suggesting the treatments had little effect on systolic function (Table 3).

TABLE 3

| | | Sham + | | DOCA-salt + |
|---|---|---|---|---|
| | Sham | ranolazine | DOCA-salt | ranolazine |
| EF (%) | 52.6 ± 1.5† | 52.8 ± 0.8† | 49.6 ± 1.3 | 46.9 ± 1.6 |
| Sm (cm/s) | 2.45 ± 0.08 | 2.31 ± 0.09 | 2.18 ± 0.15 | 2.15 ± 0.14 |
| E/A | 1.47 ± 0.05 | 1.63 ± 0.07* | 1.21 ± 0.10 | 1.62 ± 0.11* |
| E′ (cm/s) | 2.77 ± 0.15* | 2.67 ± 0.11* | 2.15 ± 0.11 | 2.47 ± 0.18 |
| E′/A′ | 1.22 ± 0.06* | 1.16 ± 0.02* | 0.82 ± 0.06 | 1.31 ± 0.11* |
| E/E′ | 31.9 ± 2.8* | 30.2 ± 1.9* | 41.8 ± 2.6 | 31.9 ± 2.6* |

Data are means ± SEM.
EF, ejection fraction;
Sm, systolic septal mitral annulus velocity measured by tissue doppler imaging (TDI);
E, early diastolic filling velocity and
A, late diastolic filling velocity measured by conventional doppler;
E′, early septal mitral annulus velocity (TDI);
A′, late diastolic septal mitral annulus velocity (TDI).
n = 8-10,
*p < 0.05 vs. DOCA-salt.
†p < 0.05 vs. DOCA-salt + ran.

Invasive hemodynamic evaluation confirmed the echocardiographic findings (Table 4).

TABLE 4

| | | Sham + | | DOCA-salt + |
|---|---|---|---|---|
| | Sham | ranolazine | DOCA-salt | ranolazine |
| SBP (mmHg) | 88 ± 3*† | 93 ± 3* | 109 ± 7 | 102 ± 3 |
| DBP (mmHg) | 52 ± 5*† | 58 ± 2*† | 78 ± 4 | 73 ± 2 |
| HR (bpm) | 618 ± 24 | 620 ± 5 | 589 ± 16 | 588 ± 16 |
| LVESP (mmHg) | 83 ± 2* | 85 ± 3* | 108 ± 7 | 98 ± 3 |
| LVEDP (mmHg) | 3.2 ± 0.3* | 4.2 ± 0.5 | 5.1 ± 0.9 | 3.5 ± 0.4* |
| EF (%) | 71 ± 6 | 73 ± 5 | 67 ± 6 | 67 ± 4 |
| EDPVR (mmHg/μL) | 0.16 ± 0.01* | 0.18 ± 0.01* | 0.23 ± 0.02 | 0.17 ± 0.01* |
| ESPVR (mmHg/μL) | 3 ± 0.6 | 3.9 ± 0.5 | 4.7 ± 0.8 | 4.1 ± 0.2 |
| dP/dt min (mmHg/sec) | −10,420 ± 594 | −10,083 ± 685 | −12,181 ± 641 | −12,211 ± 792 |
| dP/dt max (mmHg/sec) | 12,818 ± 490 | 12,032 ± 787 | 13,585 ± 832 | 12,495 ± 555 |

Data are means ± SEM.
SBP, systolic blood pressure;
DBP, diastolic blood pressure;
HR, heart rate;
LVESP, left ventricular end systolic pressure;
LVEDP, left ventricular end diastolic pressure;
EF, ejection fraction;
EDPVR, end diastolic pressure volume relationship.
ESPVR, end systolic pressure volume relationship.
n = 8,
*p < 0.05 vs. DOCA-salt.
†p < 0.05 vs. DOCA-salt + ranolazine.

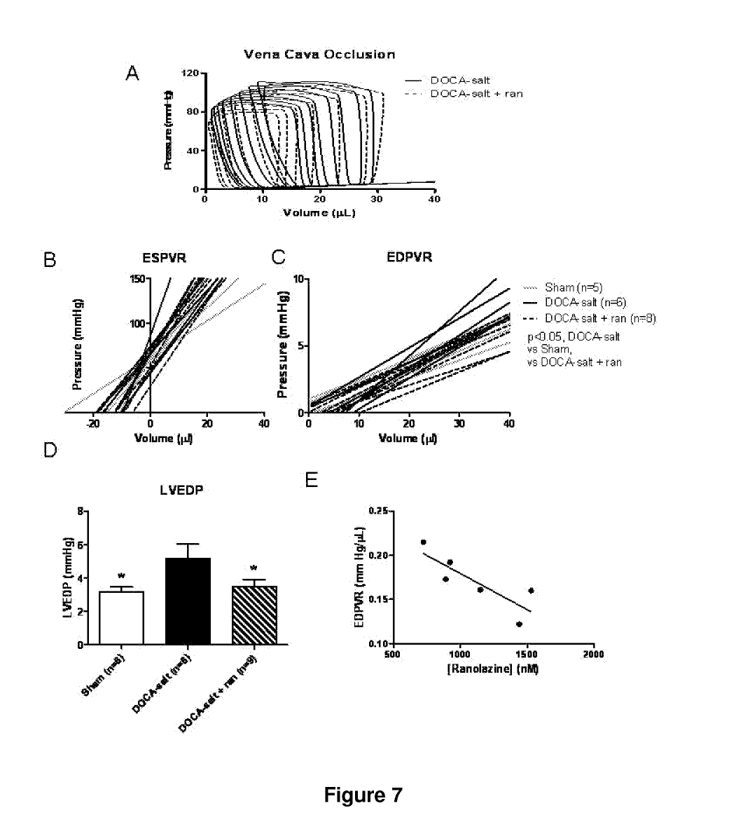

As expected, systolic blood pressure (SBP), diastolic blood pressure (DBP), and left ventricle (LV) end-systolic pressure were mildly elevated in DOCA-salt mice compared with sham and sham treated mice, although DOCA-salt mice treated with ranolazine did not differ significantly from DOCA-salt mice in any of these parameters. As described before, the best fit for the end-diastolic pressure-volume relation (EDPVR) was by the following linear function: pressure_{end diastole}=EDPVR×volume_{end diastole}+intercept.²⁰ Hypertensive DOCA-salt mice had a steeper EDPVR compared with DOCA-salt treated and control groups (p<0.005; FIGS. 7B and 7C). The slopes were 0.23±0.026, 0.17±0.01, and 0.16±0.01 mm Hg/μL; for DOCA-salt, DOCA-salt+ranolazine and sham, respectively. Additionally, the EDPVR in DOCA-salt mice demonstrated a linear response to serum ranolazine levels (correlation coefficient=0.70, p<0.05; FIG. 7E).

In an additional set of experiments, we treated DOCA-salt mice with ranolazine for 7 days and measured diastolic function using echocardiography to evaluate the effect of chronic ranolazine therapy on myocardial function (Table 5).

TABLE 5
Online Table I. Chronic Effect of Ranolazine on
echocardiographic parameters

| | | Sham + | | DOCA-salt + |
|---|---|---|---|---|
| | Sham | ranolazine | DOCA-salt | ranolazine |
| ES (%) | 55.1 ± 4.7 | 49.7 ± 4.9 | 50.8 ± 4.3 | 53.3 ± 1.3 |
| Sm (cm/s) | 2.85 ± 1.87 | 2.04 ± 0.12 | 1.85 ± 0.17 | 1.82 ± 0.05 |
| E/A | 2.31 ± 0.31 | 2.42 ± 0.42 | 3.11 ± 0.85 | 1.84 ± 0.35 |
| E′ (cm/s) | 2.23 ± 0.17* | 2.11 ± 0.21 | 1.43 ± 0.08 | 1.86 ± 0.13* |
| E′/A′ | 1.20 ± 0.09* | 1.03 ± 0.07 | 0.74 ± 0.05 | 1.10 ± 0.08* |
| E/E′ | 30.4 ± 1.17* | 28.0 ± 3.45* | 43.7 ± 2.73 | 29.3 ± 5.07* |

Data are means ± SEM.
EF, ejection fraction;
Sm, systolic septal mitral annulus velocity measured by tissue doppler imaging (TDI);
E, early diastolic filling velocity and
A, late diastolic filling velocity measured by conventional doppler;
E′, early septal mitral annulus velocity (TDI);
A′, late diastolic septal mitral annulus velocity (TDI).
n = 7-10,
*p < 0.05 vs. DOCA-salt.

Prolonged treatment of DOCA-salt mice with ranolazine significantly improved the E′/A′ ratio in DOCA-salt mice (DOCA-salt 0.74±0.05 vs. DOCA-salt+Ran 1.10±0.08, p<0.05). Moreover, ranolazine decreased E/E′ in DOCA-salt mice when treated chronically (DOCA-salt 43.69±2.73 vs. DOCA-salt+Ran 29.27±5.07, p<0.05). Prolonged ranolazine treatment did not affect the systolic function (Sₘ) in any of the groups.

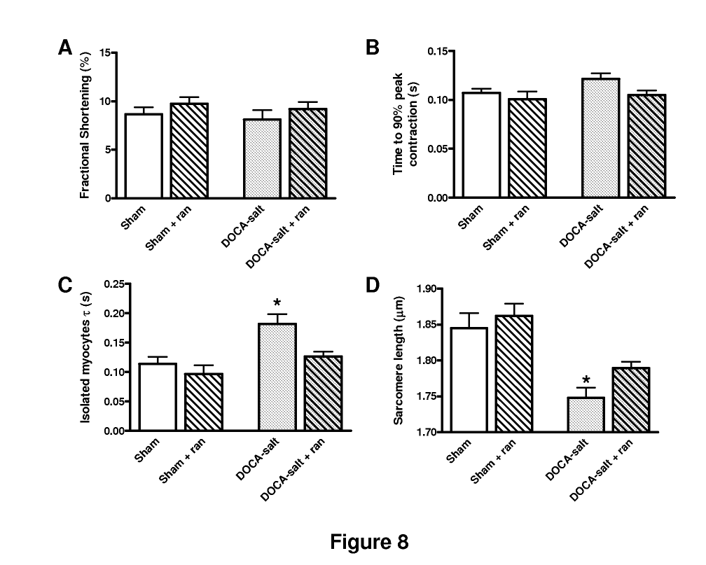

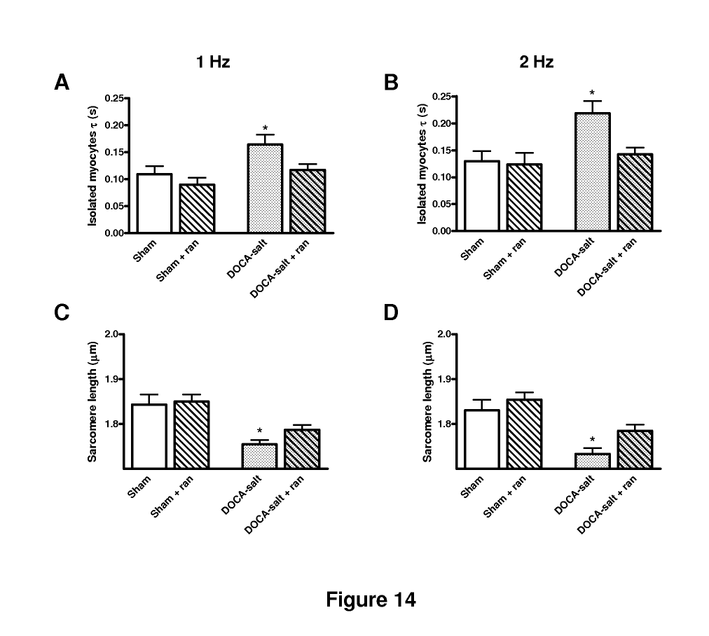

Ranolazine improved relaxation in DOCA-salt cardiomyocytes. Silberman et al. showed that impaired relaxation of cardiomyocytes was the main contributor to diastolic dysfunction in the DOCA-salt hypertensive model, finding no increase in cardiac fibrosis or inflammation.²⁰ To confirm ranolazine was working directly on DOCA-salt cardiomyocytes to improve relaxation, freshly isolated ventricular cardiomyocytes were stimulated at 0.5, 1, and 2 Hz at 37° C. and assessed. DOCA-salt cardiomyocytes had preserved contractile function, as previously reported²⁰ (FIGS. 8A and 8B). Additionally, treatment with ranolazine did not affect contraction in sham and DOCA-salt mice. In contrast, relaxation τ was significantly impaired in DOCA-salt mice and improved to normal levels with ranolazine treatment (DOCA-salt 0.18±0.02, DOCA-salt+ranolazine 0.13±0.01, Sham 0.11±0.01, Sham+ranolazine±0.09±0.02 s, p=0.0004; FIG. 8C). The baseline sarcomere length of cardiac myocytes was significantly reduced in DOCA-salt mice compared to sham mice (FIG. 8D). Treatment with ranolazine improved resting sarcomere length in the DOCA-salt mice to levels similar to sham, and ranolazine did not affect sham sarcomere length (DOCA-salt 1.75±0.01, DOCA-salt+ranolazine 1.81±0.01, sham 1.85±0.02, sham+ranolazine 1.86±0.02 μm, p<0.0001). In the absence of changes in resting Ca²⁺ (see below), this indicated that myofilament response to Ca²⁺ may be altered in diastolic dysfunction. Comparable effects were seen at 1 and 2 Hz stimulation rates and physiological temperature (FIG. 14). The effect of ranolazine in these experiments was reversible upon washout out (data not shown).

Diastolic Dysfunction was Independent of Intracellular Calcium Cycling.

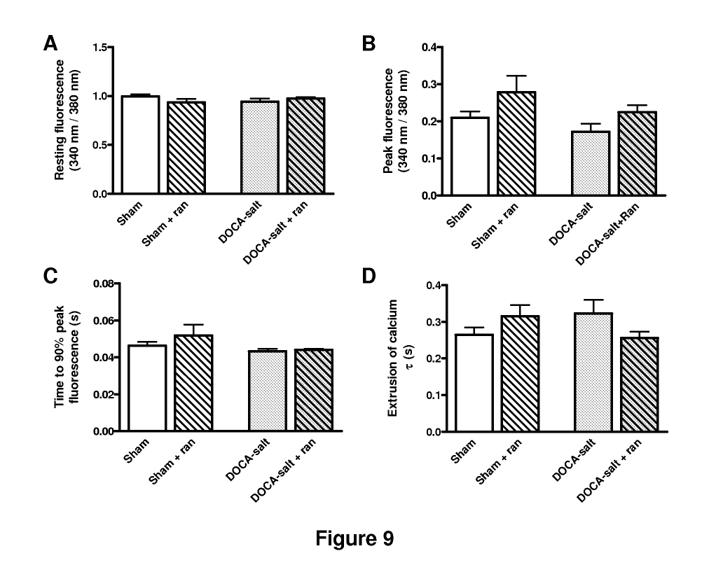

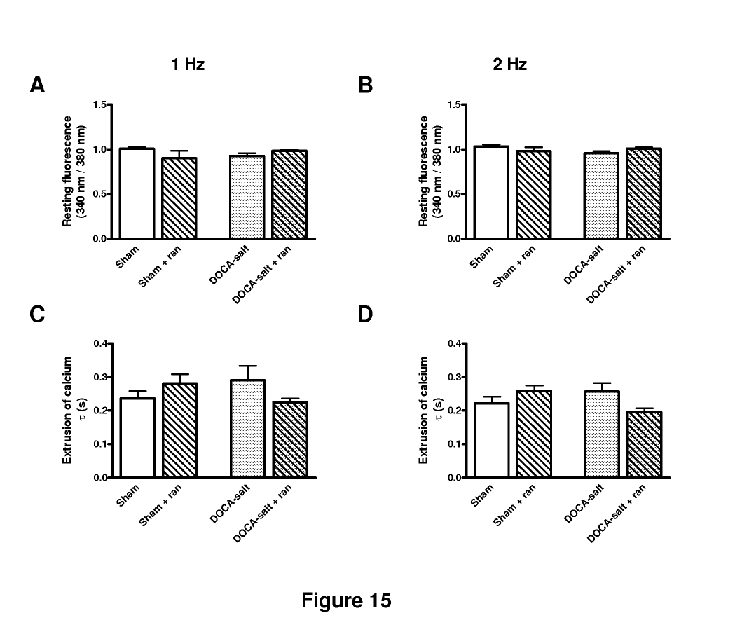

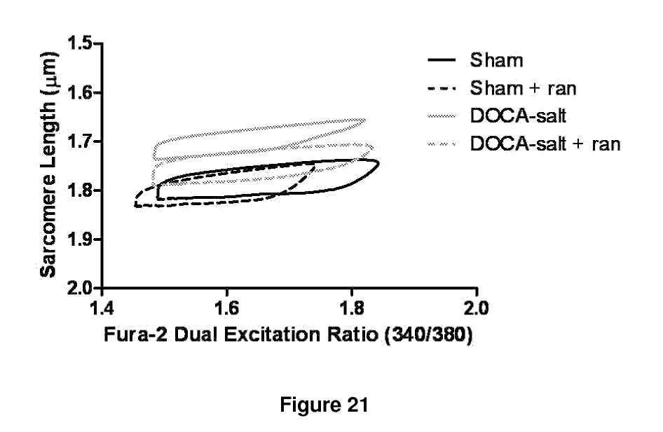

To elucidate the mechanism underlying the impaired diastolic function in hypertensive mice, Ca²⁺ transients were measured in freshly isolated ventricular myocytes. Ca²⁺ transients did not differ significantly between sham and DOCA-salt mice, and the addition of 10 μM ranolazine did not affect either group (FIG. 9). Baseline intracellular Ca²⁺ was similar in all groups (FIG. 9A). Additionally, peak intracellular Ca²⁺ and the rate of Ca²⁺ release was similar between all four groups (FIGS. 9B and 9C). Surprisingly, there was no difference among the rates of intracellular Ca²⁺ egress among the four groups measured as the time constant τ (FIG. 9D). This held true at pacing rates of 1 and 2 Hz as well (FIG. 15). We cannot rule out regional variations in myocyte properties may obscure changes in Ca²⁺ handling, however. In FIG. 21, we show phase-plane plots that of the averaged Fura-2 fluorescence dual excitation ratio (340/380) vs. sarcomere length during entire contraction-relaxation cycle in sham, DOCA-salt, and ranolazine treated groups.

Late I_{Na} was not elevated in DOCA-Salt Mice

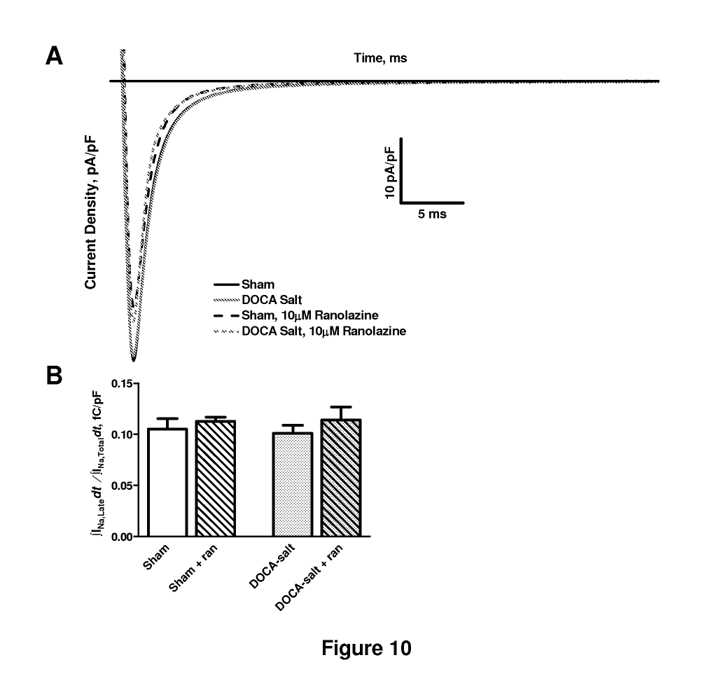

Oxidative stress is known to induce late I_{Na} that can be blocked by the anti-anginal drug, ranolazine, and the DOCA-salt model is associated with increased cardiac oxidative stress.^{20,24} Nevertheless, voltage-clamp studies show no increase in late I_{Na} in DOCA-salt cardiomyocytes compared to sham (Sham 0.105±0.01, Sham+ranolazine 0.113±0.004, DOCA-salt 0.101±0.008, DOCA-salt+ranolazine 0.114±0.013, p=NS) (FIG. 10A). Integrated late I_{Na} was measured starting at 5% of peak current and ending 40 ms after depolarization. DOCA-salt myocytes accumulated a similar amount of charge as sham. Extracellular addition of 10 μmol/L ranolazine did not affect the late accumulated charge in DOCA-salt cardiomyocytes, which was similar to that seen for sham and sham treated myocytes (FIG. 10B).

Skinned Fibers from DOCA-Salt Treated Hearts Demonstrated Increased Response to Ca2+ and Depressed Cross-Bridge Kinetics.

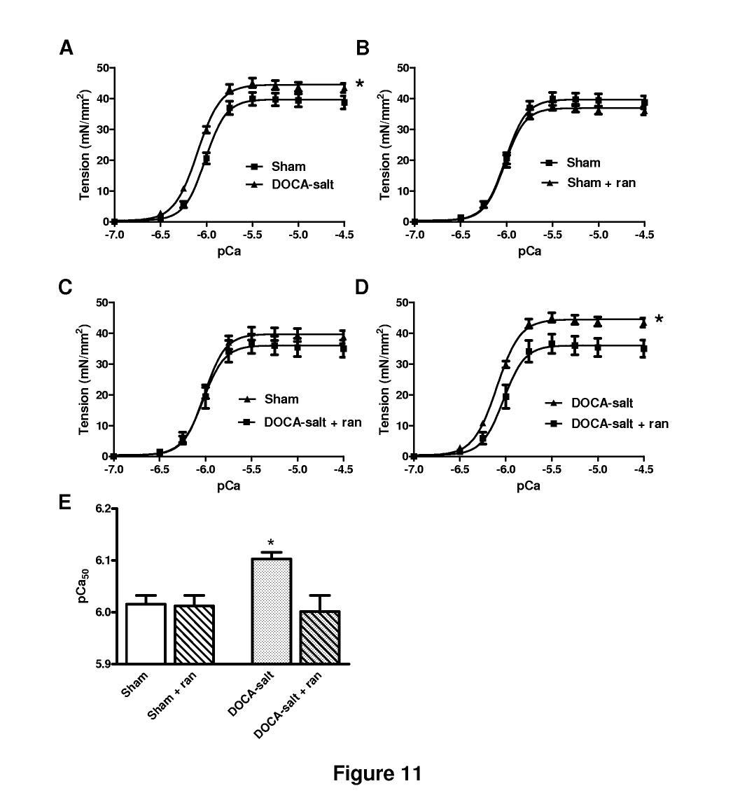

To examine directly myofilament function, the Ca-sensitivity of steady-state isometric tension development was measured in a first series of experiments with skinned fiber bundles from sham and DOCA-salt heart preparations. With DOCA-salt treatment, the mean steady-state isometric tension demonstrated an increase in maximum tension and a small but significant leftward shift of the pCa-tension relation (FIG. 11, left panel). Myofilament Ca²⁺ sensitivity, indexed by pCa₅₀, was significantly (p<0.02) greater in DOCA-salt than in sham myofilaments (FIG. 11). As indicated in FIG. 11, ranolazine treatment reduced maximum tension and returned the DOCA-salt myofilament sensitivity to levels similar to sham fibers with little effect on fiber bundles from sham hearts. The changes in Ca²⁺ sensitivity are statistically significant and in a direction that explain the decreased diastolic function. Nevertheless, we cannot completely rule out other possible mechanisms for decreased diastolic relaxation. These results imply that resting tension in Ca²⁺-free conditions should be increased, although this was not tested here.

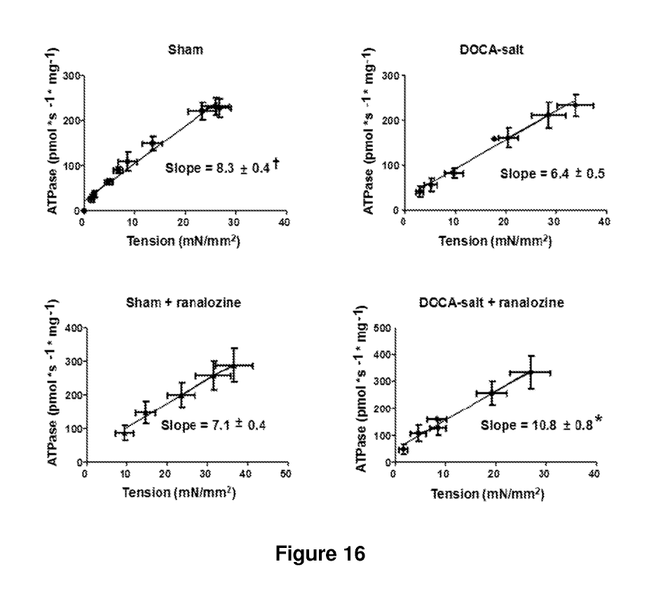

In order to assess whether the fiber bundles from DOCA-salt treated hearts demonstrated altered cross-bridge kinetics, we measured tension development and simultaneously the rate of ATP hydrolysis (FIG. 16). As summarized in Table 6, there was a significant increase in maximum tension of fiber bundles from DOCA-salt treated hearts compared to shams.

TABLE 6
Table 1C. Effect of Ranolazine on tension and ATPase
rate of skinned fiber bundles

| | | Sham + | | DOCA-salt + |
|---|---|---|---|---|
| | Sham | ranolazine | DOCA-salt | ranolazine |
| Maximum | 26.84 ± 0.35*^{#} | 36.80 ± 0.16 | 33.46 ± 0.38 | 27.19 ± 0.54 |
| Tension | | | | |
| (mN/mm²) | | | | |
| Maximum | 233.2 ± 2.9†^{#} | 302.0 ± 9.0* | 234.6 ± 3.7 | 326.9 ± 8.7* |
| ATPase | | | | |
| (pmol * | | | | |
| s-1 * | | | | |
| mg-1) | | | | |
| Tension Cost | 8.3 ± 0.4* | 7.1 ± 0.4 | 6.4 ± 0.5 | 10.8 ± 0.8* |
| ΔATPase/ | | | | |
| ΔTension | | | | |
| Passive | 0.45 ± 0.26 | 0.55 ± 0.33 | 0.20 ± 0.32 | 0.68 ± 0.40 |
| Tension | | | | |
| (mN/mm²) | | | | |
| Ktr | 10.9 ± 0.7 | 11.2 ± 0.5 | 11.2 ± 0.9 | 11.0 ± 1.0 |

Data are means ± standard error.
Ktr, the rate constant for force redevelopment.
N = 5-7,
*p < 0.05 vs. DOCA-salt.
†p < 0.05 vs. DOCA-salt + ranolazine.
^{#}p < 0.05 vs. sham + ranolazine.

Treatment of the fiber bundles from DOCA-salt treated hearts with ranolazine restored tension to the control levels. Nevertheless, measurement of ATPase rates during the development of steady-state tension showed responses different from the tension measurements. There was no significant difference of maximum ATPase rate between fibers from sham controls compared to fibers from DOCA-salt treated hearts. Treatment of the fibers from DOCA-salt hearts with ranolazine induced a significant increase in ATPase rate.

FIG. 16 shows the ATPase rate plotted as a function of tension. The slope of this relation, which provides a measure of tension cost, was significantly depressed in fibers from DOCA-salt treated hearts compared to shams, implying a slowing of myofilament exit from the cross-bridge cycle (Table 6).²⁵ Moreover, although ranolazine had no significant effect on the skinned fibers from shams, treatment of the DOCA-salt fibers with ranolazine significantly increased the maximum ATPase rate over that of the fibers from DOCA-salt treated hearts. We also determined Ktr, which is a measure of the rate of re-entry of cross-bridges into force generating states. No change in Ktr was observed under any of the conditions studied. In a separate set of experiments, we measured myofilament calcium sensitivity of fibers from hearts of DOCA-salt mice and DOCA-salt mice chronically treated with ranolazine and confirmed that ranolazine treatment significantly reduces maximal tension in response to Ca²⁺ in vivo. While undetected changes in Na⁺ and Ca²⁺ currents under stretch cannot be completely eliminated as a cause of diastolic dysfunction, because these experiments were carried out in skinned fibers, our data suggest that a major cause is alterations in the myofilaments that are ameliorated by ranolazine.

Myofilament Protein Phosphorylation and Oxidation Analysis

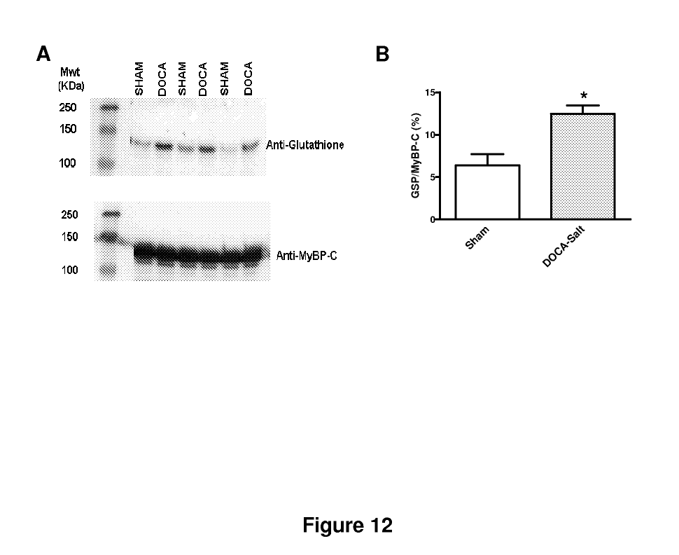

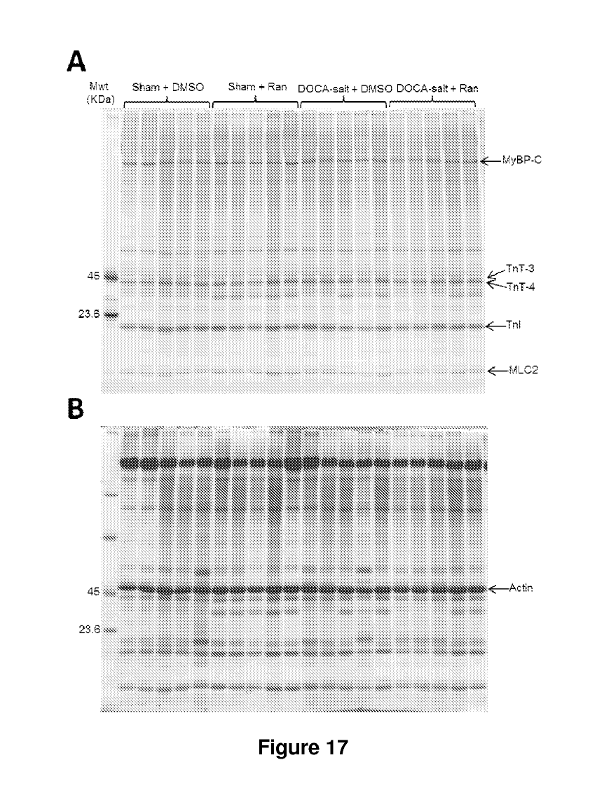

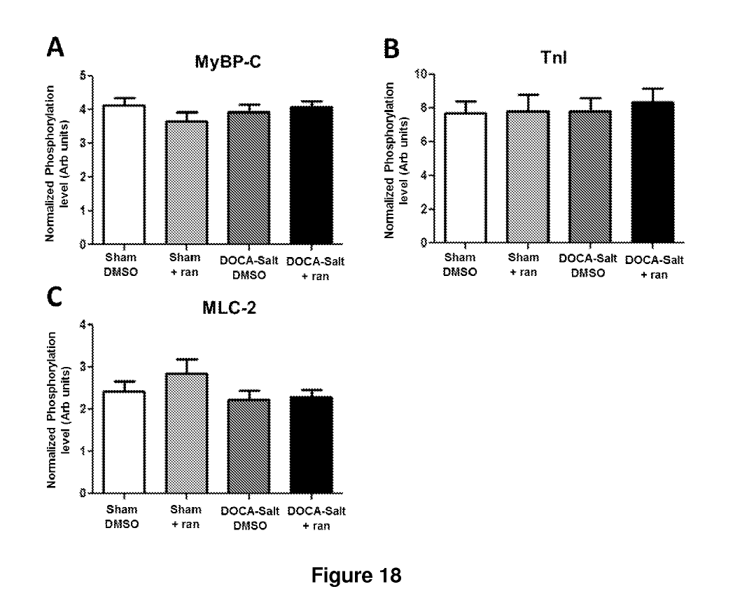

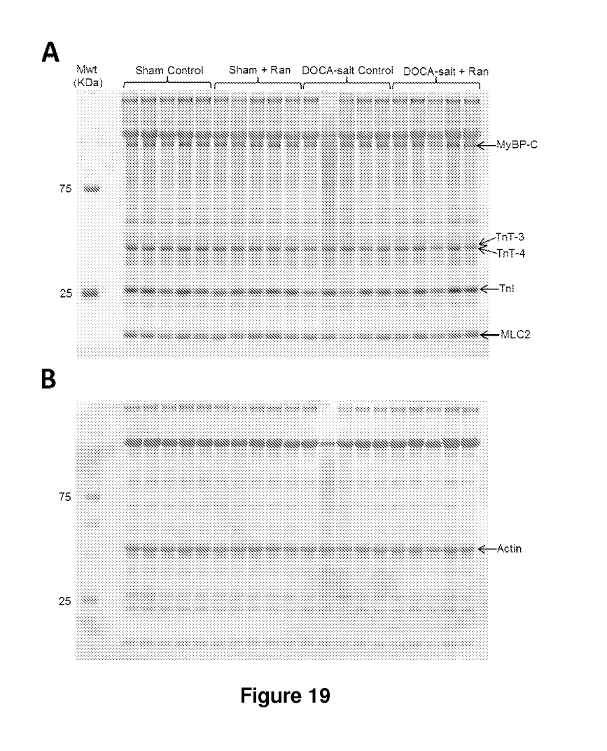

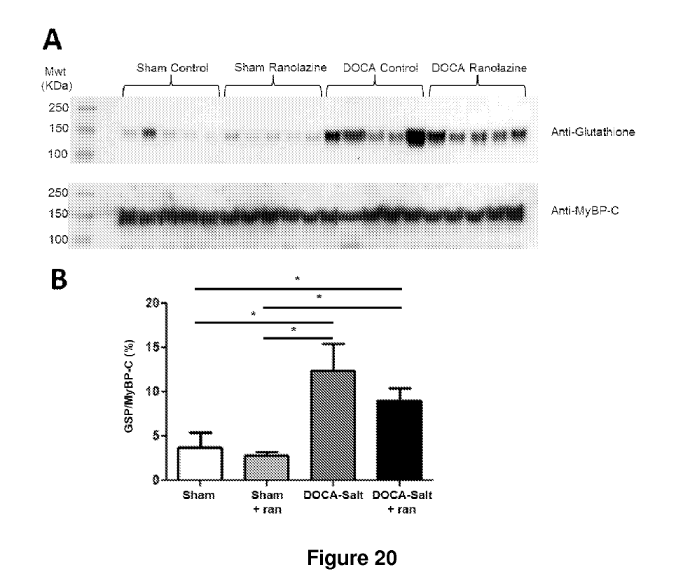

To understand the mechanism whereby hypertension generated changes in myofilament contractile properties, we performed an analysis of myofilament protein abundance and post-translational modifications. Representative ProQ and Coomassie gels can be seen in FIGS. 17 and 19. There were no changes in the abundances of myofilaments or the levels of phosphorylation of major myofilament proteins such as myosin binding protein C, troponin I, and myosin light chain 2 (FIG. 18). Similar findings were noted with chronic ranolazine administration. Additionally, there was an increase in S-glutathionylation of myosin binding protein C in DOCA-salt mice (FIGS. 12A and 12B). We determined S-glutathionylation levels in DOCA-salt and sham hearts from mice after or without chronic administration of ranolazine, which confirmed our previous results (FIG. 20). These data indicate that there are DOCA-salt induced changes in the state of critical myofilament proteins, which may explain the changes in myofilament activity.

### Example 8

This example demonstrates a randomized, double-blinded, placebo-controlled and single center clinical trial of patients presenting with acute decompensated hearl failure randomized in a one to one ratio to treatment with ranolazine (initiated intravenously and followed by oral ranolazine extended-release) or matched placebo within 24 hours of initial evaluation given in addition to standard therapy.

In order to test the hypothesis that ranolazine will improve functional capacity and both systolic and diastolic echocardiographic parameters in patients presenting with heart failure compared to placebo, the following clinical trial was designed. The study aims to evaluate the efficacy of ranolazine in acute decompensated heart failure to increase functional capacity in patients and to evaluate the efficacy of ranolazine in acute decompensated heart failure to improve both systolic and diastolic parameters of myocardial function.

The study will be a randomized, double-blinded, placebo-controlled and single center clinical trial of patients presenting with acute decompensated heart failure randomized in a one to one ratio to treatment with ranolazine (initiated intravenously and followed by oral ranolazine extended-release) or matched placebo within 24 hours of initial evaluation given in addition to standard therapy. Endpoints will include clinical and echocardiographic parameters at discharge. We will enroll and consent patients in the emergency departments of the University of Illinois Medical Center at Chicago, Ill. (UICMC) and the Jesse Brown VA Medical Center (JB VAMC). Patients may also be enrolled from the hospital wards if they are available to receive the study drug within 24 hours of initial evaluation.

The following is a listing of criteria for including patients in the study:
1. Age greater than or equal to 18 years
2. Admission for acute decompensated heart failure as evidenced by clinical parameters and NT-proBNP>300 pg/mL⁴¹ within 24 hours
3. Patient at UICMC or JB VAMC
4. Ambulatory without assist device
5. Able to provide informed consent

The following is a listing of criteria for excluding patients in the study:
1. Prior or current use of ranolazine
2. Use of strong CYP3A inhibitors including ketoconazole, itraconazole, clarithromycin, nefazodone, nelfinavir, ritonavir, indinavir and saquinavir
3. Use of P-glycoprotein inhibitors including cyclosporine
4. Use of CYP3A and P-glycoprotein inducers including rifampin, rifabutin, rifapentin, phenobarbital, phenyloin, carbamazepine and St. John's Wort
5. Use of drugs transported by P-glycoprotein or metabolized by CYP2D6 including digoxin and all tricyclic antidepressants
6. Current pregnancy or active nursing
7. QTc interval>500 msec or use of drugs known to prolong QTc interval
8. Complete Bundle Branch Block, QRS duration>120 rnsec
9. Rhythm other than Normal Sinus at Enrollment including any Paced Rhythms
10. Moderate to Severe Aortic or Mitral Valve Disease
11. Hemodynamically Significant Left Ventricular Outflow Tract Obstruction
12. Prosthetic Valve
13. Acute Coronary Syndrome (ACS) or ACS within 6 weeks
14. Cardiogenic Shock
15. Active Use of Intravenous Vasodilators, Vasopressors or Inotropes
16. History of Heart Transplant or Left Ventricular Assist Device
17. Uncontrolled Hypertension (Blood Pressure>180/100 at rest) on Medications
18. Pulmonary Arterial Hypertension (Group 1)⁴²
19. Hemodialysis, Peritoneal Dialysis or Creatinine>2.0 mg/dL
20. Cirrhosis or Clinically Significant Hepatic Impairment
21. Active Infection including Bacteremia
22. Major Trauma or Surgery within 6 weeks
23. Malignant Neoplastic Disease
24. Collagen Vascular Disease
25. Illicit drug use or alcohol abuse within 6 weeks
26. Concomitant use of investigational drug within 6 weeks
27. Systemic steroid use within 6 weeks
28. Severe disease limiting life expectancy to approximately less than 1 year

Subject Recruitment

Patients will be identified from review of admission logs on a daily basis to both the emergency department and medical wards of the involved institutions. Pre enrollment screening will involve reviewing history and physicals and laboratory data to verify that the admitting diagnosis for congestive heart failure is supported by clinical assessment and NT-proBNP>300 pg/mL. If a patient is eligible after inclusion and exclusion criteria are evaluated, permission will be obtained from the primary physician prior to approaching the patient to explain the study and drug risks and to obtain signed informed consent. Patients may decline to participate or withdraw at any time with no change in clinical care and with assurance of strict confidentiality. Consent will not be required for eligibility screening.

After enrollment, baseline characteristics will be recorded including demographics, medical history, cardiac history, cardiovascular medications, laboratory data and echocardiographic parameters if a transthoracic echocardiogram is available within 3 months prior to presentation.

Randomization and Blinding

Patients will be randomized to treatment with ranolazine or placebo in a one to one ratio by a central computerized system using permuted-block randomization without stratification. The study will be conducted in a double-blinded placebo-controlled manner with placebo infusion solution and capsules identical to ranolazine preparations. Drug bags and bottles will be labeled uniquely for identification and supplied by a central pharmacy. The code will be broken only in exceptional circumstances when required for clinical management of patients as determined by primary physician and research team.

Intervention

All patients will receive standard therapy for heart failure as determined by their primary physicians. In addition, the ranolazine group will receive 200 mg intravenously over one hour followed by an infusion of 80 mg/hr for 48 hours which will be reduced to 40 mg/hr if they are taking a moderate inhibitor of CYP3A including verapamil, diltiazem and erythromycin. After completion of the infusion patients will receive ranolazine extended-release tablets at an oral dose of 1000 mg twice daily. Patients on a moderate CYP3A inhibitor will receive 500 mg twice daily. Patients will take the drug until discharge from the hospital at which point participation will be complete. Safety of ranolazine at these suggested doses is well documented by the MARISA, CARISA, ERICA and MERLIN-TIMI 36 investigator groups.¹⁹⁻²² The dose reductions suggested for patients on moderate inhibitors of CYP3A are manufacturer recommendations, which were updated in September 2010.⁴³ The non-treatment group will receive matching placebo for intravenous and oral administration.

End Points

The primary efficacy outcome of the trial will be performance on the six-minute walk test (6MWT) strictly conducted according to guidelines set by the American Thoracic Society at the time of discharge.⁴⁴ The 6MWT is a safe and validated measure of functional status in patients with congestive heart failure and correlates with survival, repeat hospitalization for heart failure and New York Heart Association class.⁴⁵ In a study of mild to moderate heart failure, patients able to walk greater than 450 meters compared to those who could only walk 300 meters or less had improved survival and distance walked proved to be an independent predictor of morbidity and mortality.⁴⁶

Secondary clinical end points will include the number of hours to transition from intravenous diuretics to oral diuretics, length of stay, cardiovascular death during index hospitalization and response to the Minnesota Living with Heart Failure Questionnaire (MLHFQ) at discharge. The MLHFQ is a validated measure of patients' own perception of quality of life and functional capacity when living with heart failure.^{47,48}

Echocardiographic parameters will also comprise secondary outcome measures and will be recorded by a physician board certified in echocardiography and blinded to clinical information. Both systolic and diastolic measures will be recorded including:
1. left ventricular ejection fraction (LVEF)
2. peak early (E) and peak late (A) diastolic mitral inflow velocities
3. E/A ratio
4. E deceleration time (DT)
5. tissue doppler, peak early (E′) and peak late (A′) mitral annular diastolic velocities
6. E′/A′ ratio
7. E/E′ ratio
8. isovolumic contraction time (ICT)
9. isovolumic relaxation time (IRT)
10. ejection time (ET)
11. myocardial performance index (MPI)

The MPI or Tei index, incorporates both systolic and diastolic parameters to measure overall left ventricular function and is easy to calculate and reproduce.⁴⁹ The MPI also correlates well with invasive measurements of systolic and diastolic function.^{50,51} We will measure the MPI as described by Figueredo et al.³⁸ We will also note if a patient has HFrEF as defined by LVEF 50% or HFpEF as defined by the European Society of Cardiology.⁵²

Statistical Analysis

A power analysis, assuming a 10 percent loss rate due to difficulty retaining patients, indicates that with a two-tailed alpha level of 0.05 and a test power of 0.90, the sample size required will be 11 patients in each group. This calculation is based on the intent to show a 150-meter difference in 6MWT between the two groups and draws information from an observational study of the difference in 6MWT by NYHA class.⁴⁶

Ranolazine and placebo groups will be compared, and analysis will be by intention to treat. It is anticipated that subjects treated with ranolazine will display increased walking distances on the 6MWT. Baseline data will be expressed as mean±SD for continuous variables and frequencies for categorical variables. Differences in baseline characteristics between the groups including 6MWT distances will be examined by use of Fisher exact and Mann-Whitney tests for categorical and continuous variables respectively. A value of p<0.05 will be considered statistically significant, suggesting that the null hypothesis of no difference between the groups should be rejected. Differences in time to secondary endpoint distributions will be analyzed using a proportional hazards regression, estimates of the median time to the endpoint and the proportion endpoint free.

Cox proportional hazards regression will be used to derive the hazard ratio for the study endpoints comparing ranolazine to placebo groups after adjustment for variables in which the distribution is not balanced between the two randomized groups. The modeling will take into consideration differences in demographic variables and cardiovascular medication use in addition to other baseline characteristics recorded at enrollment. In order to select variables to be included in the model, we will conservatively consider those variables with a different distribution between the two groups at a p<0.20. A stepwise backward procedure will be used, excluding variables above a value of p. 0.05 for the null hypothesis that the hazard ratio contributed by that variable is equal to 1.0. Results will be compared to stepwise forward procedures. The possibility of multicolinearity will be evaluated. Linear and non-linear terms will be considered. Discrimination of the model will be evaluated by an overall C index, validated by bootstrap methods and fit by examining the relationship between predicted and observed endpoints over a range of predicted events. The proportional hazards assumption will be checked by various methods including examination of log-log plots, testing Schoenfeld residuals and inclusion of terms representing time-dependent intPractions hetwPPn each of thP indPpPnriPnt variables and time. Results will be reported as the hazard ratio and its associated 95% confidence interval. The significance of this hazard ratio will be assessed by the likelihood ratio test. Data analysis will be done in collaboration with the University of Illinois Center for Clinical and Translational Science (CCTS). The statistical package used will be SAS.

Risk to Human Subjects

Human Subjects Involvement and Characteristics

Subjects included in the trial will be those presenting with acute decompensated heart failure. Exclusion criteria are not based upon sex or race except for pregnant women and nursing mothers. No vulnerable populations will be enrolled including the homeless and prisoners.

Sources of Materials

Material to be collected includes demographic data, echocardiographic data, medical history, inpatient clinical data, laboratory work and measurement of performance on the 6MWT. Testing done outside current clinical practice guidelines for heart failure include echocardiography at discharge and the 6MWT. No biological specimens will be collected.

Potential Risks

The study does not propose a substantial change to the standard of care for heart failure. Research risk revolves around reported side effects seen with ranolazine and potential effects of placebo administration common to this type of study. Patients, secondary to dizziness, nausea, asthenia, constipation and headache, occasionally discontinue Ranolazine. Serious side effects are rare.⁴³ Although ranolazine has a positive linear relationship with the QTc interval, the doses proposed in this study and our strict exclusion criteria, minimize the risk of adverse events. Furthermore, the MERLIN [symbol] TIMI 36 investigators found no proarrhythmic risk with ranolazine at clinically relevant doses.²² The alternative to the study is simply standard care for heart failure. Women who become pregnant during the study will be withdrawn immediately. Blinding will be broken in this instance and the patient will be referred to an obstetrician. There are no anticipated psychological, social or legal risks to patients for participating in the study to the best of our knowledge.

Recruitment and Informed Consent

Patients must be willing and able to give signed informed consent to enter the study. Patients will be approached only after their physicians have approved of enrollment. Research team personnel will obtain consent after patients have the opportunity to review the informed consent document, have the purpose and procedures explained to them and have an opportunity to ask questions. All patients will sign an informed consent form approved by the U1CMC and JB VAMC combined Internal Review Board (IRB) prior to randomization. The form will include sections on the purpose, procedures, risks and inconveniences, potential benefits, alternatives, the nature of voluntary participation and withdrawal, provisions for notification of novel findings, comments on confidentiality and protected health information and study contact persons. No compensation will be provided to patients for participation in this study.

Protection Against Risk

After acquisition all data will be stored in a computerized database without subject identifiers. Patients will be assigned a unique study number at randomization. All adverse events will be reported in writing at the time of their identification. An adverse event will be defined as any undesirable clinical experience occurring to the patient during the study, whether or not it is thought to be attributable to the intervention. An adverse event thought to be the result of the intervention will result in termination of the intervention without alteration of standard care.

Potential Benefit of Research

Based on prior research, it is possible that ranolazine may offer benefit in acute heart failure, especially in light of the fact that it has minimal effect on heart rate and blood pressure. Ranolazine, by improving both systolic and diastolic cardiac performance, may be able to improve functional capacity in patients with acute heart failure while shortening hospital stays and improving the therapeutic potential of standard therapy. For patients with recurrent and lengthy hospital stays and declining functional status due to heart failure, this research may offer an additional therapeutic option to improve quality of life.

Importance of the Knowledge Gained

If ranolazine does indeed improve 6MWT performance as well as other secondary measures, prior animal and human studies reporting improved cardiac indices with the drug will be validated. The knowledge gained from the trial will also indirectly assess the safety of ranolazine in acute heart failure. These two potential findings will support the use of ranolazine as adjunctive therapy in acute heart failure and will prompt further testing of the drug to determine long-term mortality and morbidity benefit of the drug in the heart failure population.

Safety and Monitoring Data Accuracy and Integrity

The study will be conducted in accordance with the International Committee on Harmonization (ICH) Good Clinical Practice guidelines and appropriate Federal regulations. All data records will be reviewed by the principal investigator for missing data prior to entry into the computerized database. Every effort will be made to obtain missing data at that time. Analysis will be conducted with and without imputation of missing data.

Data Monitoring Board (DMB)

An independent DMB will monitor the results at no less than two-month intervals to recommend to the IRB whether or not to continue the study. The DMB will be comprised of directors of Internal Medicine at both UICMC and JB VAMC with Hip addition of an independpnt hinqtAtistiriAn At the (TIN to review the conduct of the trial, all adverse events and data collected from the trial including abnormal laboratory values and case report forms. All data will be presented without subject identifiers to maintain confidentiality. Adverse events will be tabulated according to treatment group and duration. All serious adverse events designated by the investigators will be reviewed at the time of reporting. Serious adverse events will be defined by the ICH. Adverse events will be compared by calculating the relative risks between groups. Unanticipated adverse events will be investigated and reported promptly to the IRB. Once unanticipated adverse events are reported, the DMB will investigate to determine if the event represents an unreasonable risk to the subject so as to terminate the study.

Target Dates for Start and Completion

The projected start date would be within two months of approval from the IRB and the anticipated study period is approximately four to six months. The heart failure teams at UICMC alone follow a total census of approximately 400 patients as both inpatient and outpatient, so there should be no issue recruiting the appropriate sample size for this study.

The following listing provides citations to references cited in Example 8:
1. Lloyd-Jones et al., Circulation 2002; 106(24):3068-3072.
2. McMurray et al., Eur Heart J2002; 23(23):1867-1876.
4. Cowie et al., Heart 2000; 83(5):505-510.
5. Owan et al., N Engl J Med 2006; 355(3):251-259.
6. Bhatia et al., N Engl J Med 2006; 355(3):260-269.
7. Effect of enalapril on survival in patients with reduced left ventricular ejection fractions and congestive heart failure. The SOLVD Investigators. N Engl J Med 1991; 325(5):293-302.
8. Flather et al., Lancet 2000; 355(9215):1575-1581.
9. Poole-Wilson et al., Lancet 2003; 362(9377):7-13.
10. Yusuf et al., Lancet 2003; 362(9386):777-781.
11. Cleland et al., Eur Heart J 2006; 27 (19):2338-2345.
12. Massie et al., N Engl J Med 2008; 359(23):2456-2467.
13. Shah et al., J Card Fail 2010; 16(3):260-267.
14. Stewart et al., Eur J Heart Fail 2002; 4(3):361-371.
15. Murphy et al., Heart 2004; 90(10):1129-1136.
16. Belardinelli et al., Heart 2006; 92 Suppl 4:iv6-iv14.
17. Maier et al., J Cardiovasc Pharmacol 2009; 54(4):279-286.
18. Stone et al., Cardiol Clin 2008; 26(4):603-614.
19. Chaitman et al., J Am Coll Cardiol 2004; 43(8):1375-1382.
20. Chaitman et al., JAMA 2004; 291(3):309-316.
21. Stone et al., J Am Coll Cardiol 2006; 48(3):566-575.
22. Morrow et al., JAMA 2007; 297(16):1775-1783.
23. Maltsev et al., Eur J Heart Fail 2007; 9(3):219-227.
24. Valdivia et al., J Mol Cell Cardiol 2005; 38(3):475-483.
25. Undrovinas et al., J Cardiovasc Electrophysiol 2006; 17 Suppl 1:S169-S177.
26. Sossalla et al., J Mal Cell Cardiol 2008; 45(1):32-43.
27. Fraser et al., J Mol Cell Cardiol 2006; 41(6):1031-1038.
28. Wu et al., J Pharmacol Exp Ther 2009; 330(2):550-557.
29. Maruyama et al., J Pharm Pharmacol 2000; 52(6):709-715.
30. Gralinski et al., Cardiovasc Res 1994; 28(8):1231-1237.
31. Song et al., J Pharmacol Exp Ther 2006; 318(4214-222.
32. Matsumura et al., Jpn J Pharmacol 1998; 77(1):31-39.
33. Sossalla et al., Basic Res Cardiol 2010; 106(2): 263-272
34. Harvey et al., Circulation 2010. Ref Type: Abstract
35. Sabbah et al., J Card Fail 2002; 8(6):416-422.
36. Chandler et al., Circ Res 2002; 91(4):278-280.
37. Rastogi et al., Am J Physiol Heart Circ Physiol 2008; 295(5):H2149-H2155.
38. Figueredo et al., J Cardiovasc Pharmacol Ther 2010; 16(2): 168-172.
39. Moss et al., Cardiovasc Electrophysiol 2008; 19(12):1289-1293.
40. Hayashida et al., Cardiovasc Drugs Ther 1994; 8(5):741-747.
41. Januzzi et al., Eur Heart 2006; 27(3):330-337.
42. Simonneau et al., J Am Coll Cardiol 2009; 54(1 Suppl):S43-S54.
43. Gilead Sciences Inc. Ranolazine Prescribing Information. 2010. Ref Type: Online Source
44. ATS statement: guidelines for the six-minute walk test. Am J Respir Crit. Care Med 2002; 166(4111-4117.
45. Bittner et al., JAMA 1993; 270(14):1702-1707.
46. Rostagno et al., Eur J Heart Fail 2003; 5(3):247-252.
47. Rector et al., Am Heart j 1992; 124(4):1017-1025.
48. Garin et al., Qual Life Res 2009; 18(1):71-85.
49. Tei et al., J Cardiol 1995; 26(6):357-366.
50. Tei et al., J Am Soc Echocardiogr 1997; 10(2):169-178.
51. Su et al., Echocardiography 2007; 24(3):252-257.
52. Paulus et al., Eur Heart J2007; 28(20):2539-2550.

### REFERENCES

The following represents a reference list numbered according to the citation numbering used in Examples 1-6:
1. Owan T E, Hodge D O, Herges R M, Jacobsen S J, Roger V L, Redfield M M. Trends in prevalence and outcome of heart failure with preserved ejection fraction. N Engl J Med. 2006; 355(3):251-259.
2. Redfield M M, Jacobsen S J, Burnett J C, Jr., Mahoney D W, Bailey K R, Rodeheffer R J. Burden of systolic and diastolic ventricular dysfunction in the community: appreciating the scope of the heart failure epidemic. JAMA. 2003; 289(2):194-202.
3. Bursi F, Weston S A, Redfield M M, Jacobsen S J, Pakhomov S, Nkomo V T, Meverden R A, Roger V L. Systolic and diastolic heart failure in the community. JAMA. 2006; 296(18):2209-2216.
4. Schocken D D, Benjamin E J, Fonarow G C, Krumholz H M, Levy D, Mensah G A, Narula J, Shor E S, Young J B, Hong Y. Prevention of heart failure: a scientific statement from the American Heart Association Councils on Epidemiology and Prevention, Clinical Cardiology, Cardiovascular Nursing, and High Blood Pressure Research; Quality of Care and Outcomes Research Interdisciplinary Working Group; and Functional Genomics and Translational Biology Interdisciplinary Working Group. Circulation. 2008; 117(19):2544-2565.
5. Ouzounian M, Lee D S, Liu P P. Diastolic heart failure: mechanisms and controversies. Nat Clin Pract Cardiovasc Med. 2008; 5(7):375-386.
6. Ahmed A, Rich M W, Fleg J L, Zile M R, Young J B, Kitzman D W, Love T E, Aronow W S, Adams K F, Jr., Gheorghiade M. Effects of digoxin on morbidity and mortality in diastolic heart failure: the ancillary digitalis investigation group trial. Circulation. 2006; 114(5):397-403.
7. Hernandez A F, Hammill B G, O'Connor C M, Schulman K A, Curtis L H, Fonarow G C. Clinical effectiveness of beta-blockers in heart failure: findings from the OPTIMIZE-HF (Organized Program to Initiate Lifesaving Treatment in Hospitalized Patients with Heart Failure) Registry. J Am Coll Cardiol. 2009; 53(2):184-192.
8. Massie B M, Carson P E, McMurray J J, Komajda M, McKelvie R, Zile M R, Anderson S, Donovan M, Iverson E, Staiger C, Ptaszynska A. Irbesartan in patients with heart failure and preserved ejection fraction. N Engl J. Med. 2008; 359(23):2456-2467.
9. Bers D M. Excitation-contraction coupling and cardiac contractile force. 2nd ed. Dordrecht; Boston: Kluwer Academic Publishers; 2001.
10. Ju Y K, Saint D A, Gage P W. Hypoxia increases persistent sodium current in rat ventricular myocytes. J Physiol. 1996; 497 (Pt 2):337-347.
11. Undrovinas A I, Fleidervish I A, Makielski J C. Inward sodium current at resting potentials in single cardiac myocytes induced by the ischemic metabolite lysophosphatidylcholine. Circ Res. 1992; 71(5):1231-1241.
12. Wu J, Corr P B. Palmitoyl carnitine modifies sodium currents and induces transient inward current in ventricular myocytes. Am J Physiol. 1994; 266(3 Pt 2):H1034-1046.
13. Ward C A, Giles W R. Ionic mechanism of the effects of hydrogen peroxide in rat ventricular myocytes. J Physiol. 1997; 500 (Pt 3):631-642.
14. Hale S L, Shryock J C, Belardinelli L, Sweeney M, Kloner R A. Late sodium current inhibition as a new cardioprotective approach. J Mol Cell Cardiol. 2008; 44(6):954-967.
15. Song Y, Shryock J C, Wagner S, Maier L S, Belardinelli L. Blocking late sodium current reduces hydrogen peroxide-induced arrhythmogenic activity and contractile dysfunction. J Pharmacol Exp Ther. 2006; 318(1):214-222.
16. Flagg T P, Cazorla O, Remedi M S, Haim T E, Tones M A, Bahinski A, Numann R E, Kovacs A, Schaffer J E, Nichols C G, Nerbonne J M. Ca²⁺-independent alterations in diastolic sarcomere length and relaxation kinetics in a mouse model of lipotoxic diabetic cardiomyopathy. Circ Res. 2009; 104(1):95-103.
17. Wolska B M, Kitada Y, Palmiter K A, Westfall M V, Johnson M D, Solaro R J. CGP-48506 increases contractility of ventricular myocytes and myofilaments by effects on actin-myosin reaction. Am J Physiol. 1996; 270(1 Pt 2):H24-32.
18. Pohlmann L, Kroger I, Vignier N, Schlossarek S, Kramer E, Coirault C, Sultan K R, El-Armouche A, Winegrad S, Eschenhagen T, Carrier L. Cardiac myosin-binding protein C is required for complete relaxation in intact myocytes. Circ Res. 2007; 101(9):928-938.
19. Davis J, Wen H, Edwards T, Metzger J M. Thin filament disinhibition by restrictive cardiomyopathy mutant R193H troponin I induces Ca²⁺-independent mechanical tone and acute myocyte remodeling. Circ Res. 2007; 100(10):1494-1502.
20. Silberman G A, Fan T H, Liu H, Jiao Z, Xiao H D, Lovelock J D, Boulden B M, Widder J, Fredd S, Bernstein K E, Wolska B M, Dikalov S, Harrison D G, Dudley S C, Jr. Uncoupled cardiac nitric oxide synthase mediates diastolic dysfunction. Circulation. 2010; 121(4):519-528.
21. Cocco G, Rousseau M F, Bouvy T, Cheron P, Williams G, Detry J M, Pouleur H. Effects of a new metabolic modulator, ranolazine, on exercise tolerance in angina pectoris patients treated with beta-blocker or diltiazem. J Cardiovasc Pharmacol. 1992; 20(1):131-138.
22. Pepine C J, Wolff A A. A controlled trial with a novel anti-ischemic agent, ranolazine, in chronic stable angina pectoris that is responsive to conventional antianginal agents. Ranolazine Study Group. Am J Cardiol. 1999; 84(1):46-50.
23. Patton C, Thompson S, Epel D. Some precautions in using chelators to buffer metals in biological solutions. Cell Calcium. 2004; 35(5):427-431.
24. Wolska B M, Vijayan K, Arteaga G M, Konhilas J P, Phillips R M, Kim R, Naya T, Leiden J M, Martin A F, de Tombe P P, Solaro R J. Expression of slow skeletal troponin I in adult transgenic mouse heart muscle reduces the force decline observed during acidic conditions. J. Physiol. 2001; 536(Pt 3):863-870.
25. Kasner M, Westermann D, Steendijk P, Gaub R, Wilkenshoff U, Weitmann K, Hoffmann W, Poller W, Schultheiss H P, Pauschinger M, Tschope C. Utility of Doppler echocardiography and tissue Doppler imaging in the estimation of diastolic function in heart failure with normal ejection fraction: a comparative Doppler-conductance catheterization study. Circulation. 2007; 116(6):637-647.
26. Maier L S. A novel mechanism for the treatment of angina, arrhythmias, and diastolic dysfunction: inhibition of late I_{(Na)} using ranolazine. J Cardiovasc Pharmacol. 2009; 54(4):279-286.
27. Antzelevitch C, Belardinelli L, Zygmunt A C, Burashnikov A, Di Diego J M, Fish J M, Cordeiro J M, Thomas G. Electrophysiological effects of ranolazine, a novel antianginal agent with antiarrhythmic properties. Circulation. 2004; 110(8):904-910.
28. Hiranandani N, Bupha-Intr T, Janssen P M. SERCA overexpression reduces hydroxyl radical injury in murine myocardium. Am J Physiol Heart Circ Physiol. 2006; 291(6):H3130-3135.
29. Kuster G M, Lancel S, Zhang J, Communal C, Trucillo M P, Lim C C, Pfister O, Weinberg E O, Cohen R A, Liao R, Siwik D A, Colucci W S. Redox-mediated reciprocal regulation of SERCA and Na⁺—Ca²⁺ exchanger contributes to sarcoplasmic reticulum Ca²⁺ depletion in cardiac myocytes. Free Radic Biol Med. 2010; 48(9):1182-1187.
30. Wagner S, Seidler T, Picht E, Maier L S, Kazanski V, Teucher N, Schillinger W, Pieske B, Isenberg G, Hasenfuss G, Kogler H. Na⁺—Ca²⁺ exchanger overexpression predisposes to reactive oxygen species-induced injury. Cardiovasc Res. 2003; 60(2):404-412.
31. MacFarlane N G, Miller D J. Effects of the reactive oxygen species hypochlorous acid and hydrogen peroxide on force production and calcium sensitivity of rat cardiac myofilaments. Pflugers Arch. 1994; 428(5-6):561-568.
32. Wang H, Kohr M J, Traynham C J, Wheeler D G, Janssen P M, Ziolo M T. Neuronal nitric oxide synthase signaling within cardiac myocytes targets phospholamban. Am J Physiol Cell Physiol. 2008; 294(6): C1566-1575.
33. Monasky M M, Varian K D, Davis J P, Janssen P M. Dissociation of force decline from calcium decline by preload in isolated rabbit myocardium. Pflugers Arch. 2008; 456(2):267-276.
34. LeWinter M M. Functional consequences of sarcomeric protein abnormalities in failing myocardium. Heart Fail Rev. 2005; 10(3):249-257.
35. Sossalla S, Wagner S, Rasenack E C, Ruff H, Weber S L, Schondube F A, Tirilomis T, Tenderich G, Hasenfuss G, Belardinelli L, Maier L S. Ranolazine improves diastolic dysfunction in isolated myocardium from failing human hearts—role of late sodium current and intracellular ion accumulation. J Mol Cell Cardiol. 2008; 45(1):32-43.
36. Hwang H, Arcidi J M, Jr., Hale S L, Simkhovich B Z, Belardinelli L, Dhalla A K, Shryock J C, Kloner R A. Ranolazine as a cardioplegia additive improves recovery of diastolic function in isolated rat hearts. Circulation. 2009; 120(11 Suppl):S16-21.
37. Gralinski M R, Black S C, Kilgore K S, Chou A Y, McCormack J G, Lucchesi B R. Cardioprotective effects of ranolazine (RS-43285) in the isolated perfused rabbit heart. Cardiovasc Res. 1994; 28(8): 1231-1237.
38. Maruyama K, Hara A, Hashizume H, Ushikubi F, Abiko Y. Ranolazine attenuates palmitoyl-L-carnitine-induced mechanical and metabolic derangement in the isolated, perfused rat heart. J Pharm Pharmacol. 2000; 52(6):709-715.
39. Undrovinas A I, Belardinelli L, Undrovinas N A, Sabbah H N. Ranolazine improves abnormal repolarization and contraction in left ventricular myocytes of dogs with heart failure by inhibiting late sodium current. J Cardiovasc Electrophysiol. 2006; 17 Suppl 1:S169-S177.
40. Sabbah H N, Chandler M P, Mishima T, Suzuki G, Chaudhry P, Nass O, Biesiadecki B J, Blackburn B, Wolff A, Stanley W C. Ranolazine, a partial fatty acid oxidation (pFOX) inhibitor, improves left ventricular function in dogs with chronic heart failure. J Card Fail. 2002; 8(6):416-422.
41. Rastogi S, Sharov V G, Mishra S, Gupta R C, Blackburn B, Belardinelli L, Stanley W C, Sabbah H N. Ranolazine combined with enalapril or metoprolol prevents progressive LV dysfunction and remodeling in dogs with moderate heart failure. Am J Physiol Heart Circ Physiol. 2008; 295(5):H2149-2155.
42. Hayashida W, van Eyll C, Rousseau M F, Pouleur H. Effects of ranolazine on left ventricular regional diastolic function in patients with ischemic heart disease. Cardiovasc Drugs Ther. 1994; 8(5):741-747.
43. Moss A J, Zareba W, Schwarz K Q, Rosero S, McNitt S, Robinson J L. Ranolazine shortens repolarization in patients with sustained inward sodium current due to type-3 long-QT syndrome. J Cardiovasc Electrophysiol. 2008; 19(12): 1289-1293.
44. Scirica B M, Morrow D A, Hod H, Murphy S A, Belardinelli L, Hedgepeth C M, Molhoek P, Verheugt F W, Gersh B J, McCabe C H, Braunwald E. Effect of ranolazine, an antianginal agent with novel electrophysiological properties, on the incidence of arrhythmias in patients with non ST-segment elevation acute coronary syndrome: results from the Metabolic Efficiency With Ranolazine for Less Ischemia in Non ST-Elevation Acute Coronary Syndrome Thrombolysis in Myocardial Infarction 36 (MERLIN-TIMI 36) randomized controlled trial. Circulation. 2007; 116(15):1647-1652.
45. Clarke B, Wyatt K M, McCormack J G. Ranolazine increases active pyruvate dehydrogenase in perfused normoxic rat hearts: evidence for an indirect mechanism. J Mol Cell Cardiol. 1996; 28(2):341-350.
46. McCormack J G, Barr R L, Wolff A A, Lopaschuk G D. Ranolazine stimulates glucose oxidation in normoxic, ischemic, and reperfused ischemic rat hearts. Circulation. 1996; 93(1): 135-142.
47. Timmis A D, Chaitman B R, Crager M. Effects of ranolazine on exercise tolerance and Hb_{Alc} in patients with chronic angina and diabetes. Eur Heart J. 2006; 27(1):42-48.
The following represents a reference list numbered according to the citation numbering used in Example 7: 1. Owan et al., N Engl J. Med. 2006; 355:251-259.
2. Redfield et al., JAMA. 2003; 289:194-202.
3. Bursi et al., JAMA. 2006; 296:2209-2216.
4. Schocken et al., Circulation. 2008; 117:2544-2565.
5. Ouzounian et al., Nat Clin Pract Cardiovasc Med. 2008; 5:375-386.
6. Ahmed et al., Circulation. 2006; 114:397-403.
7. Hernandez et al., J Am Coll Cardiol. 2009; 53:184-192.
8. Massie et al., N Engl J. Med. 2008; 359:2456-2467.
9. Bers D M. Excitation-contraction coupling and cardiac contractile force. 2nd ed. Dordrecht; Boston: Kluwer Academic Publishers; 2001.
10. Ju et al., J Physiol. 1996; 497:337-347.
11. Undrovinas et al., Circ Res. 1992; 71:1231-1241.
12. Wu et al., Am J Physiol. 1994; 266:H1034-1046.
13. Ward et al., J Physiol. 1997; 500:631-642.
14. Hale et al., J Mol Cell Cardiol. 2008; 44:954-967.
15. Song et al., J Pharmacol Exp Ther. 2006; 318:214-222.
16. Flagg et al., Circ Res. 2009; 104:95-103.
17. Wolska et al., Am J Physiol. 1996; 270:H24-32.
18. Pohlmann et al., Circ Res. 2007; 101:928-938.
19. Davis et al., Circ Res. 2007; 100:1494-1502.
20. Silberman et al., Circulation. 2010; 121:519-528.
21. Cocco et al., J Cardiovasc Pharmacol. 1992; 20:131-138.
22. Pepine et al., Am J Cardiol. 1999; 84:46-50.
23. Kasner et al., Circulation. 2007; 116:637-647.
24. Maier et al., J Cardiovasc Pharmacol. 2009; 54:279-286.
25. de Tombe et al., Circ Res. 1995; 76:734-741.
26. Antzelevitch et al., Circulation. 2004; 110:904-910.
27. Kass, Cardiol Clin. 2000; 18:571-586.
28. Yellin and Meisner, Cardiol Clin. 2000; 18:411-433, vii.
29. Hiranandani et al., Am J Physiol Heart Circ Physiol. 2006; 291:H3130-3135.
30. Kuster et al., Free Radic Biol Med. 2010; 48:1182-1187.
31. Wagner et al., Cardiovasc Res. 2003; 60:404-412.
32. MacFarlane et al., Pflugers Arch. 1994; 428:561-568.
33. Wang et al., Am J Physiol Cell Physiol. 2008; 294:C1566-1575.
34. Monasky et al., Pflugers Arch. 2008; 456:267-276.
35. LeWinter et al., Heart Fail Rev. 2005; 10:249-257.
36. Sossalla et al., J Mol Cell Cardiol. 2008; 45:32-43.
37. Hwang et al., Circulation. 2009; 120:S16-21.
38. Gralinski et al., Cardiovasc Res. 1994; 28:1231-1237.
39. Maruyama et al., J Pharm Pharmacol. 2000; 52:709-715.
40. Undrovinas et al., J Cardiovasc Electrophysiol. 2006; 17:S169-S177.
41. Sabbah et al., J Card Fail. 2002; 8:416-422.
42. Rastogi et al., Am J Physiol Heart Circ Physiol. 2008; 295:H2149-2155.
43. Hayashida et al., Cardiovasc Drugs Ther. 1994; 8:741-747.
44. Moss et al., J Cardiovasc Electrophysiol. 2008; 19:1289-1293.
45. Hinken et al., Physiology (Bethesda). 2007; 22:73-80.
46. Tong et al., Circ Res. 2008; 103:974-982.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1-41. (canceled)

42. A method of treating diastolic dysfunction with preserved ejection fraction in a subject, wherein the subject does not suffer from a cardiac injury or structural heart disease, wherein the diastolic dysfunction with preserved ejection fraction is characterized by:
(i) a lack of increased late I_{Na} in cardiomyocytes,
(ii) an increase in myofilament calcium sensitivity, or
(iii) a combination thereof,
the method comprising administering to the subject a cardiac metabolic modifier in an amount effective to treat the diastolic dysfunction with preserved ejection fraction.

43. The method of claim 42, wherein the diastolic dysfunction with preserved ejection fraction is characterized by:
(i) a lack of change in calcium cycling or calcium handling in cardiomyocytes;
(ii) a lack of change in calcium concentration in resting myocytes;
(iii) a decrease in sarcomere length in resting myocytes;
(iv) an increase in diastolic tension; or
(v) a combination thereof.

44. A method of treating or preventing heart failure with preserved ejection fraction in a subject, wherein the subject does not suffer from a cardiac injury or structural heart disease, wherein the heart failure with preserved ejection fraction is characterized by:
(i) a lack of increased late I_{Na} in cardiomyocytes,
(ii) an increase in myofilament calcium sensitivity, or
(iii) a combination thereof,
the method comprising administering to the subject a cardiac metabolic modifier in an amount effect to treat or prevent heart failure with preserved ejection fraction.

45. The method of claim 44, wherein the heart failure with preserved ejection fraction is characterized by:
(i) a lack of change in calcium cycling or calcium handling in cardiomyocytes;
(ii) a lack of change in calcium concentration in resting myocytes;
(iii) a decrease in sarcomere length in resting myocytes;
(iv) an increase in diastolic tension; or
(v) a combination thereof.

46. The method of claim 42, wherein the cardiac injury or structural heart disease is selected from the group consisting of: ischemia, ischemia-reperfusion or artery occlusion-reperfusion, myocardial injury, myocardial toxicity, myocardial infarction, congenital heart lesion, valvular stenosis or valvular regurgitation, coronary artery disease, chronic angina, chronic stable angina, arrhythmias.

47. The method of claim 42, wherein the subject suffers from neither a New York Heart Association (NYHA) Class I heart failure nor a NYHA Class II heart failure.

48. The method of (claim 44), wherein the subject suffers from a NYHA Class III or IV heart failure.

49. The method of claim 42, wherein the subject does not present signs of myocardial wall thinning or regional wall motion abnormalities.

50-55. (canceled)

56. A method of treating acute decompensated heart failure in a subject in need thereof, comprising the step of administering to the subject a cardiac metabolic modifier in an amount effective to treat the acute decompensated heart failure in the subject.

57. The method of claim 42, wherein the cardiac metabolic modifier lowers myofilament calcium sensitivity.

58. The method of claim 57, wherein the cardiac metabolic modifier lowers calcium sensitivity of myofilaments of cardiac muscle.

59. The method of claim 42, wherein the cardiac metabolic modifier binds to myofilaments.

60. The method of claim 59, wherein the myofilaments are myofilaments of cardiac muscle.

61. The method of claim 42, wherein the cardiac metabolic modifier comprises a structure of Formula I: 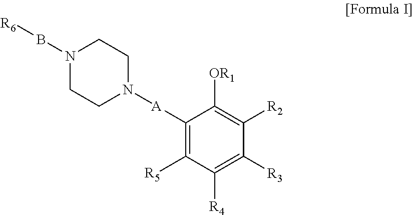
wherein A comprises a main chain of 1-8 atoms, each atom of which is independently C, O, N, or S, and each atom of which is optionally bound to an additional group selected from C1-C8 alkyl, C1-C8 alkoxy, OH, NH2, NH(C1-C4 alkyl) and SH;
wherein R₁ is H or a C1-C8 alkyl;
wherein each of R₂, R₃, R₄, and R₅ independently is H, a C1-C8 alkyl, or a C1-C8 alkoxy;
wherein B is H or comprises a main chain of 1-8 atoms, each atom of which is independently C, O, N, or S, and each atom of which is optionally bound to an additional group; and,
wherein R₆ is absent or phenyl, optionally substituted with 1 to 5 groups, each group of which is independently C1-C8 alkyl, C1-C8 alkoxy, or OH.

62. The method of claim 42, wherein A of Formula I is
(CH₂)₁₋₈
or comprises a structure of Formula IV: 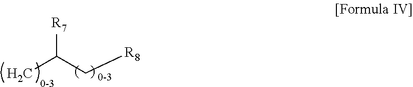
wherein R₇ is OH, C1-C4 alkyl, C1-C4 alkoxy, NH2, or NH(C1-C4 alkyl); and
wherein R₈ is O or NH.

63-66. (canceled)

67. The method of claim 42, wherein B comprises a structure of Formula V: 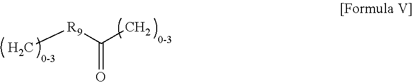
wherein R₉ is NH or O.

68-69. (canceled)

70. The method of claim 42, wherein the cardiac metabolic modifier comprises a compound of Formula II, or a pharmaceutically acceptable salt thereof or a conjugate thereof: 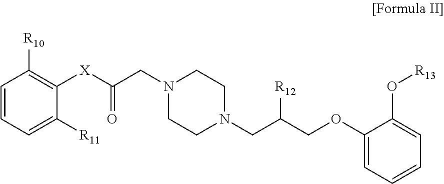
wherein each of R₁₀, R₁₁, and R₁₃ independently is C1-C3 alkyl,
wherein X is NH or O; and
wherein R₁₂ is OH or C1-C3 alkyl.

71. The method of claim 70, wherein the compound of Formula II is ranolazine, or a pharmaceutically acceptable salt thereof or a conjugate thereof.

72-73. (canceled)

74. The method of claim 42, wherein the cardiac metabolic modifier (i) inhibits fatty acid oxidation in cardiomyocytes, (ii) lowers myofilament calcium sensitivity, (iii) inhibits an ion channel, or (iv) a combination thereof.

75-86. (canceled)

87. The method of claim 42, wherein the cardiac metabolic modifier is intravenously administered to the subject, orally administered to the subject, or both.

88-93. (canceled)
